SEQUENCE LISTING (25 sequences)

SEQ ID NO: 1 - DNA, 27 bp, Artificial sequence
  Synthetic primer
tctagaactagtggatccccgcggatg

SEQ ID NO: 2 - DNA, 27 bp, Artificial sequence
  Synthetic primer
gcagactaggtcgacaagcttgatatc

SEQ ID NO: 3 - DNA, 23 bp, Artificial sequence
  Synthetic primer
aaggaataagggatgcctattca

SEQ ID NO: 4 - DNA, 23 bp, Artificial sequence
  Synthetic primer
ctactctggtgcggcgcgctggc

SEQ ID NO: 5 - DNA, 25 bp, Artificial sequence
  Synthetic primer
cgacgaggtgcagcgtgattaaggt

SEQ ID NO: 6 - DNA, 23 bp, Artificial sequence
  Synthetic primer
ctagctggcggcatcgaccatgc

SEQ ID NO: 7 - DNA, 28 bp, Artificial sequence
  Synthetic primer
gctctagaaaggaataagggatgcctat

SEQ ID NO: 8 - DNA, 30 bp, Artificial sequence
  Synthetic primer
cccaagcttctaacgctggcggccgagttc

SEQ ID NO: 9 - DNA, 30 bp, Artificial sequence
  Synthetic primer
cccaagcttctagcgcaggcgctggcgggc

SEQ ID NO: 10 - DNA, 30 bp, Artificial sequence
  Synthetic primer
cccaagcttctacaggcgctggcgggcgct

SEQ ID NO: 11 - DNA, 30 bp, Artificial sequence
  Synthetic primer
cccaagcttctagcgctggcgggcgctttc

SEQ ID NO: 12 - DNA, 38 bp, Artificial sequence
  Synthetic primer
gctctagaaaggaataagggatggtcagcctgatacgc

SEQ ID NO: 13 - DNA, 32 bp, Artificial sequence
  Synthetic primer
cccaagcttctactctggtgcggcgcgctggc

SEQ ID NO: 14 - DNA, 21 bp, Artificial sequence
  Synthetic primer
aacgccagcaattgcactgat

SEQ ID NO: 15 - DNA, 21 bp, Artificial sequence
  Synthetic primer
aatgtcagatgctcagctcgg

SEQ ID NO: 16 - DNA, 21 bp, Artificial sequence
  Synthetic primer
aagctacacaaaaagcctgga

SEQ ID NO: 17 - DNA, 21 bp, Artificial sequence
  Synthetic primer
aaaaagcctggatcactcatc

SEQ ID NO: 18 - DNA, 21 bp, Artificial sequence
  Synthetic primer
aacagggactcagggaccaga

SEQ ID NO: 19 - DNA, 21 bp, Artificial sequence
  Synthetic primer
aaatgaatgttcctggggcgc

SEQ ID NO: 20 - DNA, 21 bp, Artificial sequence
  Synthetic primer
aatctgaaggtgatgacttca

SEQ ID NO: 21 - DNA, 21 bp, Artificial sequence
  Synthetic primer
aacaccttgactaaaccgact

SEQ ID NO: 22 - DNA, 21 bp, Artificial sequence
  Synthetic primer
aagagaaatcgaaaccgaaaa

SEQ ID NO: 23 - DNA, 21 bp, Artificial sequence
  Synthetic primer
aagaacccaattaagcgcaag

SEQ ID NO: 24 - protein, 4 aa, Artificial sequence
  Synthetic peptide
YPTF

SEQ ID NO: 25 - protein, 5 aa, Artificial sequence
  Synthetic peptide
YYPFF

(19) United States
(12) Patent Application Publication
(10) Pub. No.: US 2008/0194611 A1
(43) Pub. Date: Aug. 14, 2008

(54) Modulation of Cell Barrier Dysfunction

(76) Inventors: John C. Alverdy, Glenview, IL (US); Jonathan Moss, Chicago, IL (US); Mark W. Lingen, Oak Park, IL (US); Patrick A. Singleton, Chicago, IL (US); Joe G.N. Garcia, Chicago, IL (US)

Correspondence Address: MICHAEL BEST & FRIEDRICH LLP, ONE SOUTH PINCKNEY STREET, P O BOX 1806, MADISON, WI, 53701, US

(21) Appl. No.: 11/914,984

(22) PCT Filed: May 6, 2006
(86) PCT No.: PCT/US2006/021604

§ 371 Date: Feb. 14, 2008

Related U.S. Application Data
(60) Provisional application No. 60/687,568, filed on Jun. 3, 2005.
(60) Provisional application No. 60/731,009, filed on Oct. 28, 2005.
(60) Provisional application No. 60/760,851, filed on Jan. 20, 2006.

(30) Foreign Application Priority Data
Mar. 7, 2006 (US) PCTUS2006007892

(51) Int. Cl. A61K 31/485; A61K 31/445; A61P 31/00; A61P 17/00

(52) U.S. Cl. 514/282; 514/331

(57) ABSTRACT

The invention provides prophylactic and therapeutic methods for administering a μ-opioid receptor antagonist to treat cell barrier diseases and disorders, such as endothelial and epithelial cell barrier diseases and disorders. The diseases or disorders may amenable to the methods include inflammation, such as acute lung injury, atherosclerosis, gut-derived sepsis, a burn injury, neonatal necrotizing enterocolitis, severe neutropenia, toxic colitis, inflammatory bowel disease, enteropathy, transplant rejection, pouchitis, pig belly (pig-bel), Pseudomonas-mediated opthalmologic infection, Pseudomonas-mediated otologic infection and Pseudomonas-mediated cutaneous infection. More generally, epithelial cell barrier disorders are contemplated as amenable to the prophylactic and therapeutic methods. The diseases and disorders may be induced by microbial pathogens, including bacterial pathogens such as Pseudomonas aeruginosa. The invention further provides prophylactic and therapeutic methods for inhibiting the expression of bacterial PA-I lectin/adhesin and for inhibiting bacterial MvfR activity levels. Also provided is a method of using a μ-opioid receptor antagonist in the manufacture of a medicament for use in the methods described herein.

### CROSS REFERENCE TO RELATED APPLICATION

This application claims the benefit of Provisional U.S. Patent Application Nos. 60/687,568, filed Jun. 3, 2005, 60/731,009, filed Oct. 28, 2005, and 60/760,851, filed Jan. 20, 2006. The application also claims the priority benefit of international (PCT) patent application PCT/US06/07892, which designates the United States. Each of the above-identified patent applications is hereby expressly incorporated herein by reference in its entirety.

The invention was made with U.S. Government support under contract nos. DE12322, DE00470, R01-GM-62344-01 and DE015830 awarded by the National Institutes of Health. The U.S. Government has certain rights to this invention.

### FIELD OF THE INVENTION

The invention generally relates to the field of prophylactic and therapeutic use of opioid receptor antagonists in modulating cell barrier dysfunction characteristic of a disorder or disease afflicting vertebrates (e.g., mammals) such as humans.

### BACKGROUND

Vertebrate (e.g., mammalian) cell barrier dysfunction results in a change in permeability of a cell barrier contributing to the internal compartmentalization of a multicellular organism and/or to the segregation of internal and external environments of such an organism. Typically, cell barrier dysfunctions are revealed as an increase in the permeability of a particular cell layer, such as the layer of endothelial cells found in the vasculature of higher eukaryotes or the layer of epithelial cells found in tissues exposed to the external environment, including the skin, lung and gut. A variety of disorders and diseases afflicting vertebrates such as humans can involve cell barrier dysfunction. Collectively, these maladies affect the quality of life of humans and other animals (e.g., domesticated animal, zoo or exotic animals, pets) while contributing to the increasingly burdensome cost of health care. In the following description, a particular cell type, such as endothelial cells or epithelial cells, will be used for ease of exposition, with the understanding that cell barrier dysfunction applies to a variety of cell types, including the aforementioned endothelial and epithelial cells.

For example, endothelial cells provide a semi-selective barrier between the blood and underlying vasculature. Disruption of this barrier results in increased vascular permeability and organ dysfunction. For example, the inflammatory process increases macromolecular transport by decreasing cell-cell and cell-matrix adhesion and by increasing centripetally directed tension, resulting in the formation of intercellular gaps. Agents that enhance endothelial cell barrier function provide a desirable therapeutic strategy for a variety of inflammatory diseases, atherosclerosis and acute lung injury.

Cell barrier dysfunction can be caused or exacerbated by a variety of factors, including microbial pathogens, and by a variety of agents, including thrombin, ionomycin, LPS, and the like. Microbial pathogens such as P. aeruginosa can express various peptides and virulence factors that can disrupt barrier function. Microbiologists have long recognized that many bacteria activate their virulence genes in response to ambient environmental cues. In general such physico-chemical cues signal environmental stress or adversity, such as changes in redox status, pH, osmolality, and the like. For example, P. aeruginosa and other bacteria can express a lectin/adhesin PA-I. The distribution of PA-I in bacteria can be either primarily intracytoplasmic or extracellular, depending on its environment. When bacteria are grown in ideal growth conditions, about 85% of PA-I is located intracellularly with small, but significant, amounts located within the cytoplasmic membrane, on the outer membrane, and in the periplasmic space. In sharp contrast, within the intestinal tract of a stressed host, PA-I abundance is increased and localizes to the outer membrane, facilitating the adherence of P. aeruginosa to the intestinal epithelium. In addition, there is evidence that free PA-I is shed into the extracellular milieu and can be detected at concentrations as high as 25 μg/ml in both culture supernatants and sputum from P. aeruginosa infected lungs. This finding is of considerable importance, as treatment of cultured epithelial cells (e.g. T-84, Caco-2bbe, MDCK, airway epithelial cells) with 25 μg/ml purified PA-I causes a profound permeability defect. This effect is also seen in the intestinal tract in vivo. These effects are of clinical significance because P. aeruginosa is the most conmon gram-negative bacterium isolated among cases of nosocomial infection and carries the highest reported fatality rate of all hospital acquired infections. The mere presence of this pathogen within the intestinal tract of a critically ill patient is associated with a four-fold increase in mortality, independent of its dissemination to remote organs.

Although there has been very little work on specific membrane sensors that activate virulence gene expression in P. aeruginosa, two sensor proteins located within the cell membrane of P. aeruginosa, termed CyaB, GacS have been shown to respond to three known external signals, host cell contact, low calcium, and beet seed extract. CyaB (via cAMP) and GacS⁴ (via phosphorylation), activate the transcriptional regulators Vfr and GacA respectively, which, along with the cell density sensitive PcrA, exert global regulatory influences on two central systems for virulence gene regulation in P. aeruginosa, the QS and RpoS signaling systems. Mutant strains defective in CyaB and GacS have attenuated lethality in mice following lung instillation.

Host cellular elements such as seed extract and cell contact, activate the membrane biosensors CyaB and GacS. These two component transmembrane alarm systems then activate two main global regulators of virulence, Vfr and GacA. Vfr is involved in the activation of LasRI which in turn promotes the activation of the RhlRI system of QS. GacA induces the transcription of lasR and rhlR genes, and is also implicated in the expression of rpoS. Finally a third system, PQS, induces expression of both RhlR and RpoS. Thus, activation of any of the membrane biosensors could lead to the expression of PA-I with the involvement of a number of different pathways.

Opioids comprise a large group of compounds that are distributed in virtually every tissue of the body and are abundantly released in response to various stress conditions; for example dynorphin and β-endorphin appear to be the predominantly released endogenous opioids following stress (S. Yoshida, et al., Surg Endosc 14, 137 (2000), C. Sternini, S. Patierno, I. S. Selmer and A. Kirchgessner, Neurogastroenterol Motil 16 Suppl 2, 3 (2004)). Morphine and morphine derivatives (opiates) as well as morphine-like compounds (opioids) are among the most widely used analgesic drugs in the world and are often administered at high doses even at continuous dosing intervals in post-operative care, chronic pain management, and in critically ill patients such as patients with advanced cancer or AIDS. Intravenously applied morphine has been demonstrated to accumulate at tissues sites of bacterial infection such as the intestinal mucosa, at concentrations as high as 100 μM (P. Dechelotte, A. Sabouraud, P. Sandouk, I. Hackbarth and M. Schwenk, Drug Metab Dispos 21, 13 (1993)) and has been shown to readily cross the intestinal wall into the lumen (M. M. Doherty and K. S. Pang, Pharm Res 17, 291 (2000)). Therefore it is likely that an opportunistic pathogen such as P. aeruginosa, which is present in greater than 50% of the intestines of critically ill patients within 3 days of hospitalization, is exposed to both endogenously released and exogenously applied opioid compounds. Clinical data suggest that bacterial transmigration across the gut may lead to increased rates of sepsis in burn or ICU patients who have diminished gut motility.

The association of opioids and infection is well established (Risdahl, et al., J Neuroimmunol 83:4 (1998)), including evidence that opioids enhance HIV infection of human macrophages by upregulating CCR5 receptor. Ho et al., J. Pharm. And Exp. Ther. 307:1158-1162 (2003). Nonetheless, most of the work in this area has focused on the suppressive effects of opioids on the immune system (Eisenstein, et al., Adv Exp Med Biol 493, 169 (2001)). Although opioids have been shown to suppress a variety of immune cells resulting in impaired clearance of bacteria and enhanced mortality in animals (Wang, et al., J Leukoc Biol 71, 782 (2002)), it has not been previously considered that opioid compounds might also directly activate the virulence of bacteria.

Opioids and opioid antagonists such as morphine and DAMGO (([D-Ala², N-MePhe⁴, Gly⁵-ol], a mu opioid enkephalin) bind to the mOP-R present in the central nervous system (CNS) and peripheral tissue. The mOP-R is expressed in a variety of cell types including endothelial cells and epithelial cells. The mOP-R is a G protein-coupled receptor with multiple isoforms resulting from alternative splicing of mRNA encoded from a single gene. Most mu opioid receptor antagonists, including naloxone, exist in an uncharged state and readily pass through the blood-brain barrier (BBB) to reverse CNS-dependent analgesic effects. MNTX, however, is a charged molecule that is known to be unable to penetrate the BBB. The effects of MNTX and other quaternary derivatives of noroxymorphone (QDNM) on cell barrier regulation have not been reported.

Several receptors have been implicated in cell (e.g., endothelial cell) barrier function. One important receptor family is the sphingosine-1-phosphate (SIP) receptors (also called Edg receptors, endothelial differentiation gene). S1P binds to the plasma membrane G protein-coupled S1P receptors 1 (Edg1), 2 (Edg5), 3(Edg3), 4 (Edg6) and 5 (Edg8) expressed in a variety of cell types including endothelium. Human endothelial cells exhibit high expression of S1P1 and S1P3 with S1P1 signaling coupled to the Gi pathway and Rac1 activation, whereas S1P3 signaling couples to the Gi, Gq/11 and G12/13 pathways and activates RhoA to a much greater extent than Rac1. S1P1 receptor-dependent activation of Rac1 has been shown to promote vascular integrity. In contrast, S1P3 receptor-dependent activation of RhoA can potentially regulate endothelial cell barrier disruption.

Src (pp60Src, c-Src tyrosine kinase) is a non-receptor tyrosine kinase that contains an amino-terminal myristoylation site, Src Homology (SH) sites (i.e., SH2 and SH3), a tyrosine kinase catalytic domain and regulatory tyrosine phosphorylation sites. Activation of Src promotes endothelial cell barrier disruption and endothelial cell contraction. Inhibition of Src attenuates edema and tissue injury after myocardial infarction.

Protein tyrosine phosphatases (PTPs) are a diverse superfamily encoded by over 100 genes that regulate a myriad of cellular events. One PTP highly expressed in lung endothelium is the receptor-like protein tyrosine phosphatase mu (RPTPμ). Structurally, RPTPμ is composed of extracellular MAM (Meprin-A5-protein-M-type-RPTP (RPTPμ), Immunoglobulin (Ig)-like and Fibronectin type 3 (FN3)-like domains and intracellular PTP catalytic domains. RPTPμ is localized at endothelial cell junctions and regulates vascular integrity.

While in vitro assays have been enormously useful and continue to provide important information on the mechanisms of bacterial pathogenesis, they cannot accurately reproduce all aspects of the host pathogen interaction, as a pathogen will encounter several radically different environments in the host at various points during infection. Consequently, a gene that seems important in in vitro studies, may not be important in vivo, and genes that appear unimportant in an in vitro assay may play a critical role during a natural infection. Furthermore, it has recently been shown that bacteria growing on the surface of solid agar have a markedly different physiology from those in broth, as judged by differential regulation of nearly one-third of their functional genome. Therefore, experiments must now be designed that control for the variables of the growth environment and host environment, while at the same time allowing for measurements of gene expression patterns and phenotype analysis which are not possible in more traditional models, such as stressed mice.

Severe sepsis continues to be the number one cause of mortality among critically ill patients. Interventions to attenuate regulatory arms of the systemic immune response have resulted in clinical failure. Alternatively, newer and more powerful antibiotics have resulted in the emergence of highly resistant stains of bacteria for which there is no foreseeable therapy other than de-escalating their use. P. aeruginosa is now on the international list of emerging resistant pathogens posing a real and present danger to the public.

Thus, a need continues to exist in the art for methods of preventing, mitigating or treating cell barrier dysfunction, including endothelial cell barrier dysfunction and epithelial cell barrier dysfunction. Further, the need for compositions and methods to alleviate a symptom associated with a cell barrier dysfunction condition has not been satisfied.

### SUMMARY

The invention satisfies at least one of the foregoing needs in the art in providing compositions and methods for preventing or treating cell barrier dysfunction by administering an effective amount of an opioid receptor antagonist (OP-RA). The invention is directed in important embodiments to preventing or treating an endothelial or epithelial cell barrier dysfunction. Specifically, the invention relates to the cell barrier dysfunction inhibitory effect of opioid receptor antagonists, including peripherally restricted antagonists (e.g., polar or charged antagonists typified by methylnaltrexone) as well as centrally acting antagonists. The methods are effective in preventing or treating the barrier dysfunction and attendant conditions and symptoms arising therefrom, associated with a variety of diseases and disorders, such as inflammation, atherosclerosis, and microbial pathogenesis. As particular nonlimiting examples, the conditions with which the cell barrier dysfunction occurs may be gut-derived sepsis, a burn injury, a chemical contact injury, acute lung injury, neonatal necrotizing enterocolitis, severe neutropenia, toxic colitis, inflammatory bowel disease, Crohn's disease, enteropathy, transplant rejection, pouchitis, pig-bel, uremic pericardial effusion, leakage in the vitreous of the eye, macular degeneration, retinal dysfunction, and infection (e.g., viral infection, bacterial infection, opportunistic bacterial infection, Clostridium dificile infection, Pseudomonas aeruginosa infection, Pseudomonas-mediated opthalmologic infection, Pseudomonas-mediated otologic infection and Pseudomonas-mediated cutaneous infection).

The opioid receptor antagonists useful in the inventions described herein are set forth more comprehensively in the detailed description below, which description is incorporated into this summary by reference. Examples of suitable opioid receptor antagonists include heterocyclic amine compounds that belong to several classes of compounds. One class is the tertiary derivatives of morphinan and, in particular, the tertiary derivatives of noroxymorphone. In one embodiment, the tertiary derivative of noroxymorphone, e.g., naloxone or naltrexone, is contemplated. Another class is the quaternary derivatives of morphinan and, in particular, the quaternary derivatives of noroxymorphone. Another class is the N-substituted piperidines. Another class is the quaternary derivatives of benzomorphans. In particular embodiments, the opioid receptor antagonist is a peripheral μ-opioid receptor antagonist, such as N-methylnaltrexone, alvimopan, ADL 08-0011, a piperidine-N-alkylcarboxylate, a quaternary morphinan, an opium alkaloid derivative or a quaternary benzomorphan compound. Further, the quaternary morphinan compound may be a quaternary salt of N-methylnaltrexone, N-methylnaloxone, N-methylnalorphine, N-diallylnormorphine, N-allyllevallorphan or N-methylnalmefene. In some embodiments, the quaternary benzomorphan compound is 2′-hydroxy-5,9-dimethyl-2,2-diallyl-6,7-benzomorphanium-bromide; 2′-hydroxy-5,9-dimethyl-2-n-propyl-6,7-benzomorphan; 2′-hydroxy-5,9-dimethyl-2-allyl-6,7-benzomorphan; 2′-hydroxy-5,9-dimethyl-2-n-propyl-2-allyl-6,7-benzomorphanium bromide; 2′-hydroxy-5,9-dimethyl-2-n-propyl-2-propargyl-6,7-benzomorphanium bromide; or 2′-acetoxy-5,9-dimethyl-2-n-propyl-2-allyl-6,7-benzomorphanium bromide. In some embodiments, the method further comprises administration of a high molecular weight polyethylene glycol-like compound having an average molecular weight of at least 15 kilodaltons.

In preferred embodiments, the antagonist is a mu opioid receptor antagonist. In some embodiments, the antagonist is a peripheral opioid receptor antagonist, e.g., MNTX, which may also inhibit VEGF, platelet-derived growth factor (PDGF), sphingosine-1-phosphate (S1P) and/or hepatocyte growth factor (HGF)-stimulated or induced cell barrier dysfunction.

As mentioned, in some embodiments of the invention, the opioid receptor antagonist is a mu opioid receptor antagonist. In other embodiments, the opioid receptor antagonist is a kappa opioid receptor antagonist. The invention also encompasses administration of more than one opioid receptor antagonist, including combinations of mu opioid receptor antagonists, combinations of kappa opioid receptor antagonists and combinations of mu and kappa opioid receptor antagonists, for example, a combination of methylnaltrexone and alvimopan (or ADL 08-0011), or a combination of naltrexone and methylnaltrexone.

The invention described herein involves the prevention and/or treatment of cell barrier dysfunction in vertebrates, and more preferably mammals. Important subjects or “patients” to be treated are farm animals (e.g., horses, goats, cows, sheep, pigs, fish and chickens), domestic animals (dogs and cats) and humans.

The invention described herein involves prevention or treatment of cell barrier dysfunction. Prevention as used herein means administration of an opioid receptor antagonist to a patient at risk of a cell barrier dysfunction in an amount effective to inhibit the appearance of, to lessen the development of or to prevent altogether the appearance of a symptom or adverse medical condition arising from the cell barrier dysfunction. Treatment as used herein means administration of an opioid receptor antagonist to a patient having or believed to have a condition or symptom associated with a cell barrier dysfunction in an amount effective to inhibit, to halt the further development of, to lessen or to eliminate altogether a symptom or adverse medical condition arising from the cell barrier dysfunction.

An opioid receptor antagonist, such as a mu opioid receptor antagonist (mOP-RA) like methylnaltrexone (MNTX), inhibits cell barrier dysfunction. For example, mu opioid receptor antagonists, including MNTX, inhibit opiate-, thrombin- and LPS-induced endothelial cell barrier dysfunction by mu opioid receptor (mOP-R)-dependent, and -independent, mechanisms. The mOP-R-independent mechanisms of mOP-RA (e.g., MNTX)-induced endothelial cell barrier regulation include activation of receptor-like protein tyrosine phosphatase mu (RPTPμ) and inhibition of thrombin- and LPS-induced, Src-dependent, S1P3 receptor transactivation (tyrosine phosphorylation). Thus, mOP-RAs such as MNTX are useful as cell barrier protective agents.

The invention described herein provides methods for enhancing cell barrier function (e.g., endothelial and/or epithelial cell barrier function), comprising administering to a patient in need of such treatment a composition comprising an effective amount of one or more opioid receptor antagonists. For example, cell barrier function can be disrupted in certain inflammatory syndromes. Thus, the invention provides a method of preventing or treating inflammatory syndromes, e.g., acute lung injury, as well as atherosclerosis and microbial pathogenesis (e.g., infection), which are characterized by a cell barrier dysfunction, typically an epithelial or endothelial cell barrier dysfunction. The methods described herein also involve treating or preventing a symptom arising from cell barrier dysfunction associated with any of these diseases.

In connection with all aspects of the inventions described herein, the patient preferably is a human. In some embodiments, the human patient is free of cancer, and/or is not in a methadone maintenance program, and/or is not immunosuppressed. In some embodiments, the patient is not experiencing post operative bowel dysfunction. The patient may be, or may not be, on concurrent opioid therapy, depending on the particular disorder the patient has, the severity of the disorder, and the need the patient has for pain management. In some embodiments, the patient is taking concurrent opioid therapy. In some embodiments, the patient is not taking concurrent opioid therapy. In some embodiments, the patient is taking concurrent chronic opioid therapy. In some embodiments, the patient is not taking concurrent chronic opioid therapy. In some embodiments, the patient is receiving a dose of an opioid antagonist that is independent of any dose of opioid therapy concurrently administered.

In some embodiments, the effective amount is such that the patient has effective circulating blood plasma levels of the opioid antagonist continuously for at least 1 week, at least 2 weeks, at least three weeks and, even at least 4 weeks. In one embodiment, the opioid antagonists are used peri-operatively. By peri-operatively, it is meant before (e.g., in preparation for), during, and/or immediately after a surgical procedure (i.e., up to three or even up to five days). The opioid antagonists act to attenuate, preserve, or maintain the cell barrier function, thereby inhibiting inflammation, inhibiting infection including opportunistic infection, and inhibiting recurrence of and/or the metastasis of a tumor in the case of a surgical procedure involving removal of a tumor- and particularly a tumor that is not an endothelial cell tumor.

The invention also includes the co-administration of the opioid antagonists with agents that are not opioid antagonists, but which are nonetheless useful in treating a disorder, condition or symptom associated with a cell barrier dysfunction. Examples of such agents include anti-cancer agents, anti-neovascularization agents (for example, anti-VEGF monoclonal antibody), anti-infective agents (e.g., antibacterial agents and anti-viral agents), anti-inflammatory agents, anti-atherosclerotic agents, anti-thrombotic agents, and the like.

An aspect of the invention provides a method of treating a disorder characterized by a cell barrier dysfunction comprising administering to a subject free of an opioid-induced side effect an effective amount of a μ-opioid receptor antagonist. The opioid-induced side effects include opioid-induced constipation, irritable bowel syndrome, post-operative ileus or bowel dysfunction, opioid-induced nausea, opioid-induced vomiting, urinary retention, delayed gastrointestinal tract emptying, reduced gastrointestinal tract motility and opioid-induced suppression of the immune system. In some embodiments, the cell barrier dysfunction may be attributable to endothelial cells, epithelial cells, or both types of cells.

Another aspect of the invention provides a method of reducing the risk of developing a disorder characterized by a cell barrier dysfunction comprising administering to a subject at risk of developing the disorder a prophylactically effective amount of an opioid receptor antagonist.

Another aspect of the invention provides a method of reducing a symptom associated with a cell barrier disorder, comprising administering to a subject in need thereof an opioid receptor antagonist, wherein the compound is administered in an amount effective to reduce at least one symptom of the disorder.

Another aspect of the invention is a method of preventing tumor cell metastasis comprising peri-operatively administering an effective amount of an opioid receptor antagonist to a patient having a tumor amenable to surgical intervention. In some embodiments the tumor cell is not an endothelial cell tumor.

Another aspect of the invention provides a method for preventing an infection or for lowering the risk of an infection by administering to a patient in need of such treatment an effective amount of an opioid receptor antagonist. In some embodiments, the patient has a traumatic injury, such as an internal injury, a surgery, an acute lung injury, or a burn. In other embodiments, the patient is subjected to high levels of stress. In some embodiments the infection is from an opportunistic infectious agent. In some embodiments the infection is a bacterial infection. In some embodiments the infectious agent is Clostridium dificile, or another bacterium capable of developing a virulent phenotype, such as Pseudomonas aeruginosa.

Another aspect of the invention provides a method of inhibiting the expression of a bacterial PA-I lectin/adhesin by a bacterium in a patient comprising administering an effective amount of an opioid receptor antagonist to a subject at risk of developing or suffering from bacterial pathogenesis. Any known bacterial pathogen, such as Clostridium dificile, or bacterium capable of developing a virulent phenotype, such as Pseudomonas aeruginosa, that is further capable of expressing a PA-I lectin/adhesin ortholog is contemplated.

Another aspect of the invention provides a method for modulating the activity of a bacterial MvfR protein comprising administering an effective amount of an opioid receptor antagonist to a subject at risk of developing or suffering from bacterial pathogenesis.

Another aspect of the invention provides a method of decreasing the permeability of, or preventing the increase in permeability of, an epithelium to a bacterial toxin comprising administering to a subject an amount of an opioid receptor antagonist effective in reducing, or inhibiting an increase in, transepithelial cell electrical resistance.

Another aspect of the invention provides a method for preventing or treating sepsis by administering to a patient in need of such treatment an effective amount of an opioid receptor antagonist.

Another aspect of the invention provides a method for preventing or treating inflammation by administering to a patient in need of such treatment an effective amount of an opioid receptor antagonist. In some embodiments, the patient has inflammation from a traumatic injury, such as an internal injury, a surgery, an acute lung injury, or a burn. In other embodiments, the patient has inflammation from an infection. In some embodiments the infection is a bacterial infection. In some embodiments the infectious agent is Clostridium dificile, or another bacterium capable of developing a virulent phenotype, such as Pseudomonas aeruginosa.

Another aspect of the invention provides a method of mitigating a cell barrier dysfunction free of μ-opioid receptor-dependent effects, comprising administering to a subject free of an opioid-induced side effect an effective amount of a peripheral μ-opioid receptor antagonist. In some embodiments, the peripheral μ-opioid receptor antagonist is N-methylnaltrexone. Also in some embodiments, the cell barrier dysfunction is induced by an inducing agent selected from the group consisting of thrombin and bacterial lipopolysaccharide. This aspect of the invention also extends to methods wherein a protein phosphatase is activated in the cell, such as methods in which an S1P3 receptor phosphorylation is reduced. In some embodiments of this method, a protein tyrosine phosphatase, such as a receptor protein tyrosine phosphatase μ, is activated.

Yet another aspect of the invention is a method of mitigating a cell barrier dysfunction induced by transactivation of a S1P3 receptor, comprising administering to a subject free of an opioid-induced side effect an effective amount of a peripheral μ-opioid receptor antagonist. In some embodiments, the peripheral μ-opioid receptor antagonist is N-methylnaltrexone.

Still another aspect according to the invention is a method of using an opioid receptor antagonist in the preparation of a medicament for treating, ameliorating, or preventing a disorder or a symptom of a disorder selected from the group consisting of inflammation, atherosclerosis, acute lung injury, gut-derived sepsis, a burn injury, a chemical contact injury, neonatal necrotizing enterocolitis, severe neutropenia, toxic colitis, inflammatory bowel disease, Crohn's disease, enteropathy, transplant rejection, pouchitis, pig-bel, uremic pericardial effusion, leakage in the vitreous of the eye, macular degeneration, retinal dysfunction, infection (e.g., viral infection, bacterial infection, opportunistic bacterial infection, Clostridium dificile infection, Pseudomonas aeruginosa infection, Pseudomonas-mediated opthalmologic infection, Pseudomonas-mediated otologic infection and Pseudomonas-mediated cutaneous infection).

Using a combination of in vivo and molecular methods, surgical stress has been shown to cause the release of host cell-derived Bacterial Signaling Compounds (host stress-derived BSCs) into the intestinal lumen that directly activate the virulence machinery of P. aeruginosa. The release of such host-derived BSCs, which include morphine, K and 6 opioid receptor agonists, and Interferon gamma (IFN-γ), can shift the phenotype of P. aeruginosa, or other members of the normal intestinal flora, from that of indolent colonizer to lethal pathogen. Exposure of P. aeruginosa to host stress-derived BSCs induces the expression of the PA-I lectin/adhesin (PA-I), a key virulence gene involved in lethal gut-derived sepsis in mice. In at least some instances, induction of PA-I expression is mediated by a transcriptional regulator of virulence gene expression, MvfR. PA-I induces an epithelial permeability defect to at least two potent cytotoxins of this organism, exotoxin A and elastase, causing lethal gut-derived sepsis and other disorders characterized by an epithelial cell barrier dysfunction. The data provide evidence for a model in which opportunistic pathogens sense host stress and vulnerability by perceiving key mediators released by the host into the intestinal tract during stress, such as the stress resulting from surgery. These host stress-derived compounds directly activate critical genes in P. aeruginosa leading to enhanced virulence.

Opioids, released in increased amount during physiological stress, directly induce the expression of several quorum sensing-dependent virulence factors in P. aeruginosa, such as pyocyanin, biofilm, and the lectin/adhesin PA-I. Specifically, U-50,488 (bremazocine, i.e., trans-3,4-dichloro-N-methyl-N[2-(1-pyrrolidinyl)cyclohexyl]benzeneacetamide methanesulfonate, an exemplary κ-opioid receptor agonist, induces pyocyanin production in P. aeruginosa via the global virulence transcriptional regulator MvfR. U-50,488 also induces pyocyanin at cell densities below those that would normally produce pyocyanin. These findings indicate that opioids, whether exogenous or endogenous, function as host stress-derived bacterial signaling molecules capable of activating a virulence response in P. aeruginosa. One aspect according to the invention provides a method of treating a disorder characterized by a barrier dysfunction (e.g., an epithelial cell or an endothelial cell) comprising administering, to a subject receiving at least one opiate or experiencing release of at least one endogenous opioid (e.g., an endorphin) but not experiencing an opioid-induced side effect, an effective amount of a μ-opioid receptor antagonist. An opioid-induced side effect includes an opioid-induced constipation, irritable bowel syndrome, post-operative ileus or bowel dysfunction, opioid-induced nausea, opioid-induced vomiting, urinary retention, delayed gastrointestinal tract emptying, reduced gastrointestinal tract motility and opioid-induced suppression of the immune system. In some embodiments, the patient will not be undergoing treatment for cancer or methadone treatment for drug addiction. In some embodiments, the subject will not be receiving or experiencing an exogenous or an endogenous opioid.

In an aspect, the invention thus provides a method of reducing the risk of developing a disorder characterized by a cell barrier dysfunction (e.g., an epithelial cell or an endothelial cell) comprising administering to a subject at risk of developing the disorder a prophylactically effective amount of a μ-opioid receptor antagonist. Another aspect of the invention is drawn to a method of reducing a symptom associated with a cell barrier disorder (e.g., an epithelial or endothelial cell barrier disorder), comprising administering to a subject in need thereof a μ-opioid receptor antagonist, wherein the compound is administered in an amount effective to reduce at least one symptom of the disorder. Another aspect of the invention is a method of inhibiting the expression of a bacterial PA-I lectin/adhesin comprising administering an effective amount of a p-opioid receptor antagonist to a subject at risk of developing or suffering from bacterial pathogenesis. In some embodiments of this method, the bacterial PA-I lectin/adhesin is found in a bacterium residing in a mammalian intestine. In some embodiments of this aspect, the bacterial PA-I lectin/adhesin is a Pseudomonad PA-I lectin/adhesin. An important Pseudomonad is Pseudomonas aeruginosa. Another aspect of the invention is directed to a method of modulating the activity of a bacterial MvfR protein comprising administering an effective amount of a μ-opioid receptor antagonist to a subject at risk of developing or suffering from bacterial pathogenesis. In some embodiments, the bacterial MvfR protein is found in a bacterium residing in a mammalian intestine. Also in some embodiments, the bacterial MvfR protein is a Pseudomonad MvfR protein, preferably a Pseudomonas aeruginosa MvfR protein. In another enumerated aspect, the invention provides a method of decreasing the permeability of, or preventing the increase in permeability of, an epithelium to a bacterial toxin comprising administering to a subject an amount of a μ-opioid receptor antagonist effective in reducing, or inhibiting an increase in, transepithelial cell electrical resistance (i.e., transcellular electrical resistance of an epithelium). An epithelium in the context of this aspect of the invention comprises at least two epithelial cells. In some embodiments, the epithelial cells are intestinal epithelial cells. Also contemplated in this aspect of the invention is a subject that comprises a microbial pathogen, such as Pseudomonas aeruginosa or Clostridium dificile.

In all of the aspects of the invention, any mode of administering the opioid receptor antagonist that is known in the art is contemplated, and in particular, delivery by parenteral, oral, subcutaneous, transcutaneous, subcutaneous implantation, intramuscular, intravenous, intrathecal, intraocular, intravitreous, opthalmologic, intraspinal, intrasynovial, topical, rectal, transepithelial including transdermal, buccal, sublingual, intramuscular, intracavity, and aural routes, as well as by nasal inhalation including via insufflation and aerosol. Microbial pathogens, such as P. aeruginosa, not only inhabit the intestinal tract, these pathogens are also capable of opthalmologic, otologic and cutaneous infection of subjects (e.g., humans). Thus, the invention comprehends administering the opioid receptor antagonist by direct routes, e.g., as by topical delivery, cutaneous delivery, intravitreous delivery, and intracerebroventricular delivery, to achieve localized, therapeutically useful concentrations of the antagonist. In addition, the invention comprehends treatment of any disorder caused, at least in part, by a microbial pathogen such as P. aeruginosa, which includes Pseudomonas-mediated opthalmologic, Pseudomonas-mediated otologic or Pseudomonas-mediated cutaneous disorders, by administering an opioid receptor antagonist through conventional systemic routes, including intravitreously, intracerebroventricularly, and topically (e.g., opthalmologically, ontologically, cutaneously), at levels sufficient to achieve therapeutically useful systemic levels of the antagonist.

Other features and advantages of the present invention will be better understood by reference to the following detailed description, including the drawing and the examples.

### BRIEF DESCRIPTION OF THE DRAWING

FIG. 1 is a panel of graphs, bar graphs and immunoblots showing that IFN-γ induces the expression of the PA-I lectin in P. aeruginosa.

FIG. 2 is a panel of bar graphs showing that the presence of rhlI and rhlR, core quorum sensing signaling elements in P. aeruginosa, are required for the PA-I expression and pyocyanin production in response to IFN-γ.

FIG. 3 is a panel of graphs, an epimicrograph, immunoblots and MS/MS spectra showing the identification of the IFN-γ binding site to solubilized membrane fractions of P. aeruginosa (PAO1).

FIG. 4 is a panel of bar charts and graphs showing the binding characteristics of the IFN-γ to membrane fractions of P. aeruginosa (PAO1).

FIG. 5 is a panel of graphs, bar charts and immunoblots showing that IFN-γ binds to OprF and induces PA-1 expression.

FIG. 6 is a panel of bar graphs and graphs showing that MvfR plays a key role in the effect of U-50,488 and C4-HSL on PCN production.

FIG. 7 is a bar graph showing the inhibition of morphine-induced PA-I lectin/adhesin expression in the separate presences of μ-opioid receptor antagonists methylnaltrexone (MNTX) and naloxone (NAL).

FIG. 8 is a panel of graphs and bar graphs showing the effects of adenosine and inosine on PA-I expression.

FIG. 9 is a panel of graphs and bar graphs showing the effects of methylnaltrexone (MNTX) and DAMGO on human endothelial cell barrier regulation.

FIG. 10 is a panel of graphs showing the effects of MNTX effects on non-opioid agonist-induced human endothelial cell barrier regulation.

FIG. 11 is a bar graph showing the differential effects of MNTX and naloxone on agonist-induced human endothelial cell barrier disruption.

FIG. 12 is a panel of bar graphs and immunoblots showing the effects of silencing Mu opioid receptor, S1P1 receptor or S1P3 receptor on MNTX-induced protection from human endothelial cell barrier disruption.

FIG. 13 is a panel of immunoblots and bar graphs showing the effects of MNTX, naloxone and Src on S1P₃ receptor transactivation (tyrosine phosphorylation).

FIG. 14 is a panel of bar graphs showing the analysis (of agonist-induced total cellular tyrosine phosphatase activity in human endothelial cells.

FIG. 15 is a panel of graphs and immunoblots showing the effects of S1P₃ receptor transactivation and endothelial cell barrier function by receptor tyrosine phosphatase mu (RPTPμ).

FIG. 16 is a panel of bar graphs showing the regulation of agonist-induced total cellular tyrosine phosphatase activity and MNTX-induced protection from human endothelial cell barrier disruption by RPTPμ.

FIG. 17 is a panel of immunohistochemical stains and bar graphs showing the effect of MNTX on LPS-induced pulmonary vascular hyper-permeability in vivo.

FIG. 18 is a panel of bar graphs showing the effects of silencing mu opioid receptor expression using siRNA on agonist-induced barrier function.

FIG. 19 is a schematic illustration of pathways relevant to cell barrier function, and dysfunction.

### DETAILED DESCRIPTION

A wide variety of inflammatory disorders, tumor metastasis, and a variety of other diseases and disorders are characterized by a cell barrier dysfunction manifested as an increased cell barrier permeability or loss of selective permeability and concomitant exudation of cells, cellular contents, fluid or protein across the barrier. For example, an endothelial cell barrier dysfunction can lead to increased vascular permeability and a resulting extravasation of protein and fluids, characteristic of inflammatory processes. McVerry et al., Cell. Signal. 17:131-139 (2005). Analogously, a cell barrier dysfunction can become permissive for tumor cell metastasis. An epithelial cell barrier dysfunction arising in the context of, e.g., microbial pathogenesis of the mammalian intestine, can lead to a variety of illnesses, including gut-derived sepsis. Microbial pathogenesis, moreover, can be the product of infection by a pathogen (e.g., Clostridium dificile) or by the phenotypic shift of a normally benign member of the normal flora associated with an organism (e.g., intestinal flora) to a pathogenic or virulent state (e.g., Pseudomonas aeruginosa). Beyond these illustrative examples, multi-cellular organisms such as vertebrates (e.g., mammals, including humans) generally exhibit supracellular compartmentalization resulting in discrete spacings for tissues, organs, and organ systems. Chief contributors to this necessary compartmentalization are the several kinds of cell barriers. Exemplified in terms of endothelial and epithelial cell barriers, there are cell barriers associated with most tissues, organs, and organ systems, e.g., brain (e.g., cerebral endothelial lining/blood brain barrier), spleen, liver, eye, lung, vasculature (blood and lymph), kidney, bladder, ureter, urethra, alimentary canal, including the small and large intestines, lung, and the like. The invention provides methods for preventing, reducing or eliminating a cell barrier dysfunction associated with a disease or disorder that is capable of lowering the quality of life or that deleteriously impacts the health of a subject or patient that has the disease or disorder.

Identification of host stress signaling compounds and the membrane receptors to which they bind, such as receptors on host cells (e.g., epithelial and endothelial cells) as well as receptors on pathogenic microbes such as infectious bacteria, will lead to the discovery of therapeutic targets that will allow for prevention or treatment in a variety of cell barrier diseases and disorders, including the infection, at its most proximate point. Furthermore, the identification of conserved receptors, e.g., bacterial receptors common to other microbial species, will then lead to the development of receptor antagonists or decoys. Such an approach of rendering recipient cells (e.g., colonizing pathogens) insensate to host stress activators has the potential to provide efficacious and cost-effective treatment for a wide variety of diseases and disorders characterized by cell barrier dysfunction.

An “abnormal condition” is broadly defined to include mammalian diseases, mammalian disorders and any abnormal state of mammalian health that is characterized by a cell barrier dysfunction. Exemplary cells that may exhibit a cell barrier dysfunction, or be at risk of developing such a dysfunction, include endothelial cells and epithelial cells. The abnormal conditions may be found in humans, non-human mammals, or any mammal.

“Burn injury” means (i) damage to mammalian tissue resulting from exposure of the tissue to heat, for example in the form of an open flame, steam, hot fluid, and a hot surface.

A “chemical contact injury” refers to an injury caused by direct contact with a chemical and can involve a chemical burn or other injury.

“Severe neutropenia” is given its ordinary and accustomed meaning of a marked decrease in the number of circulating neutrophils.

“Administering” is given its ordinary and accustomed meaning of delivery of a therapeutic to an organism in need by any suitable means recognized in the art. Exemplary forms of administering include delivery by parenteral, oral, subcutaneous, transcutaneous, subcutaneous implantation, intramuscular, intravenous, intrathecal, intraocular, intravitreous, opthalmologic, intraspinal, topical, rectal, transdermal, sublingual, intramuscular, intracavity, and aural routes, as well as by nasal inhalation (e.g., nebulizing spray). The mechanism of delivery may be direct puncture or injection, or gel or fluid application to an eye, ear, nose, mouth, anus or urethral opening, as well as cannulation.

An “effective dose” is that amount of a substance that provides a beneficial effect on the organism receiving the dose and may vary depending upon the purpose of administering the dose, the size and condition of the organism receiving the dose, and other variables recognized in the art as relevant to a determination of an effective dose. The process of determining an effective dose involves routine optimization procedures that are within the skill in the art.

An “animal” is given its conventional meaning of a non-plant, non-protist living being. A preferred animal is a mammal, such as a human.

In the context of the present disclosure, a “need” is an organismal, organ, tissue, or cellular state that could benefit from administration of an effective dose to an organism characterized by that state. For example, a human at risk of developing gut-derived sepsis, or presenting a symptom thereof, is an organism in need of an effective dose of a product, such as a pharmaceutical composition, according to the present invention.

“Average molecular weight” is given its ordinary and accustomed meaning of the arithmetic mean of the molecular weights of the components (e.g., molecules) of a composition, regardless of the accuracy of the determination of that mean. For example, polyethylene glycol, or PEG, having an average molecular weight of 3.5 kilodaltons may contain PEG molecules of varying molecular weight, provided that the arithmetic mean of those molecular weights is determined to be 3.5 kilodaltons at some level of accuracy, which may reflect an estimate of the arithmetic mean, as would be understood in the art. Analogously, PEG 15-20 means PEG whose molecular weights yield an arithmetic mean between 15 and 20 kilodaltons, with that arithmetic mean subject to the caveats noted above. These PEG molecules include, but are not limited to, simple PEG polymers. For example, a plurality of relatively smaller PEG molecules (e.g., 7,000 to 10,000 daltons) may be joined, optionally with a linker molecule such as a phenol, into a single molecule having a higher average molecular weight (e.g., 15,000 to 20,000 daltons).

“PA-I,” or “PA-I lectin/adhesin,” or “PA-IL” expression means the production or generation of an activity characteristic of PA-I lectin/adhesin. Typically, PA-I lectin/adhesin expression involves translation of a PA-I lectin/adhesin-encoding mRNA to yield a PA-I lectin/adhesin polypeptide having at least one activity characteristic of PA-I lectin/adhesin. Optionally, PA-I lectin/adhesin further includes transcription of a PA-I lectin/adhesin-encoding DNA to yield the aforementioned mRNA.

“Intestinal pathogen” means a microbial pathogen capable of causing, in whole or part, gut-derived sepsis in an animal such as a human. Intestinal pathogens known in the art are embraced by this definition, including gram negative bacilli such as the Pseudomonads (e.g., Pseudomonas aeruginosa).

“Pathogenic quorum” means aggregation or association of a sufficient number of pathogenic organisms (e.g., P. aeruginosa) to initiate or maintain a quorum sensing signal or communication that a threshold concentration, or number, of organisms (e.g., intestinal pathogens) are present, as would be known in the art.

“Transcellular Electrical Resistance,” or TER, is given the meaning this phrase has acquired in the art, which refers to a measurement of electrical resistance across cells of a given type (e.g., epithelial or endothelial cells), which is non-exclusively useful in assessing the status of tight junctions between such cells. A related term “TEER,” is used herein to refer to “transepithelial cell electrical resistance,” or “transendothelial cell electrical resistance,” and the particular usage will be apparent from context.

“Pharmaceutical composition” means a formulation of compounds suitable for therapeutic administration, to a living animal, such as a human patient. Preferred pharmaceutical compositions according to the invention are described in the copending U.S. Patent Publication No. 20040266806 the contents of which are herein incorporated herein by reference in their entireties. The pharmaceutical compositions of the invention may comprise a solution balanced in viscosity, electrolyte profile and osmolality, comprising an electrolyte, dextran-coated L-glutamine, dextran-coated inulin, lactulase, D-galactose, N-acetyl D-galactosamine and 5-20% PEG (15,000-20,000). The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entireties.

“Adjuvants,” “carriers,” or “diluents” are each given the meanings those terms have acquired in the art. An adjuvant is one or more substances that serve to prolong the immunogenicity of a co-administered immunogen. A carrier is one or more substances that facilitate the manipulation, such as by translocation of a substance being carried. A diluent is one or more substances that reduce the concentration of, or dilute, a given substance exposed to the diluent.

“Alkyl” refers to an aliphatic hydrocarbon group which is saturated and which may be straight, branched or cyclic and has from 1 to about 10 carbon atoms in the chain, as well as all combinations and subcombinations of chains therein. Exemplary alkyl groups include methyl, ethyl, n-propyl, isopropyl, butyl, isobutyl, sec-butyl, tert-butyl, pentyl, hexyl, heptyl, octyl, nonyl and decyl.

“Lower alkyl” refers to an alkyl group having 1 to about 6 carbon atoms.

“Alkenyl” refers to an aliphatic hydrocarbon group containing at least one carbon-carbon double bond and having from 2 to about 10 carbon atoms in the chain, as well as all combinations and sub-combinations of chains therein. Exemplary alkenyl groups include vinyl, propenyl, butenyl, pentenyl, hexenyl, heptenyl, octenyl, nonenyl and decenyl groups.

“Alkynyl” refers to an aliphatic hydrocarbon group containing at least one carbon-carbon triple bond and having from 2 to about 10 carbon atoms in the chain, as well as combinations and sub-combinations of chains therein. Exemplary alkynyl groups include ethynyl, propynyl, butynyl, pentynyl, hexynyl, heptynyl, octynyl, nonynyl and decynyl groups.

“Alkylene” refers to a bivalent aliphatic hydrocarbon group having from 1 to about 6 carbon atoms, and all combinations and subcombinations of chains therein. The alkylene group may be straight, branched or cyclic. Optionally, there may be inserted within the alkylene group one or more oxygen, sulfur or optionally substituted nitrogen atoms, wherein the nitrogen substituent is an alkyl group, as described previously.

“Alkenylene” refers to an alkylene group containing at least one carbon-carbon double bond. Exemplary alkenylene groups include ethenylene (—CH═CH—) and propenylene (—CH═CHCH₂—).

“Cycloalkyl” refers to any stable monocyclic or bicyclic ring having from about 3 to about 10 carbons, and all combinations and subcombinations of rings therein. Optionally, the cycloalkyl group may be substituted with one or more cycloalkyl-group substituents. Exemplary cycloalkyl groups include cyclopropyl, cyclobutyl, cyclopentyl, cyclohexyl, cycloheptyl and cyclooctyl groups.

“Cycloalkyl-substituted alkyl” refers to a linear alkyl group, preferably a lower alkyl group, substituted at a terminal carbon with a cycloalkyl group, preferably a C3-C8 cycloalkyl group. Exemplary cycloalkyl-substituted alkyl groups include cyclohexylmethyl, cyclohexylethyl, cyclopentylethyl, cyclopentylpropyl, cyclopropylmethyl and the like.

“Cycloalkenyl” refers to an olefinically unsaturated cycloaliphatic group having from about 4 to about 10 carbons, and all combinations and subcombinations of rings therein.

“Alkoxy” refers to an alkyl substituted hydroxyl, or alkyl-O, group, where alkyl is as previously described. Exemplary alkoxy groups include, for example, methoxy, ethoxy, propoxy, butoxy and heptoxy.

“Alkoxy-alkyl” refers to a di-alkyl ether, or alkyl-O-alkyl, group, where alkyl is as previously described.

“Acyl” means an alkyl-CO group wherein alkyl is as previously described. Exemplary acyl groups include acetyl, propanoyl, 2-methylpropanoyl, butanoyl and palmitoyl.

“Aryl” refers to an aromatic carbocyclic group containing from about 6 to about 10 carbons, and all combinations and subcombinations of rings therein. Optionally, the aryl group may be substituted with one or two or more aryl group substituents. Exemplary aryl groups include phenyl and naphthyl.

“Aryl-substituted alkyl” refers to a linear alkyl group, preferably a lower alkyl group, substituted at a terminal carbon with an optionally substituted aryl group, preferably an optionally substituted phenyl ring. Exemplary aryl-substituted alkyl groups include, for example, phenylmethyl, phenylethyl and 3-(4-methylphenyl)propyl.

“Heterocyclic” refers to a monocyclic or multicyclic ring system carbocyclic group or radical containing from about 4 to about 10 members, and all combinations and subcombinations of rings therein, wherein one or more of the members of the ring is an element other than carbon, for example, nitrogen, oxygen or sulfur. The heterocyclic group may be aromatic or nonaromatic. Exemplary heterocyclic groups include, for example, pyrrole and piperidine groups.

“Halo” refers to fluoro, chloro, bromo or iodo.

“Opium alkaloid derivative” refers to mu opioid receptor antagonists (e.g., peripheral antagonists) that are synthetic or semi-synthetic derivatives or analogs of opium alkaloids.

“Substantially no agonist activity,” in connection with the opium alkaloid derivatives, means that, at a concentration of 1 μM, the maximal measured physiological response of a receptor, e.g., electrically stimulated guinea pig ileum, is about 60% or less relative to morphine.

“HMW PEG-like compounds” refer to relatively high molecular weight PEG compounds, defined as having an average molecular weight greater than 3.5 kilodaltons (kD). Preferably, HMW PEG has an average molecular weight greater than 5 kilodaltons and, in particular embodiments, HMW PEG has an average molecular weight at least 8 kilodaltons, more than 12 kilodaltons, at least 15 kilodaltons, and between 15 and 20 kilodaltons. Additionally, “HMW PEG-like compounds includes HMW PEG derivatives wherein each such derivative is an HMW PEG containing at least one additional functional group. Preferred HMW PEG derivatives are cationic polymers. Exemplary functional groups include any of the alkoxy series, preferably C1-C10, any of the aryloxy series, phenyl and substituted phenyl groups. Such functional groups may be attached at any point to an HMW PEG molecule, including at either terminus or in the middle; also included are functional groups, e.g., phenyl and its substituents, that serve to link to smaller PEG molecules or derivative thereof into a single HMW PEG-like compound. Further, the HMW PEG-like molecules having an additional functional group may have one such group or more than one such group; each molecule may also have a mixture of additional functional groups, provided such molecules are useful in stabilizing at least one therapeutic during delivery thereof or in treating, ameliorating or preventing a disease, disorder or condition of an epithelial cell.

“Media” and “medium” are used to refer to cell culture medium and to cell culture media throughout the application. The singular or plural number of the nouns will be apparent from context in each usage.

The term “peripheral” opioid receptor antagonist designates an opioid receptor antagonist, including μ-opioid receptor antagonist, that acts primarily on physiological systems and components external to the central nervous system, i.e., the antagonist does not readily cross the blood-brain barrier. In some embodiments, the peripheral opioid receptor antagonists employed in the methods of the invention exhibit high levels of activity with respect to gastrointestinal tissue, while exhibiting reduced, and preferably substantially no, central nervous system (CNS) activity. The term “substantially no CNS activity,” as used herein, means that less than 20% of the pharmacological activity of the peripheral opioid receptor antagonists exhibited outside the CNS is exhibited inside the CNS. In preferred embodiments, the peripheral opioid receptor antagonists employed in the inventive methods exhibit less than 15% of their pharmacological activity in the CNS, with less than about 10% being more preferred. In even more preferred embodiments, the peripheral opioid receptor antagonists employed in the methods of the invention exhibit less than 5% of their pharmacological activity in the CNS, with about 0% (i.e., no CNS activity) also being more preferred. Preferred peripheral opioid receptor antagonists of the invention are quaternary derivatives of noroxymorphone, such as R-methylnaltrexone.

In general terms, a model of lethal sepsis in mice has been developed which provides unique insight into the process by which microbial pathogens can cause lethal sepsis syndrome from within the intestinal tract of a physiologically stressed host. Three physiologic “hits” result in mortality, e.g., surgical stress (30% hepatectomy), starvation (48 hour of water only) and the introduction of P. aeruginosa into the distal intestinal tract (cecum). This model results in 100% mortality, whereas elimination of any one of the three factors results in complete survival. A single virulence determinant has been identified in Pseudomonas aeruginosa, PA-I, that is expressed in vivo in response to locally released compounds unique to the intestinal tract of a physiologically stressed host. That PA-I plays a role in lethal gut-derived sepsis, such as in mice, was demonstrated by experiments in which mutanized strains of P. aeruginosa, void of PA-I yet capable of secreting exotoxin A, had markedly attenuated effects on the barrier function of cultured epithelial cells and were completely apathogenic in the mouse model of lethal gut-derived sepsis. PA-I lectin/adhesin plays a key role in the lethal effect of this organism by creating a permeability defect to potent and lethal cytotoxins of P. aeruginosa, such as exotoxin A and elastase. The lethal effect of intestinal P. aeruginosa appears to occur completely independent of its extraintestinal dissemination (translocation). Surprisingly, systemic injection (intravenous, intraperitoneal) of an equal dose of P. aeruginosa in this model produces no mortality and no systemic inflammation. Taken together, the data provide strong evidence that sepsis can be generated by a microbial pathogen whose virulence is activated locally by host stress-derived bacterial signaling compounds (BSC) generated during surgical stress.

Observation that P. aeruginosa is much more virulent and lethal when present on an epithelial surface than when bloodborne is supported by a lung model of sepsis. Intravenous injection of a highly cytotoxic strain of P. aeruginosa, PA103, resulted in no systemic cytokine release and no mortality in rabbits, whereas lung instillation of an equal dose (approximately 10⁸ cfu/ml) resulted in significant systemic cytokine release (TNFα, IL-8) and 100% mortality. An extensive number of studies have now demonstrated that the most virulent and lethal strains of P. aeruginosa causing sepsis following lung instillation are not those that display the most invasive (translocating/disseminating) phenotype, but rather are those strains that are most disruptive of cellular integrity and epithelial permselectivity to its locally released cytotoxins. These observations, coupled with the findings that P. aeruginosa produces a 25-fold increase in its extracellular virulence factors (i.e., elastase, alkaline protease) when cultured in the presence of epithelial cells, suggests that the lethality of this pathogen is governed by its interaction with, and activation by, the epithelium itself. Experimental data show that both soluble and contact-mediated elements of the intestinal epithelium exposed to stress (e.g., surgery, hypoxia, heat shock), enhance the capacity of P. aeruginosa to express PA-I, which is capable of causing a profound disruption in the cellular integrity of both intestinal and lung epithelial cells.

The main mechanism of action by which P. aeruginosa induces mortality from within the intestinal tract of a stressed host is via a PA-1-induced permeability defect to its lethal cytotoxins, exotoxin A and elastase. Instillation of a combination of purified PA-I with either exotoxin A or elastase into the cecum of surgically stressed and sham-operated control mice induced significant mortality, whereas injection of either compound alone had no effect. The clinical role of PA-I was examined by screening fecal samples of patients with severe sepsis for whom no source could be identified. Among strains of P. aeruginosa isolated from the feces of critically ill patients, as well as among numerous laboratory and environmental strains, the PA-I genotype has been found to be highly prevalent. There is now convincing evidence that the intestinal tract environment is a unique niche in which key virulence determinants in highly lethal pathogens (i.e., Vibrio cholera) are activated by yet-unknown “cues” that are present only during active infection.

The gene encoding PA-I (the lecA gene) is an ideal biological “read-out” and reporter gene in which to examine overall virulence gene expression in P. aeruginosa in response to host stress-derived BSCs.

The precise host cell elements that activate bacterial biosensors are not known. Because PA-I expression is both QS and RpoS dependent, GFP-PA-I reporter strains (described herein) provide a unique opportunity to screen for host cell-derived bacterial signaling compounds released during stress that activate membrane sensors, leading to PA-I expression.

Various opioid receptor agonists, including endogenous morphine alkaloids, are released and maintained at sustained concentrations during severe stress. Opioids are highly conserved compounds and various bacteria and fungi, including P. aeruginosa, synthesize and metabolize morphine. Similarly, as shown herein, elements of the immune system, such as IFN-γ, can also serve as potent host stress-derived-BSCs. P. aeruginosa is able to sense the presence of the IFN-γ and respond by expressing two quorum sensing dependent virulence factors, PA-I and pyocyanin. From the perspective of P. aeruginosa, the ability to sense and respond to host immune activation, in particular to IFN-γ whose function is directed at bacterial clearance, provides this organism with a countermeasure against host immune activation. In particular, Interferon-γ is shown below to bind to an outer membrane protein in P. aeruginosa, OprF, resulting in the expression of a quorum sensing-dependent virulence determinant, the PA-I lectin. IFN-γ also bound E. coli membranes. These observations provide details of the mechanisms by which prokaryotic organisms are directly signaled by immune activation in their eukaryotic host.

Exposure of P. aeruginosa to opioids leads to the expression of several quorum sensing-dependent virulence factors in P. aeruginosa. That the QS system might be activated by opioids is a significant finding given that QS controls the expression of hundreds of virulence genes in P. aeruginosa (M. Schuster, M. L. Urbanowski and E. P. Greenberg, Proc Natl Acad Sci USA 101, 15833 (2004)).

Data disclosed herein provide evidence that MvfR is required for PCN production in response to U-50,488. In addition, data from the present study suggest that PCN production in response to U-50,488 also requires the synthesis of Pseudomonas quinolone signal (PQS), since methyl anthranilate attenuated the U-50,488-mediated effect on PCN production. That C4-HSL also requires intact MvfR to produce PCN, coupled with the finding of highly up-regulated PCN production in strains harboring multiple mvfR genes, is consistent with quorum sensing activation relying not only on the binding of QS signaling molecules to their core QS transcriptional regulators (i.e., RhIR, LasR), but also having QS signals activating proximal transcriptional regulators.

The data disclosed herein establish that opioid compounds may vary in their ability to induce a particular virulence phenotype in P. aeruginosa. It is contemplated that there are multiple host-stress-derived bacterial signaling compounds that are able to influence the state of virulence in P. aeruginosa. Norepinephrine can also affect the QS-dependent virulence factor PA-IL in P. aeruginosa (J. Alverdy, et al., Ann Surg 232, 480 (2000)) and soluble compounds released into the media by hypoxic intestinal epithelial cells also induce PA-IL expression. Consistent with these disclosures is the disclosure that norepinephrine directly affects QS circuitry in E. coli (V. Sperandio, A. G. Torres, B. Jarvis, J. P. Nataro and J. B. Kaper, Proc Natl Acad Sci USA 100, 8951 (2003)).

The invention provides methods of screening for modulators of the signaling induced by one or more BSCs, including such modulators as opioid receptor agonists, morphine, and interferon gamma. These therapeutics are delivered to an organism, such as a human patient, in need thereof. Dosage levels and delivery routes and schedules will vary depending upon circumstances readily identified and accommodated by those skilled in the art using routine procedures.

The therapeutics according to the invention may further comprise a HMW PEG-like compound, which may be administered by any means suitable for the condition or disorder to be treated. The compound(s) may be delivered orally, such as in the form of tablets, capsules, granules, powders, or with liquid formulations including syrups; by sublingual; buccal; or transdermal delivery; by injection or infusion parenterally, subcutaneously, transcutaneously, subcutaneous implantation, intravenously, intramuscularly, intrathecally, intraocularly, opthalmologically, intraspinally, topically, or intrasternally (e.g., as sterile injectable aqueous or non-aqueous solutions or suspensions); orally, nasally, such as by inhalation spray; aurally, rectally such as in the form of suppositories; vaginally or urethrally via suppository or infusion, e.g., via cannulation, or liposomally, and intracavity delivery. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions known in the art.

Exemplary compositions for oral administration include suspensions which may contain, for example, microcrystalline cellulose for imparting bulk, alginic acid or sodium alginate as a suspending agent, methylcellulose as a viscosity enhancer, sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants, such as those known in the art. The inventive compounds may be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be excipients such as a relatively high molecular weight cellulose (AVICEL®) or a polyethylene glycol (PEG; GoLytely®, 3.34 kD); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for intestinal administration include solutions or suspensions which may contain, for example, suitable non-toxic diluents or solvents, such as mannitol, 1,3-butanediol, water, Ringer's solution, an isotonic sodium chloride solution, or other suitable dispersing or wetting and suspending agents, including synthetic mono- or diglycerides and fatty acids, including oleic acid. Contemplated in this context are suppositories which may contain, for example, suitable non-irritating excipients, such as cocoa buffer, synthetic glyceride esters or polyethylene glycols (e.g., GoLytely®).

The effective amount of a compound of the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like, at risk of developing a microbe-mediated epithelial condition or disease, such as gut-derived sepsis, or at risk of developing an inflammatory disorder, e.g., acute lung injury, characterized by cell barrier dysfunction. Generally, the peripheral opioid receptor antagonists of the invention are administered in an effective amount, e.g., from 10⁻⁶ M to 10⁻⁹ M. Patient drug plasma levels may be measured using routine HPLC methods known to those of skill in the art.

The invention provides methods of administering opioid receptor antagonists to treat, prevent, or alleviate a symptom associated with, a disease or disorder characteristically exhibiting a cell barrier dysfunction. The opioid receptor antagonist may be a mu opioid antagonist, or the antagonist may be a kappa opioid antagonist. The invention also encompasses administration of more than one opioid antagonist, including combinations of mu antagonists, combinations of kappa antagonists, and combinations of at least one mu antagonist and at least one kappa antagonist; the invention further comprehends administration of combinations of at least one centrally acting opioid receptor antagonist and at least one peripherally restricted opioid receptor antagonist. For example, a combination of methylnaltrexone and either alvimopan or its metabolite ADL 08-0011, or a combination of naltrexone and methylnaltrexone, may be administered.

As described below in the examples, and in particular Example 26, it has also been found that both morphine and DAMGO induce cell barrier dysfunction, such as pulmonary microvascular endothelial cell barrier disruption. Communication between blood and tissue occurs through the delivery of molecules and circulating substances across the endothelial barrier by directed transport either through or between cells. Certain inflammatory syndromes, for example, acute lung injury and sepsis, reduce barrier function. Such barrier disruption results in increased vascular permeability and organ dysfunction. Disclosed below are data establishing that a peripheral opioid receptor antagonist in accordance with the invention enhanced endothelial cell barrier function. Specifically, the cell barrier disruption is blocked by pretreatment with a peripheral opioid receptor antagonist. For example, pretreatment with a peripheral opioid receptor antagonist (e.g., MNTX) protects against cell barrier dysfunction arising from either μ opioid receptor-dependent or μ opioid receptor-independent effects. Of course, the peripheral opioid receptor antagonist is also useful in protecting against cell barrier dysfunction arising from both μ opioid receptor-dependent effects, e.g., effects of μ opioid receptor agonist (e.g., morphine) binding, and R opioid receptor-independent effects, e.g., effects realized without a contribution from a μ opioid receptor, such as thrombin- and/or lipopolysaccharide (LPS)-dependent cell barrier dysfunction or disruption, such as in endothelial cells. Thus, μ opioid receptor antagonists, e.g., peripheral μ opioid receptor antagonists, are useful in the prevention or treatment of inflammatory syndromes, e.g., acute lung injury, atherosclerosis, and other diseases characterized by a cell barrier dysfunction. Thus, the methods of the invention have therapeutic value in the treatment of those syndromes characterized by barrier dysfunction or disruption, e.g., atherosclerosis, acute lung injury, microbial infection, and the like. It is, therefore, contemplated that the invention includes methods of reducing cell barrier disruption by administering to the cells an effective amount of a cell barrier enhancement protective agent, e.g., MNTX.

The methods of the invention also encompass treating patients who are undergoing treatment with opioid receptor agonists, although in some embodiments, the patients are not chronic recipients of any opioid receptor agonist. The opioid receptor agonists may be exogenously or endogenously supplied, and the agonist may be a naturally occurring opioid or a non-naturally occurring synthetic compound. As but one example of a method of treating a patient undergoing treatment with an opioid receptor agonist, cancer patients frequently receive morphine to manage pain associated with advanced stages of the disease and, while the μ opioid receptor antagonists are useful in this context in providing beneficial effects on cell barrier dysfunction without undermining efforts to manage pain, these μ opioid receptor antagonists also find use in treating cancer at a much earlier stage. In particular, the μ opioid receptor antagonists are beneficially administered to cancer patients having pre-metastatic stage tumors, e.g., peri-operatively, where pain management may not dictate the need for a μ opioid receptor agonist such as morphine. At this relatively early stage in the progression of many cancers, a μ opioid receptor antagonist provides therapeutic support of normal cell barrier function, facilitating resistance to the metastatic processes (i.e., tumor cell seeding) that exploit cell barrier dysfunction. Consequently, μ opioid receptor antagonists have a particular application in pre-metastatic cancer patient populations, which are populations typically free of chronic recipients of opioid receptor agonists like morphine. In a particular embodiment of this aspect of the invention, a μ opioid receptor antagonist, e.g., a peripheral μ opioid receptor antagonist such as MNTX, is administered intra- or peri-operatively during cancer surgery. It is expected that any type of cancer amenable to surgery will be amenable to peri-operative administration of a μ opioid receptor antagonist. Without wishing to be bound by theory, the surgical intervention creates a host stress that may signal cells, such as endothelial and/or epithelial cells of a wide variety of tissues, organs and organ systems (e.g., lung, gut, vasculature, eye) in a manner that leads to a cell barrier dysfunction that facilitates cancer cell mobilization or metastasis. Indirect evidence in support of this non-binding theory is available in a retrospective study of breast cancer patients undergoing surgery. Exploration of “surgical stress” led to a comparative study of regional anesthesia, in the form of paravertebral anesthesia (levobupivacaine), versus post-operative morphine analgesia for the surgical patients. The results showed a substantial reduction in tumor recurrence and metastasis when regional anesthesia was administered rather than post-operative morphine. The results are consistent with the view that the difference in outcomes was attributable to the deleterious effect of morphine rather than the beneficial effect of regional anesthetics. Thus, any agent, such as opioid receptor antagonists, including peripheral opioid receptor antagonists, that counteracts the effects of morphine would be beneficial in the peri-operative environment of cancer surgery, regardless of whether an opioid agonist such as morphine were contemplated as part of the surgical treatment or post-operative care protocol.

Opioid receptor agonists include, but are not limited to, morphine, methadone, codeine, meperidine, fentidine, fentanil, sufentanil, alfentanil and the like. Opioid receptor agonists are classified by their ability to agonize one type of receptor an order of magnitude more effectively than another. For example, the relative affinity of morphine for the mu receptor is 200 times greater than for the kappa receptor, and it is therefore classified as a mu opioid receptor agonist. Some opioid compounds may act as agonists towards one receptor type and as antagonists toward another receptor type; such and are classified as agonist/antagonists, (also known as mixed or partial agonists). “Agonist/antagonist,” “partial agonist,” and “mixed agonist” are used interchangeably herein. These opioids include, but are not limited to, pentazocine, butorphanol, nalorphine, nalbufine, buprenorphine, bremazocine, and bezocine. Many of the agonist/antagonist group of opioids are agonists of the kappa receptors and antagonists of the mu receptors. Further, it is envisioned that the active metabolites of opioid receptor agonists will also be active in the methods of the invention. For example, the metabolites of morphine, morphine 3-glucuronide and morphine 6-glucuronide, are expected to be active in preventing, reducing or eliminating cell barrier dysfunction.

The ability to selectively antagonize peripheral opioid receptors to avoid, e.g., unacceptable interference with patient pain management indicates that peripheral opioid receptor antagonists will be useful in addressing cell barrier dysfunction-related diseases and disorders. The peripheral opioid receptor antagonists form a class of compounds that can vary in structure while maintaining the restriction to peripheral receptor interaction. These compounds include tertiary and quaternary morphinans, in particular noroxymorphone derivatives, N-substituted piperidines, and in particular, piperidine-N-alkylcarboxylates, and tertiary and quaternary benzomorphans. Peripherally restricted antagonists, while varied in structure, are typically charged, polar and/or of high molecular weight, each of which impedes crossing of the blood-brain barrier.

Examples of opioid receptor antagonists that cross the blood-brain barrier and are centrally (and peripherally) active include, e.g., naloxone, naltrexone (each of which is commercially available from Baxter Pharmaceutical Products, Inc.) and nalmefene (available, e.g., from DuPont Pharma). These may be of value in attenuating cell barrier dysfunction in certain patients, such as those not being treated for pain management or other opiate treatment.

In certain embodiments, the present methods involve the administration to a patient of a peripheral μ-opioid receptor antagonist that is a piperidine-N-alkylcarboxylate compound. Piperidine-N-alkylcarboxylate opioid antagonists include, for example, the compounds disclosed in U.S. Pat. Nos. 5,250,542; 5,159,081; 5,270,328; and 5,434,171, the disclosures of which are hereby incorporated herein by reference, in their entireties. A class of piperidine-N-alkylcarboxylate opioid antagonists include those having the following formula (I):

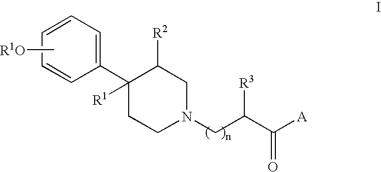

wherein:
R1 is hydrogen or alkyl;
R2 is hydrogen, alkyl or alkenyl;
R3 is hydrogen, alkyl, alkenyl, aryl, cycloalkyl, cycloalkenyl, cycloalkyl-substituted alkyl, cycloalkenyl-substituted alkyl or aryl-substituted alkyl;
R4 is hydrogen, alkyl or alkenyl;
A is OR5 or NR6 R7; wherein:
R5 is hydrogen, alkyl, alkenyl, cycloalkyl, cycloalkenyl, cycloalkyl-substituted alkyl, cycloalkenyl-substituted alkyl, or aryl-substituted alkyl;
R6 is hydrogen or alkyl;
R7 is hydrogen, alkyl, alkenyl, cycloalkyl, aryl, cycloalkyl-substituted alkyl, cycloalkenyl, cycloalkenyl-substituted alkyl, aryl-substituted alkyl, aryl-substituted alkyl, or alkylene substituted B or, together with the nitrogen atom to which they are attached, R6 and R7 form a heterocyclic ring; B is

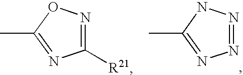

C(═O)W or NR8 R9; wherein;
R8 is hydrogen or alkyl;
R9 is hydrogen, alkyl, alkenyl, cycloalkyl-substituted alkyl, cycloalkyl, cycloalkenyl, cycloalkenyl-substituted alkyl, aryl or aryl-substituted alkyl or, together with the nitrogen atom to which they are attached, R8 and R9 form a heterocyclic ring;
W is OR10, NR11 R12, or OE; wherein
R10 is hydrogen, alkyl, alkenyl, cycloalkyl, cycloalkenyl, cycloalkyl-substituted alkyl, cycloalkenyl-substituted alkyl, or aryl-substituted alkyl;
R11 is hydrogen or alkyl;
R12 is hydrogen, alkyl, alkenyl, aryl, cycloalkyl, cycloalkenyl, cycloalkyl-substituted alkyl, cycloalkenyl-substituted alkyl, aryl-substituted alkyl or alkylene substituted C(═O)Y or, together with the nitrogen atom to which they are attached, R11 and R12 form a heterocyclic ring;

### E is

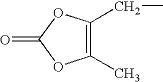

alkylene substituted (C═O)D, or —R13OC(═O)R14;
wherein
R13 is alkyl substituted alkylene;
R14 is alkyl;

### D is OR15 or NR16 R17;

wherein:
R15 is hydrogen, alkyl, alkenyl, cycloalkyl, cycloalkenyl, cycloalkyl-substituted alkyl, cycloalkenyl-substituted alkyl, or aryl-substituted alkyl;
R16 is hydrogen, alkyl, alkenyl, aryl, aryl-substituted alkyl, cycloalkyl, cycloalkenyl, cycloalkyl-substituted alkyl or cycloalkenyl-substituted alkyl;
R17 is hydrogen or alkyl or, together with the nitrogen atom to which they are attached, R16 and R17 form a heterocyclic ring;

### Y is OR18 or NR19 R20;

wherein:
R18 is hydrogen, alkyl, alkenyl, cycloalkyl, cycloalkenyl, cycloalkyl-substituted alkyl, cycloalkenyl-substituted alkyl, or aryl-substituted alkyl;
R19 is hydrogen or alkyl;
R20 is hydrogen, alkyl, alkenyl, aryl, cycloalkyl, cycloalkenyl, cycloalkyl-substituted alkyl, cycloalkenyl-substituted alkyl, or aryl-substituted alkyl or, together with the nitrogen atom to which they are attached, R19 and R20 form a heterocyclic ring;
R21 is hydrogen or alkyl; and
n is 0 to about 4;
or a stereoisomer, prodrug, or pharmaceutically acceptable salt, hydrate or N-oxide thereof.

In the above formula (I), R1 is hydrogen or alkyl. In some embodiments, R1 is hydrogen or C1-C5 alkyl. In important embodiments, R1 is hydrogen.

In the above formula (I), R2 is hydrogen, alkyl or alkenyl. In some embodiments, R2 is hydrogen, C1-C5 alkyl or C2-C6 alkenyl. In some embodiments, R2 is alkyl, with C1-C3 alkyl being more preferred.

In the above formula (I), R3 is hydrogen, alkyl, alkenyl, aryl, cycloalkyl, cycloalkenyl, cycloalkyl-substituted alkyl, cycloalkenyl-substituted alkyl or aryl-substituted alkyl. In some embodiments, R3 is hydrogen, C1-C10 alkyl, C3-C10 alkenyl, phenyl, cycloalkyl, C5-C8 cycloalkenyl, cycloalkyl-substituted C1-C3 alkyl, C5-C8 cycloalkyl-substituted C1-C3 alkyl or phenyl-substituted C1-C3 alkyl. In some embodiments, R3 is benzyl, phenyl, cyclohexyl, or cyclohexylmethyl.

In the above formula (I), R4 is hydrogen, alkyl or alkenyl. In some embodiments, R4 is hydrogen, C1-C5 alkyl or C2-C6 alkenyl. In other embodiments, R4 is C1-C3 alkyl, with methyl being more preferred.

In the above formula (I), A is OR5 or NR6 R7.

In the above formula (I), R5 is hydrogen, alkyl, alkenyl, cycloalkyl, cycloalkenyl, cycloalkyl-substituted alkyl, cycloalkenyl-substituted alkyl, or aryl-substituted alkyl. In some embodiments, R5 is hydrogen, C₁-C10 alkyl, C2-C10 alkenyl, cycloalkyl, C5-C8 cycloalkenyl, cycloalkyl-substituted C1-C3 alkyl, C5-C8 cycloalkenyl-substituted C1-C3 alkyl, or phenyl-substituted C1-C3 alkyl. Also in some embodiments, R5 is hydrogen or alkyl, with C1-C3 alkyl being more preferred.

In the above formula (I), R6 is hydrogen or alkyl. In some embodiments, R6 is hydrogen or C1-C3 alkyl. In some embodiments, R6 is hydrogen.

In the above formula (I), R7 is hydrogen, alkyl, alkenyl, cycloalkyl, aryl, cycloalkyl-substituted alkyl, cycloalkenyl, cycloalkenyl-substituted alkyl, aryl-substituted alkyl, aryl-substituted alkyl or alkylene substituted B. In some embodiments, R7 is hydrogen, C1-C10 alkyl, C3-C10 alkenyl, phenyl, cycloalkyl, cycloalkyl-substituted C1-C3 alkyl, C5-C8 cycloalkenyl, C5-C8 cycloalkenyl-substituted C1-C3 alkyl, phenyl-substituted C1-C3 alkyl or (CH2)_{q}-B. In some embodiments, R7 is (CH2)_{q}-B.

In certain alternative embodiments, in the above formula (I), R6 and R7 form, together with the nitrogen atom to which they are attached, a heterocyclic ring.

The group B in the definition of R7 is

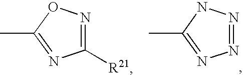

C(═O)W or NR8 R9. In some embodiments, B is C(═O)W.

The group R8 in the definition of B is hydrogen or alkyl. In some embodiments, R8 is hydrogen or C1-C3 alkyl.

The group R9 in the definition of B is hydrogen, alkyl, alkenyl, cycloalkyl-substituted alkyl, cycloalkyl, cycloalkenyl, cycloalkenyl-substituted alkyl, aryl or aryl-substituted alkyl. In some embodiments, R9 is hydrogen, C1-C10 alkyl, C3-C10 alkenyl, cycloalkyl-substituted C1-C3 alkyl, cycloalkyl, C5-C8 cycloalkenyl, C5-C8 cycloalkenyl-substituted C1-C3 alkyl, phenyl or phenyl-substituted C1-C3 alkyl.

In certain alternative embodiments, in the definition of B, R8 and R9 form, together with the nitrogen atom to which they are attached, a heterocyclic ring.

The group W in the definition of B is OR10, NR11 R12 or OE.

The group R10 in the definition of W is hydrogen, alkyl, alkenyl, cycloalkyl, cycloalkenyl, cycloalkyl-substituted alkyl, cycloalkenyl-substituted alkyl, or aryl-substituted alkyl. In some embodiments, R10 is hydrogen, C1-C10 alkyl, C2-C10 alkenyl, cycloalkyl, C5-C8 cycloalkenyl, cycloalkyl-substituted C1-C3 alkyl, C5-C8 cycloalkenyl-substituted C1-C3 alkyl, or phenyl-substituted C1-C3 alkyl. Also in some embodiments, R10 is hydrogen, alkyl, C1-C5 alkyl, phenyl-substituted alkyl, phenyl-substituted C1-C2 alkyl, cycloalkyl or cycloalkyl-substituted alkyl, C5-C6 cycloalkyl-substituted C1-C3 alkyl.

The group R11 in the definition of W is hydrogen or alkyl. In some embodiments, R11 is hydrogen or C1-C3 alkyl.

The group R12 in the definition of W is hydrogen, alkyl, alkenyl, aryl, cycloalkyl, cycloalkenyl, cycloalkyl-substituted alkyl, cycloalkenyl-substituted alkyl, aryl-substituted alkyl or alkylene-substituted C(═O)Y. In some embodiments, R12 is hydrogen, C1-C10 alkyl, C3-C10 alkenyl, phenyl, cycloalkyl, C5-C8 cycloalkenyl, cycloalkyl-substituted C1-C3 alkyl, C5-C8 cycloalkenyl-substituted C1-C3 alkyl, phenyl-substituted C1-C3 alkyl, or alkylene-substituted C(═O)Y. Also in some embodiments, R12 is hydrogen, alkyl, some C1-C3 alkyl or (CH2)m C(O)Y, where m is 1 to 4.

The group Y in the definition of R12 is OR18 or NR19 R20.

In certain alternative embodiments, in the definition of W, R12 and R13 form, together with the nitrogen atom to which they are attached, a heterocyclic ring.

The group E in the definition of W is:

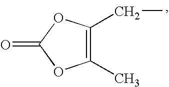

alkylene substituted (C═O)D, or —R13OC(═O)R14. In some embodiments, E is:

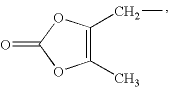

(CH2)m (C═O)D (where m is as defined above), or —R13OC(═O)R14.

The group R13 in the definition of E is alkyl substituted alkylene. In some embodiments, R13 is C1-C3 alkyl substituted methylene. In some embodiments, R13 is —CH(CH3)- or —CH(CH2 CH3)-.

The group R14 in the definition of E is alkyl. In some embodiments, R14 is C1-C10 alkyl.

The group D in the definition of E is D is OR15 or NR16 R17.

The group R15 in the definition of D is hydrogen, alkyl, alkenyl, cycloalkyl, cycloalkenyl, cycloalkyl-substituted alkyl, cycloalkenyl-substituted alkyl, or aryl-substituted alkyl. In some embodiments, R15 is hydrogen, C1-C10 alkyl, C2-C10 alkenyl, cycloalkyl, C5-C8 cycloalkenyl, cycloalkyl-substituted C1-C3 alkyl, C5-C8 cycloalkenyl-substituted C1-C3 alkyl, or phenyl-substituted C1-C3 alkyl. Also in some embodiments, R15 is hydrogen or alkyl, with C1-C3 alkyl being more preferred.

The group R16 in the definition of D is hydrogen, alkyl, alkenyl, aryl, aryl-substituted alkyl, cycloalkyl, cycloalkenyl, cycloalkyl-substituted alkyl or cycloalkenyl-substituted alkyl. In some embodiments, R16 is hydrogen, C1-C10 alkyl, C3-C10 alkenyl, phenyl, phenyl-substituted C1-C3 alkyl, cycloalkyl, C5-C8 cycloalkenyl, cycloalkyl-substituted C1-C3 alkyl, C5-C8 cycloalkenyl-substituted C1-C3 alkyl. In some embodiments, R16 is methyl or benzyl.

The group R17 in the definition of D is hydrogen or alkyl. In some embodiments, R17 is hydrogen or C1-C3 alkyl. In even more some embodiments, R17 is hydrogen.

In certain alternative embodiments, in the definition of D, R16 and R17 form, together with the nitrogen atom to which they are attached, a heterocyclic ring.

The group R18 in the definition of Y is hydrogen, alkyl, alkenyl, cycloalkyl, cycloalkenyl, cycloalkyl-substituted alkyl, cycloalkenyl-substituted alkyl, or aryl-substituted alkyl. In some embodiments, R18 is hydrogen, C1-C10 alkyl, C2-C10 alkenyl, cycloalkyl, C5-C8 cycloalkenyl, cycloalkyl-substituted C1-C3 alkyl, C5-C8 cycloalkenyl-substituted C1-C3 alkyl, or phenyl-substituted C1-C3 alkyl. In some embodiments, R18 is hydrogen or C1-C3 alkyl.

The group R19 in the definition of Y is hydrogen or alkyl. In some embodiments, R19 is hydrogen or C1-C3 alkyl.

The group R20 in the definition of Y is hydrogen, alkyl, alkenyl, aryl, cycloalkyl, cycloalkenyl, cycloalkyl-substituted alkyl, cycloalkenyl-substituted alkyl, or aryl-substituted alkyl. In some embodiments, R20 is hydrogen, C1-C10 alkyl, C3-C10 alkenyl, phenyl, cycloalkyl, C5-C8 cycloalkenyl, cycloalkyl-substituted C1-C3 alkyl, C5-C₈ cycloalkenyl-substituted C1-C3 alkyl, or phenyl-substituted C1-C3 alkyl. In some embodiments, R20 is hydrogen or C1-C3 alkyl.

In certain alternative embodiments, in the definition of Y, R19 and R20 form, together with the nitrogen atom to which they are attached, a heterocyclic ring.

The group R21 in the definition of B is hydrogen or alkyl. In some embodiments, R21 is hydrogen or C1-C3 alkyl. In some embodiments, R1 is hydrogen.

In the above formula (I), n is 0 to about 4. In some embodiments, n is about 1 or 2.

In the above definition of R7, q is about 1 to about 4. In some embodiments, q is about 1 to about 3.

In the above definition of E, m is about 1 to about 4. In some embodiments, m is about 1 to about 3.

The compounds of formula (I) can occur as the trans and cis stereochemical isomers by virtue of the substituents at the 3- and 4-positions of the piperidine ring, and such stereochemical isomers are within the scope of the claims. The term “trans”, as used herein, refers to R2 in position 3 being on the opposite side from the methyl group in position 4, whereas in the “cis” isomer R2 and the 4-methyl are on the same side of the ring. In the methods of the present invention, the compounds employed may be the individual stereoisomers, as well as mixtures of stereoisomers. In some embodiments, the methods of the present invention involve compounds of formula (I) wherein the group R2 at the 3-position is situated on the opposite side of the ring, i.e., trans to the methyl group in the 4-position and on the same side of the ring. These trans isomers can exist as the 3R,4R-isomer, or the 3S,4S-isomer.

The terms “R” and “S” are used herein as commonly used in organic chemistry to denote specific configuration of a chiral center. The term “R” refers to “right” and refers that configuration of a chiral center with a clockwise relationship of group priorities (highest to second lowest) when viewed along the bond toward the lowest priority group. The term “S” or “left” refers to that configuration of a chiral center with a counterclockwise relationship of group priorities (highest to second lowest) when viewed along the bond toward the lowest priority group. The priority of groups is based upon their atomic number (heaviest isotope first). A partial list of priorities and a discussion of stereochemistry is contained in the book: The Vocabulary of Organic Chemistry, Orchin, et al., John Wiley and Sons Inc., page 126 (1980), which is incorporated herein by reference in its entirety.

Piperidine-N-alkylcarboxylate compounds for use in the methods of the present invention are those of formula (I) in which the configuration of substituents on the piperidine ring is 3R and 4R.

When R3 is not hydrogen, the carbon atom to which R3 is attached is asymmetric. As such, this class of compounds can further exist as the individual R or S stereoisomers at this chiral center, or as mixtures of stereoisomers, and all are contemplated within the scope of the present invention. A substantially pure stereoisomer of the compounds of this invention can be used, i.e., an isomer in which the configuration at the chiral center to which R3 is attached is R or S, i.e., those compounds in which the configuration at the three chiral centers is 3R, 4R, 5 or 3R, 4R, R.

Furthermore, other asymmetric carbons can be introduced into the molecule depending on the structure of A. As such, these classes of compounds can exist as the individual R or S stereoisomers at these chiral centers, or as mixtures of stereoisomers, and all are contemplated as being within the scope of methods of the present invention.

Certain piperidine-N-alkylcarboxylate compounds for use in the methods of the present invention include the following:

U—OCH2 CH3; U—OH; G—OH; U—NHCH2 C(O)NHCH3; U—NHCH2 C(O)NH2; G—NHCH2 C(O)NHCH3; U—NHCH2 C(O)NHCH2 CH3; G-NH(CH2)3 C(O)OCH2 CH3; G—NHCH2 C(O)OH; M—NHCH2 C(O)NH2; M—NH(CH2)2 C(O)OCH2 (C6H5); X—OCH2 CH3; X—OH; X—NH(CH2)2 CH3; Z-NH(CH2)3 C(O)OCH2 CH3; X—NHCH2 C(O)OH; Z-NH(CH2)2 N(CH3)2; Z-NH(CH2)2 C(O)NHCH2 CH3; X—OCH2 (C6H5); X—N(CH3)2; Z-NH(CH2)3 C(O)NHCH3; Z-NH(CH2)3 C(O)NH2; Z-NH(CH2)3 C(O)NHCH2 CH3; X—OCH2 C(O)OCH3; X—OCH2 C(O)NHCH3; and X—N(CH3)CH2 C(O)CH2 CH3; in which:

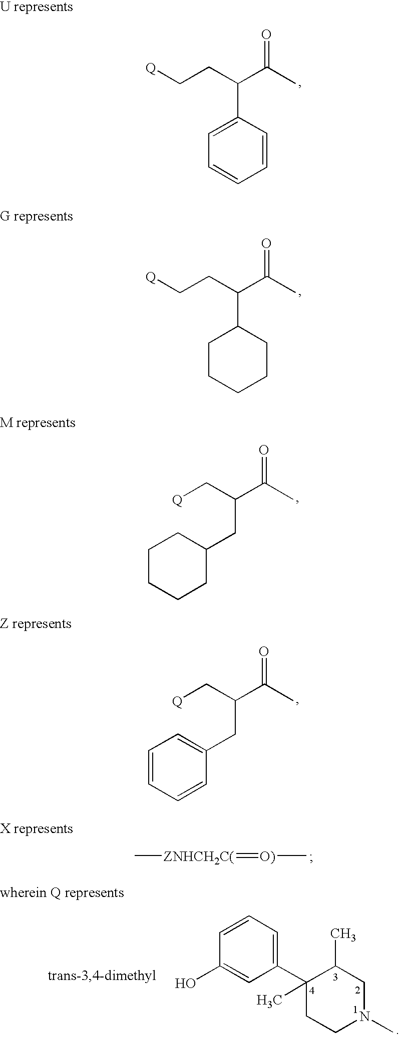

Important piperidine-N-alkylcarboxylate compounds for use in the methods of the present invention include the following:

Z—OH; Z-NH(CH2)2 C(O)OH; G-NH(CH2)2 C(O)NH2; G-NH(CH2)2 C(O)NHCH3; G—NHCH2 C(O)NH2; G-NHCH2 C(O)NHCH2 CH3; G-NH(CH2)3 C(O)NHCH3; G—NH(CH2)2 C(O)OH; G-NH(CH2)3 C(O)OH; X—NH2; X—NHCH(CH3)2; X—OCH2 CH(CH3)2; X—OCH2 C6H5; X—OH; X—O(CH2)4 CH3; X—O—(4-methoxycyclohexyl); X—OCH(CH3)OC(O)CH3; X—OCH2 C(O)NHCH2 (C6H5); M—NHCH2 C(O)OH; M—NH(CH2)2 C(O)OH; M—NH(CH2)2 C(O)NH2; U-NHCH2 C(O)OCH2 CH3; and U—NHCH2 C(O)OH; wherein Z, G, X, M and U are as defined above.

Stated another way, in accordance with some embodiments of the invention, the compound of formula (I) has the formula Q-CH2 CH(CH2 (C6H5))C(O)OH, Q-CH2 CH2 CH(C6H5)C(O)NHCH2 C(O)OCH2 CH2, Q-CH2 CH2 CH(C6H5)C(O)NHCH2 C(O)OH, Q-CH2 CH2 CH(C6H5)C(O)NHCH2 C(O)NHCH3, Q-CH2 CH2 CH(C6H5)C(O)NHCH2 C(O)NHCH2 CH3, G-NH(CH2)2 C(O)NH2, G-NH(CH2)2 C(O)NHCH3, G-NHCH2 C(O)NH2, G-NHCH2 C(O)NHCH3, G-NHCH3 C(O)NHCH2 CH3, G-NH(CH2)3 C(O)OCH2 CH3, G-NH(CH2)3 C(O)NHCH3, G-NH(CH2)2 C(O)OH, G-NH(CH2)3 C(O)OH, Q-CH2 CH(CH2 (C6 H11))C(O)NHCH2 C(O)OH, Q-CH2 CH(CH2 (C6 H11))C(O)NH(CH2)2 C(O)OH, Q-CH2 CH(CH2 (C6H11))C(O)NH(CH2)2 C(O)NH2, Z-NHCH2 C(O)OCH2 CH3, Z-NHCH2 C(O)OH, Z-NHCH2 C(O)NH2, Z-NHCH2 C(O)N(CH3)2, Z-NHCH2 C(O)NHCH(CH3)2, Z-NHCH2 C(O)OCH2 CH(CH3)2, Z-NH(CH2)2 C(O)OCH2 (C6H5), Z-NH(CH2 C(O)OH, Z-NH(CH2)2 C(O)NHCH2 CH3, Z-NH(CH2)3 C(O)NHCH3, Z-NHCH2 C(O)NHCH2 C(O)OH, Z-NHCH2 C(O)OCH2 C(O)OCH3, Z-NHCH2 C(O)O(CH2)4 CH3, Z-NHCH2 C(O)OCH2 C(O)NHCH3, Z-NHCH2 C(O)O-(4-methoxycyclohexyl), Z-NHCH2 C(O)OCH2 C(O)NHCH2 (C6H5) or Z-NHCH2 C(O)OCH(CH3)OC(O)CH3; wherein Q, G and Z are as defined above.

In some embodiments, the compound of formula (I) has the formula (3R,4R,S)-Z-NHCH2 C(O)OCH2 CH(CH3)2, (+)-Z-NHCH2 C(O)OH, (−)-Z-NHCH2 C(O)OH, (3R,4R,R)-Z-NHCH2 C(O)—OCH2 CH(CH3)2, (3S,4S,S)-Z-NHCH2 C(O)OCH2 CH(CH3)2, (3S,4S,R)-Z-NHCH2 C(O)OCH2 CH(CH3)2, (3R,4R)-Z-NHCH2 C(O)NHCH2 (C6H5) or (3R,4R)-G-NH(CH2)3 C(O)OH, where Z and G are as defined above. In some embodiments, the compound of formula (I) has the formula (+)-Z-NHCH2 C(O)OH or (−)-Z-NHCH2 C(O)OH where Z is as defined above.

Compounds of formula (I) that act locally, such as on the gut, have high potency and are orally active. An embodiment of the present invention is the compound (+)-Z-NHCH2 C(O)OH, i.e., the compound of the following formula (II).

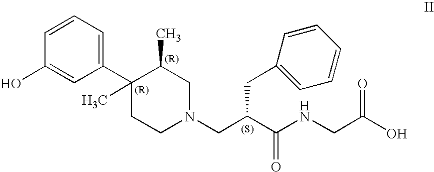

The compound of formula (II) has low solubility in water except at low or high pH conditions. Zwitterionic character may be inherent to the compound, and may impart desirable properties such as poor systemic absorption and sustained local affect on the gut following oral administration.

In an alternate embodiment, the methods of the present invention may involve administering to a patient a peripheral mu-opioid receptor antagonist that is a quaternary morphinan compound. Examples of quaternary morphinan compounds that may be suitable for use in the methods of the present invention include, for example, quaternary salts of N-methylnaltrexone, N-methylnaloxone, N-methylnalorphine, N-diallylnormorphine, N-allyllevallorphan and N-methylnalmefene.

In yet another alternate embodiment, the methods of the present invention may involve administering to a patient a peripheral mu-opioid receptor antagonist in the form of an opium alkaloid derivative. The term “opium alkaloid derivative”, as used herein, refers to peripheral mu-opioid receptor antagonists that are synthetic or semi-synthetic derivatives or analogs of opium alkaloids. In preferred form, the opium alkaloid derivatives employed in the methods of the present invention exhibit high levels of morphine antagonism, while exhibiting reduced, and preferably substantially no, agonist activity. The term “substantially no agonist activity”, as used herein in connection with the opium alkaloid derivatives, means that the maximal response with respect to electrically stimulated guinea pig ileum, at a concentration of 1 μM, is about 60% or less relative to morphine. In some embodiments, the opium alkaloid derivatives employed in the present methods have a maximal response with respect to guinea pig ileum, at a concentration of 1 μM, of about 50% or less relative to morphine, with a maximal response of about 40% or less being more preferred. In some embodiments, the opium alkaloid derivatives employed in the present methods have a maximal response with respect to guinea pig ileum, at a concentration of 1 μM, of about 30% or less relative to morphine, with a maximal response of about 20% or less. In still other embodiments, the opium alkaloid derivatives employed in the present methods have a maximal response with respect to guinea pig ileum, at a concentration of 1 μM, of about 10% or less relative to morphine. In certain embodiments, the opium alkaloid derivatives have a maximal response with respect to guinea pig ileum, at a concentration of 1 μM, of about 0% (i.e., no response).

Suitable methods for determining maximal response of opium alkaloid derivatives with respect to electrically stimulated guinea pig ileum are described, for example, in U.S. Pat. Nos. 4,730,048 and 4,806,556, the disclosures of which are hereby incorporated herein by reference, in their entireties.

In some embodiments, the opium alkaloid derivatives employed in the methods of the present invention have the following formulas (III) or (IV):

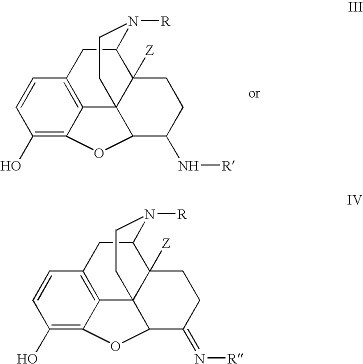

wherein:
R is alkyl, cycloalkyl-substituted alkyl, aryl, aryl-substituted alkyl or alkenyl;
Z is hydrogen or OH;
R′ is X′-J(L)(T), wherein:
J is alkylene or alkenylene;
L is hydrogen, amino, or alkyl optionally substituted with CO2H, OH or phenyl; and
T is CO2H, SO3H, amino or guanidino;
X′ is a direct bond or C(═O); and
R″ is NH-J(L)(T) or guanidino; or a stereoisomer, prodrug, or pharmaceutically acceptable salt, hydrate or N-oxide thereof.

In the compounds of formulas (III) and (IV) above, R is alkyl, cycloalkyl-substituted alkyl, aryl, aryl-substituted alkyl or alkenyl. In some embodiments, R is C1-C5 alkyl, C3-C6 cycloakyl-substituted alkyl, aryl, arylalkyl or trans-C2-C5 alkenyl. In some embodiments, R is C1-C3 alkyl, allyl or cyclopropylmethyl, with cyclopropylmethyl being even more preferred.

In the compounds of formulas (III) and (IV) above, Z is hydrogen or OH. In some embodiments, Z is OH.

In the compounds of formulas (III) and (IV), R′ is X-J(L)(T) and R″ is NH-J(L)(T) or guanidino.

In the definitions of R′ and R″, G is alkylene or alkenylene. In some embodiments, J is C1-C5 alkylene, C2-C6 alkylene interrupted by an oxygen atom, or C2-C5 alkenylene. In the definitions of R′ and R″, L is hydrogen, amino, or alkyl optionally substituted with CO2H, OH or phenyl. In some embodiments, L is hydrogen, amino, or C1-C5 alkyl optionally substituted with CO2H, OH or phenyl. In some embodiments, L is hydrogen or amino.

In the definitions of R′ and R″, T is CO2H, SO3H, amino or guanidino. In some embodiments, T is CO2H or guanidino.

In the definition of R′, X is a direct bond or C(═O).

Important opioid alkaloid derivatives that may be employed in the methods of the present invention include compounds of formula (III) wherein R is cyclopropylmethyl, Z is OH, and R′ is selected from C(═O)(CH2)2 CO2H, C(═O)(CH2)3 CO2H, C(═O)CH—CHCO2H, C(═O)CH2 OCH2 CO2H, C(═O)CH(NH2)(CH2)3 NHC(—NH)NH2 or C(═O)CH(NH2)CH2 CO2H. Also important are opioid alkaloid derivatives of formula (III) wherein R is cyclopropylmethyl, Z is OH, and R′ is CH2CO2H. In other embodiments, the opioid alkaloid derivatives that may be employed in the methods of the present invention include compounds of formula (IV) wherein R is cyclopropylmethyl, Z is OH, and R″ is NHCH2CO2H. For example, N-methylnaltrexone (or methylnaltrexone, MNTX) has the following formula (V):

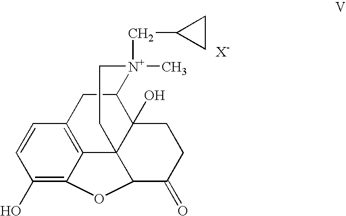

Methods for synthesis, formulating and manufacturing MNTX have been described in a co-pending U.S. patent application Ser. No. (number not yet assigned) titled “SYNTHESIS OF (R)-N-METHYLNALTREXONE”, Attorney Docket No. PO453.70119US01, filed on May 25, 2006, and hereby incorporated by reference in its entirety.

Other opioid alkaloid derivatives that may be employed in the methods of the present invention are described, for example, in U.S. Pat. Nos. 4,730,048 and 4,806,556, the disclosures of which are hereby incorporated herein by reference, in their entireties.

In still other embodiments, the methods of the present invention may involve administering to a patient a peripheral mu-opioid receptor antagonist compound in the form of a quaternary benzomorphan compound. In some embodiments, the quaternary benzomorphan compounds employed in the methods of the present invention exhibit high levels of morphine antagonism, while exhibiting reduced, and preferably substantially no, agonist activity. The term “substantially no agonist activity”, as used herein in connection with the quaternary benzomorphan compounds, means that the maximal response with respect to electrically stimulated guinea pig ileum, at a concentration of 1 μM, is about 60% or less relative to morphine. In some embodiments, the quaternary benzomorphan compounds employed in the present methods have a maximal response with respect to guinea pig ileum, at a concentration of 1 μM, of about 50% or less relative to morphine, with a maximal response of about 40% or less being more preferred. In some embodiments, the quaternary benzomorphan compounds employed in the present methods have a maximal response with respect to guinea pig ileum, at a concentration of 1 μM, of about 30% or less relative to morphine, with a maximal response of about 20% or less being. In some embodiments, the quaternary benzomorphan compounds employed in the present methods have a maximal response with respect to guinea pig ileum, at a concentration of 1 μM, of about 10% or less relative to morphine. In certain embodiments, the quaternary benzomorphan compounds have a maximal response with respect to guinea pig ileum, at a concentration of 1 μM, of about 0% (i.e., no response).

In some embodiments, the quaternary benzomorphan compounds employed in the methods of the present invention have the following formula (VI):

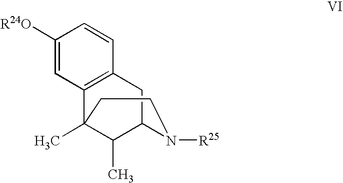

where:
R24 is hydrogen or acyl; and
R25 is alkyl or alkenyl; or a stereoisomer, prodrug, or pharmaceutically acceptable salt, hydrate or N-oxide thereof.

In the above formula (VI), R24 is hydrogen or acyl. In some embodiments, R24 is hydrogen or C1-C6 acyl. In some embodiments, R24 is hydrogen or C1-C2 acyl. In some embodiments, R24 is hydrogen or acetoxy, with hydrogen being still more preferred.

In the above formula (VI), R25 is alkyl or alkenyl. In some embodiments, R25 is C1-C6 alkyl or C2-C6 alkenyl. In some embodiments, R5 is C1-C3 alkyl or C2-C3 alkenyl. In some embodiments, R25 is propyl or allyl.

Important quaternary benzomorphan compounds that may be employed in the methods of the present invention include the following compounds of formula (VI): 2′-hydroxy-5,9-dimethyl-2,2-diallyl-6,7-benzomorphanium-bromide; 2′-hydroxy-5,9-dimethyl-2-n-propyl-6,7-benzomorphan; 2′-hydroxy-5,9-dimethyl-2-allyl-6,7-benzomorphan; 2′-hydroxy-5,9-dimethyl-2-n-propyl-2-allyl-6,7-benzomorphanium-bromide; 2′-hydroxy-5,9-dimethyl-2-n-propyl-2-propargyl-6,7-benzomorphanium-bromide; and 2′-acetoxy-5,9-dimethyl-2-n-propyl-2-allyl-6,7-benzomorphanium-bromide.

Other quaternary benzomorphan compounds that may be employed in the methods of the present invention are described, for example, in U.S. Pat. No. 3,723,440, the disclosures of which are hereby incorporated herein by reference, in their entirety.

Other mu opioid receptor antagonists which may be employed in the methods and compositions of the present invention, in addition to those exemplified above, would be readily apparent to one of ordinary skill in the art, once armed with the teachings of the present disclosure.

The compounds employed in the methods of the present invention may exist in prodrug form. As used herein, “prodrug” is intended to include any covalently bonded carriers which release the active parent drug, for example, as according to formulas (I) or (II) or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example formula (I), may be prepared by modifying functional groups present in the compound in such a way that the modifications are cleaved, either in routine manipulation or in vivo, to yield the pharmacologically active moiety.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

The compounds employed in the methods of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

Compounds employed in the present methods may contain one, or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups include the benzyloxycarbonyl group and the tert-butyloxycarbonyl group. Other protecting groups that may be employed in accordance with the present invention may be described in Greene, T. W. and Wuts, P. G. M., Protective Groups in Organic Synthesis 2d. Ed., Wiley & Sons, 1991.

Piperidine-N-alkylcarboxylate compounds according to the present invention may be synthesized employing methods taught, for example, in U.S. Pat. Nos. 5,250,542, 5,434,171, 5,159,081, and 5,270,328, the disclosures of which are hereby incorporated herein by reference in their entireties. For example, the 3-substituted-4-methyl-4-(3-hydroxy- or alkanoyloxyphenyl)piperidine derivatives employed as starting materials in the synthesis of the present compounds may be prepared by the general procedure taught in U.S. Pat. No. 4,115,400 and U.S. Pat. No. 4,891,379, the disclosures of which are hereby incorporated herein by reference in their entireties. The starting material for the synthesis of compounds described herein, (3R,4R)-4-(3-hydroxyphenyl)-3,4-dimethylpiperidine, may be prepared by the procedures described in U.S. Pat. No. 4,581,456, the disclosures of which are hereby incorporated herein by reference, in their entirety, but adjusted as described such that the .beta.-stereochemistry is preferred.

The first step of the process may involves the formation of the 3-alkoxyphenyllithium reagent by reacting 3-alkoxybromobenzene with an alkyllithium reagent. This reaction may be performed under inert conditions and in the presence of a suitable non-reactive solvent such as dry diethyl ether or preferably dry tetrahydrofuran. Preferred alkyllithium reagents used in this process are n-butyllithium, and especially sec-butyllithium. Generally, approximately an equimolar to slight excess of alkyllithium reagent may be added to the reaction mixture. The reaction may be conducted at a temperature of from about −20° C. and about −100° C., more preferably from about −50° C. to about −55° C.

Once the 3-alkoxyphenyllithium reagent has formed, approximately an equimolar quantity of a 1-alkyl-4-piperidone may be added to the mixture while maintaining the temperature between −20° C. and −100° C. The reaction is typically complete after about 1 to 24 hours. At this point, the reaction mixture may be allowed to gradually warm to room temperature. The product may be isolated by the addition to the reaction mixture of a saturated sodium chloride solution to quench any residual lithium reagent. The organic layer may be separated and further purified if desired to provide the appropriate 1-alkyl-4-(3-alkoxyphenyl)piperidinol derivative.

The dehydration of the 4-phenylpiperidinol prepared above may be accomplished with a strong acid according to well known procedures. While dehydration occurs in various amounts with any one of several strong acids such as hydrochloric acid, hydrobromic acid, and the like, dehydration is preferably conducted with phosphoric acid, or especially p-toluenesulfonic acid in toluene or benzene. This reaction may be typically conducted under reflux conditions, more generally from about 50° C. and 150° C. The product thus formed may be isolated by basifying an acidic aqueous solution of the salt form of the product and extracting the aqueous solution with a suitable water immiscible solvent. The resulting residue following evaporation can then be further purified if desired.

The 1-alkyl-4-methyl-4-(3-alkoxyphenyl)tetrahydropyridine derivatives may be prepared by a metalloenamine alkylation. This reaction is preferably conducted with n-butyllithium in tetrahydrofuran (THF) under an inert atmosphere, such as nitrogen or argon. Generally, a slight excess of n-butyllithium may be added to a stirring solution of the 1-alkyl-4-(3-alkoxyphenyl)-tetrahydropyridine in THF cooled to a temperature in the range of from about is −50° C. to about 0° C., more preferably from about −20° C. to −10° C. This mixture may be stirred for approximately 10 to 30 minutes followed by the addition of approximately from 1.0 to 1.5 equivalents of methyl halide to the solution while maintaining the temperature of the reaction mixture below 0° C. After about 5 to 60 minutes, water may be added to the reaction mixture and the organic phase may be collected. The product can be purified according to standard procedures, but the crude product is preferably purified by either distilling it under vacuum or slurrying it in a mixture of hexane:ethyl acetate (65:35, v:v) and silica gel for about two hours. According to the latter procedure, the product may be then isolated by filtration followed by evaporating the filtrate under reduced pressure.

The next step in the process may involve the application of the Mannich reaction of aminomethylation to non-conjugated, endocyclic enamines. This reaction is preferably carried out by combining from about 1.2 to 2.0 equivalents of aqueous formaldehyde and about 1.3 to 2.0 equivalents of a suitable secondary amine in a suitable solvent. While water may be the preferred solvent, other non-nucleophilic solvents, such as acetone and acetonitrile can also be employed in this reaction. The pH of this solution may be adjusted to approximately 3.0 to 4.0 with an acid that provides a non-nucleophilic anion. Examples of such acids include sulfuric acid, the sulfonic acids such as methanesulfonic acid and p-toluenesulfonic acid, phosphoric acid, and tetrafluoroboric acid, with sulfuric acid being preferred. To this solution may be added one equivalent of a 1-alkyl-4-methyl-4-(3-alkoxyphenyl)tetrahydropyridine, typically dissolved in aqueous sulfuric acid, and the pH of the solution may be readjusted with the non-nucleophilic acid or a suitable secondary amine. The pH is preferably maintained in the range of from about 1.0 to 5.0, with a pH of about 3.0 to 3.5 being more preferred during the reaction. The reaction is substantially complete after about 1 to 4 hours, more typically about 2 hours, when conducted at a temperature in the range of from about 50° C. to about 80° C., more preferably about 70° C. The reaction may then be cooled to approximately 30° C., and added to a sodium hydroxide solution. This solution may then be extracted with a water immiscible organic solvent, such as hexane or ethyl acetate, and the organic phase, following thorough washing with water to remove any residual formaldehyde, may be evaporated to dryness under reduced pressure.

The next step of the process may involve the catalytic hydrogenation of the prepared 1-alkyl-4-methyl-4-(3-alkoxyphenyl)-3-tetralydropyridinemethanamine to the corresponding trans-1-alkyl-3,4-dimethyl-4-(3-alkoxyphenyl)piperidine. This reaction actually occurs in two steps. The first step is the hydrogenolysis reaction wherein the exo C—N bond is reductively cleaved to generate the 3-methyltetrahydropyridine. In the second step, the 2,3-double bond in the tetrahydropyridine ring is reduced to afford the desired piperidine ring.

Reduction of the enamine double bond introduced the crucial relative stereochemistry at the 3 and 4 carbon atoms of the piperidine ring. The reduction generally does not occur with complete stereoselectivity. The catalysts employed in the process may be chosen from among the various palladium and preferably platinum catalysts.

The catalytic hydrogenation step of the process is preferably conducted in an acidic reaction medium. Suitable solvents for use in the process include the alcohols, such as methanol or ethanol, as well as ethyl acetate, tetrahydrofuran, toluene, hexane, and the like.

Proper stereochemical outcome may be dependent on the quantity of catalyst employed. The quantity of catalyst required to produce the desired stereochemical result may be dependent upon the purity of the starting materials in regard to the presence or absence of various catalyst poisons.

The hydrogen pressure in the reaction vessel may not be critical but can be in the range of from about 5 to 200 psi. Concentration of the starting material by volume is preferably around 20 mL of liquid per gram of starting material, although an increased or decreased concentration of the starting material can also be employed. Under the conditions specified herein, the length of time for the catalytic hydrogenation may not be critical because of the inability for over-reduction of the molecule. While the reaction can continue for up to 24 hours or longer, it may not be necessary to continue the reduction conditions after the uptake of the theoretical two moles of hydrogen. The product may then be isolated by filtering the reaction mixture for example through infusorial earth, and evaporating the filtrate to dryness under reduced pressure. Further purification of the product thus isolated may not be necessary and preferably the diastereomeric mixture may be carried directly on to the following reaction.

The alkyl substituent may be removed from the 1-position of the piperidine ring by standard dealkylation procedures. Preferably, a chloroformate derivative, especially the vinyl or phenyl derivatives, may be employed and removed with acid. Next, the prepared alkoxy compound may be dealkylated to the corresponding phenol. This reaction may be generally carried out by reacting the compound in a 48% aqueous hydrobromic acid solution. This reaction may be substantially complete after about 30 minutes to 24 hours when conducted at a temperature of from about 50° C. to about 150° C., more preferably at the reflux temperature of the reaction mixture. The mixture may then be worked up by cooling the solution, followed by neutralization with base to an approximate pH of 8. This aqueous solution may be extracted with a water immiscible organic solvent. The residue following evaporation of the organic phase may then be used directly in the following step.

The compounds employed as starting materials to the compounds of the invention can also be prepared by brominating the 1-alkyl-4-methyl-4-(3-alkoxyphenyl)-3-tetrahydropyridinemethanamine at the 3-position, lithiating the bromo compound thus prepared, and reacting the lithiated intermediate with a methylhalide, such as methyl bromide to provide the corresponding 1-alkyl-3,4-dimethyl-4-(3-alkoxyphenyl)tetrahydropyridinemethanamine. This compound may then be reduced and converted to the starting material as indicated above.

The compounds of the present invention can exist as the individual stereoisomers. Preferably reaction conditions are adjusted as disclosed in U.S. Pat. No. 4,581,456 or as set forth in Example 1 of U.S. Pat. No. 5,250,542 to be substantially stereoselective and provide a racemic mixture of essentially two enantiomers. These enantiomers may then be resolved. A procedure which may be employed to prepare the resolved starting materials used in the synthesis of these compounds includes treating a racemic mixture of alkyl-3,4-dimethyl-4-(3-alkoxyphenyl)piperidine with either (+)- or ditoluoyl tartaric acid to provide the resolved intermediate. This compound may then be dealkylated at the 1-position with vinyl chloroformate and finally converted to the desired 4-(3-hydroxyphenyl)piperidine isomer.

As will be understood by those skilled in the art, the individual enantiomers of the invention can also be isolated with either (+) or (−) dibenzoyl tartaric acid, as desired, from the corresponding racemic mixture of the compounds of the invention. Preferably the (+)-trans enantiomer is obtained.

Although the (+)trans-3,4 stereoisomer is preferred, all of the possible stereoisomers of the compounds described herein are within the contemplated scope of the present invention. Racemic mixtures of the stereoisomers as well as the substantially pure stereoisomers are within the scope of the invention. The term “substantially pure”, as used herein, refers to at least about 90 mole percent, more preferably at least about 95 mole percent and most preferably at least about 98 mole percent of the desired stereoisomer is present relative to other possible stereoisomers.

Intermediates can be prepared by reacting a 3,4-alkyl-substituted-4-(3-hydroxyphenyl)piperidine with a compound of the formula LCH2 (CH2), C1-CHR3C(O)E where L is a leaving group such as chlorine, bromine or iodine, E is a carboxylic acid, ester or amide, and R3 and n are as defined hereinabove. Preferably L may be chlorine and the reaction is carried out in the presence of a base to alkylate the piperidine nitrogen. For example 4-chloro-2-cyclohexylbutanoic acid, ethyl ester can be contacted with (3R,4R)-4-(3-hydroxyphenyl)-3,4-dimethylpiperidine to provide 4-[(3R,4R)-4-(3-hydroxyphenyl)-3,4-dimethyl-1-piperidine]butanoic acid, ethyl ester. Although the ester of the carboxylic acid may be preferred, the free acid itself or an amide of the carboxylic acid may be used.

In alternative synthesis, the substituted piperidine can be contacted with a methylene alkyl ester to alkylate the piperidine nitrogen. For example, 2-methylene-3-phenylpropanoic acid, ethyl ester can be contacted with a desired piperidine to provide 2-benzyl-3-piperidinepropanoic acid ethyl ester.

Another synthetic route can involve the reaction of a substituted piperidine with a haloalkylnitrile. The nitrile group of the resulting piperidine alkylnitrile can be hydrolyzed to the corresponding carboxylic acid.

With each of the synthetic routes, the resulting ester or carboxylic acid can be reacted with an amine or alcohol to provide modified chemical structures. In the preparation of amides, the piperidine-carboxylic acid or -carboxylic acid ester may be reacted with an amine in the presence of a coupling agent such as dicyclohexylcarbodiimide, boric acid, borane-trimethylamine, and the like. Esters can be prepared by contacting the piperidine-carboxylic acid with the appropriate alcohol in the presence of a coupling agent such as p-toluenesulfonic acid, boron trifluoride etherate or N,N′-carbonyldiimidazole. Alternatively, the piperidine-carboxylic acid chloride can be prepared using a reagent such as thionyl chloride, phosphorus trichloride, phosphorus pentachloride and the like. This acyl chloride can be reacted with the appropriate amine or alcohol to provide the corresponding amide or ester.

Opium alkaloid derivatives according to the present invention may be synthesized employing methods taught, for example, in U.S. Pat. Nos. 4,730,048 and 4,806,556, the disclosures of which are hereby incorporated herein by reference in their entireties. For example, opium alkaloid derivatives of formula (III) may be prepared by attaching hydrophilic, ionizable moieties R′ and R″ to the 6-amino group of naltrexamine (formula (III) where R is (cyclopropyl)methyl, Z is OH and R! is H) or oxymorphamine (formula (III) where R is CH3, Z is OH and R! is H). The opium alkaloid derivatives of formula IV may be prepared by converting the 6-keto-group of oxymorphone (formula (VII) where R is CH3 and Z is OH) or naltrexone (formula (VII) where R is (cyclopropyl)methyl and Z is OH; see also formula V) to the ionizable, hydrophilic group (R″N═) by a Schiff base reaction with a suitable amino-compound.

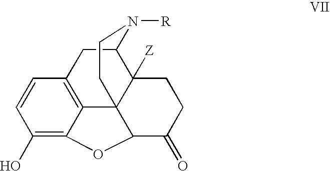

In a similar fashion, deoxy-opiates of formulae (III) and (IV) wherein Z is hydrogen may be prepared from readily available starting materials.

The compounds of formula (VII) may be synthesized employing methods taught, for example, in U.S. Pat. No. 3,723,440, the disclosures of which are hereby incorporated herein by reference in their entirety.

The antagonist may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the antagonist may be incorporated with excipient and used in the form of ingestible tablets, buccal tablets, troches, capsules, elixirs, suspensions, syrups, wafers, and the like. The amount of antagonist in such therapeutically useful compositions is adjusted to achieve suitable dosages using routine techniques within the skill in the art. An exemplary dosage for an antagonist is an oral dosage unit form containing from about 0.1 to about 1000 mg of antagonist.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose, saccharin, and/or a flavoring agent, such as peppermint, oil of wintergreen or cherry flavoring. When the unit dosage form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye, and/or flavoring, such as cherry or orange flavor. Of course, any material used in preparing any unit dosage form is preferably pharmaceutically pure and substantially non-toxic in the amount employed. In addition, the active compound may be incorporated into sustained-release preparations and formulations.

The antagonist may also be administered parenterally or intraperitoneally. Solutions of the antagonists in unmodified form or as pharmacologically acceptable salts are contemplated and can be prepared in water suitably mixed with a surfactant, such as hydroxypropylcellulose. A dispersion can also be prepared in glycerol, liquid polyethylene glycols, preferably a high molecular weight polyethylene glycol of average molecular weight at least 15 kDa, mixtures thereof and in oils. In addition, any route of administration disclosed herein or known in the art may be used.

Pharmacologically and pharmaceutically acceptable salts for inclusion in administrable compositions include, but are not limited to, those prepared from the following acids: hydrochloric, hydrobromic, sulfuric, nitric, phosphoric, maleic, acetic, salicylic, p-toluenesulfonic, tartaric, citric, methanesulfonic, formic, succinic, naphthalene-2-sulfonic, palmoic, 3-hydroxy-2-naphthalenecarboxylic, and benzene sulfonic. Suitable buffering agents include, but are not limited to, acetic acid and salts thereof (1-2% WN); citric acid and salts thereof (1-3% WN); boric acid and salts thereof (0.5-2.5% WN); and phosphoric acid and salts thereof (0.8-2% WN). Suitable preservatives include, but are not limited to, benzalkonium chloride (0.003-0.03% WN); 5 chlorobutanol (0.3-0.9% WIN); parabens (0.01-0.25% WN) and thimerosal (0.004-0.02% WN). For ease of administration, a pharmaceutical composition of the peripheral opioid antagonist may also contain one or more pharmaceutically acceptable excipients, such as lubricants, diluents, binders, carriers, and disintegrants. Other auxiliary agents may include, e.g., stabilizers, wetting agents, emulsifiers, salts for influencing osmotic pressure, coloring, flavoring and/or aromatic active compounds.

A pharmaceutically acceptable carrier or excipient refers to a non-toxic solid, semi-solid or liquid filler, diluent, encapsulating material or formulation auxiliary of any type. For example, suitable pharmaceutically acceptable carriers, diluents, solvents or vehicles include, but are not limited to, water, salt (buffer) solutions, alcohols, gum arabic, mineral and vegetable oils, benzyl alcohols, polyethylene glycols, gelatin, carbohydrates such as lactose, amylose or starch, magnesium stearate, talc, silicic acid, viscous paraffin, vegetable oils, fatty acid monoglycerides and diglycerides, pentaerythritol fatty acid esters, hydroxy methylcellulose, polyvinyl pyrrolidone, and the like. Proper fluidity may be maintained, for example, by the use of coating materials such as lecithin, by the maintenance of the required particle size in the case of dispersions and by the use of surfactants. Prevention of the action of microorganism may be ensured by the inclusion of various antibacterial and antifungal agents such as paraben, chlorobutanol, phenol, sorbic acid and the like.

If a pharmaceutically acceptable solid carrier is used, the dosage form of the antagonist(s) may be tablets, capsules, powders, suppositories, or lozenges. If a liquid carrier is used, soft gelatin capsules, transdermal patches, aerosol sprays, topical cream, syrups or liquid suspensions, emulsions or solutions may be the dosage form.

For parental application, particularly suitable are injectable, sterile solutions, preferably non-aqueous or aqueous solutions, as well as dispersions, suspensions, emulsions, or implants, including suppositories. Ampoules are convenient forms in which to administer unit dosages.

The pharmaceutical forms suitable for injectable use include, for example, sterile aqueous solutions or dispersions and sterile powders for the extemporaneous preparation of sterile injectable solutions or dispersions. In all cases, the form is preferably sterile; for administration via injection, the form is preferably sufficiently non-viscous to provide acceptable syringeability according to norms established in the art. The antagonist forms are preferably stable under the conditions of manufacture and storage and are preferably resistant to untoward contamination. The carrier may be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating, such as lecithin, by the maintenance of the required particle size in the case of a dispersion, and by the use of surfactants. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients disclosed above, as required, followed by filter sterilization or sterilization via irradiation. Generally, dispersions may be prepared by incorporating the sterilized active ingredient into a sterile vehicle which contains the basic dispersion medium and the required other ingredients from those disclosed above. In the case of sterile powders for the preparation of sterile injectable solutions, the preferred methods of preparation may include vacuum drying and/or a freeze drying technique which yields a powder of the active ingredient, plus any additional desired ingredient from the previously sterilized solution thereof.

An injectable depot form may also be suitable and may be made by forming a microcapsule matrix of the drug in a biodegradable polymer such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use.

For enteral application, particularly suitable are tablets, dragees, liquids, drops, suppositories, or capsules such as soft gelatin capsules. A syrup, elixir, or the like can be used wherein a sweetened vehicle is employed.

Another delivery system may include a time-release, delayed-release or sustained-release (extended release) delivery system. Such a system can avoid repeated administrations of a compound of the invention, increasing convenience to the patient and the physician and maintaining sustained plasma levels of compounds where desired. Many types of controlled-release delivery systems are available and known to those of ordinary skill in the art. Sustained- or controlled-release compositions can be formulated, e.g., as liposomes or by protecting the active compound with differentially degradable coatings, such as by microencapsulation, multiple coatings, and the like.

For example, compounds of the invention may be combined with pharmaceutically acceptable sustained-release matrices, such as biodegradable polymers, to form therapeutic compositions. A sustained-release matrix, as used herein, is a matrix typically composed of one or more polymers that are degradable by enzymatic or acid-base hydrolysis or by dissolution. Once inserted into the body, the matrix is acted upon by enzymes and body fluids. A sustained-release matrix may be desirably chosen from biocompatible materials such as liposomes, polymer-based systems such as polylactides (polylactic acid), polyglycolide (polymer of glycolic acid), polylactide co-glycolide (copolymers of lactic acid and glycolic acid), polyanhydrides, poly(ortho)esters, polysaccharides, polyamino acids, hyaluronic acid, collagen, chondroitin sulfate, polynucleotides, polyvinyl propylene, polyvinylpyrrolidone, and silicone; nonpolymer systems are composed of chemical components such as carboxylic acids, fatty acids, phospholipids, amino acids, lipids such as sterols, hydrogel release systems, silastic systems, peptide-based systems, implants, and the like. Specific examples include, but are not limited to: (a) erosional systems in which the polysaccharide is contained in a form within a matrix, as disclosed in U.S. Pat. Nos. 4,452,775, 4,675,189, and 5,736,152 (herein incorporated by reference in their entireties), and (b) diffusional systems in which an active component permeates, at a controlled rate, from a polymer such as described in U.S. Pat. Nos. 3,854,480, 5,133,974 and 5,407,686 (herein incorporated by reference in their entireties). In addition, pump-based hard-wired delivery systems can be used, some of which are adapted for implantation. Suitable enteric coatings are described in PCT publication No. WO 98125613 and U.S. Pat. No. 6,274,591, both incorporated herein by reference.

Use of a long-term sustained-release implant may be particularly suitable for treatment of chronic conditions. “Long-term” release, as used herein, means that the implant is constructed and arranged to deliver therapeutic levels of the active ingredient for at least 7 days, and suitably 30 to 60 days. Long-term sustained-release implants are well-known to those of ordinary skill in the art and include some of the release system described above.

For topical application, one embodiment employs, as a nonsprayable form, a viscous to semi-solid or solid form comprising a carrier compatible with topical application and having a dynamic viscosity preferably greater than water. Suitable formulations include, but are not limited to, solutions, suspensions, emulsions, cream, ointments, powders, liniments, salves, aerosols, and the like, which are optionally sterilized or mixed with auxiliary agents, e.g., preservatives, and the like.

Transdermal or iontophoretic delivery of pharmaceutical compositions of the peripheral opioid antagonists is also contemplated.

The therapeutic compounds of this invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds of the present invention that will be most suitable for prophylaxis or treatment will vary with the form of administration, the particular antagonist chosen, and the physiological characteristics of the particular patient under treatment. Typically, a daily dosage may range from about 0.001 to about 100 milligrams of the peripheral μ-opioid receptor antagonist (and all combinations and subcombinations of ranges therein), per kilogram of patient body weight. Preferably, the a daily dosage may be about 0.01 to about 10 milligrams of the peripheral μ-opioid receptor antagonist per kilogram of patient body weight. Also preferred is a daily dosage of about 0.1 milligrams of the peripheral μ-opioid receptor antagonist per kilogram of patient body weight. With regard to a typical dosage form, for example in tablet form, the peripheral μ-opioid receptor antagonist is present in an amount of about 0.1 to about 4 milligrams.

In one embodiment of this invention the product is orally administered wherein an antagonist is enteric coated. By enteric coating an antagonist, it is possible to control its release into the gastrointestinal tract such that the antagonist is not released in the stomach, but rather is released in the intestine. Another embodiment of this invention where oral administration is desired provides for a combination product wherein one of the products, e.g., a μ-opioid receptor antagonist, is coated with a sustained-release material which effects a sustained-release throughout the gastrointestinal tract and also serves to minimize physical contact between the μ-opioid receptor antagonist and any other compound in the product. Furthermore, the sustained-released component can be additionally enteric coated such that the release of this component occurs only in the intestine. Still another approach involves the formulation of a combination product in which the one component is coated with a sustained and/or enteric release polymer, and the other component is also coated with a polymer such as a low-viscosity grade of hydroxypropyl methylcellulose (HPMC) or other appropriate material as known in the art, in order to further separate the active components. The polymer coating serves to form an additional barrier to interaction with the other component.

In some embodiments, compounds of the invention are administered in a dosing regimen that provides a continuous dose of the compound to a subject, i.e., a regimen that eliminates the variation in internal drug levels found with conventional regimens. Suitably, a continuous dose may be achieved by administering the compound to a subject on a daily basis using any of the delivery methods disclosed herein. In one embodiment, the continuous dose may be achieved using continuous infusion to the subject, or via a mechanism that facilitates the release of the compound over time, for example, a transdermal patch, or a sustained release formulation. Suitably, compounds of the invention are continuously released to the subject in amounts sufficient to maintain a concentration of the compound in the plasma of the subject effective to inhibit or reduce cell barrier dysfunction. Compounds in accordance with the invention, whether provided alone or in combination with other therapeutic agents, are provided in an effective amount to prevent, reduce or eliminate a cell barrier dysfunction. It will be understood, however, that the total daily usage of the compounds and compositions of the present invention will be decided by the attending physician within the scope of sound medical judgment. The specific effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the level of ordinary skill in the art to start doses of the compound at levels lower than those required to achieve the desired therapeutic effect and to gradually increase the dosage until the desired effect is achieved.

If desired, the effective daily dose may be divided into multiple doses for purposes of administration. Consequently, single-dose compositions may contain such amounts or submultiples thereof to make up the daily dose. As noted, those of ordinary skill in the art will readily optimize effective doses and co-administration regimens as determined by good medical practice and the clinical condition of the individual patient.

Generally, oral doses of the opioid receptor antagonists, particularly peripheral receptor antagonists, will range from about 1 to about 80 mg/kg body weight per day. It is expected that oral doses in the range from 2 to 20 mg/kg body weight will yield beneficial results. Generally, parenteral administration, including intravenous and subcutaneous administration, will range from about 0.001 to 5 mg/kg body weight. It is expected that doses ranging from 0.05 to 0.5 mg/kg body weight will yield the desired results. Dosage may be adjusted appropriately to achieve desired drug levels, local or systemic, depending on the mode of administration. For example, it is expected that the dosage for oral administration of the opioid antagonists in an enterically-coated formulation would be from 10 to 30% of the non-coated oral dose. In the event that the response in a patient is insufficient to such doses, even higher doses (or effectively higher dosages by a different, more localized, delivery route) may be employed to the extent that patient tolerance permits. Multiple doses per day are contemplated to achieve appropriate systemic levels of compounds. Appropriate system levels can be determined by, for example, measurement of the patient's plasma level for the drug using routine HPLC methods known to those of skill in the art.

In some embodiments of the invention, the opioid receptor antagonists are co-administered with an opioid compound. The term “co-administration” is meant to refer to a combination therapy by any administration route in which two or more agents are administered to a patient or subject. Co-administration of agents may also be referred to as combination therapy or combination treatment. The agents may be in the same dosage formulations or separate formulations. For combination treatment with more than one active agent, where the active agents are in separate dosage formulations, the active agents can be administered concurrently, or they each can be administered at separate times. The agents may be administered simultaneously or sequentially (i.e., one agent may directly follow administration of the other or the agents may be given episodically, i.e., one can be given at one time followed by the other at a later time, e.g., within a week), as long as they are given in a manner sufficient to allow both agents to achieve effective concentrations in the body. The agents may also be administered by different routes, e.g., one agent may be administered intravenously while a second agent is administered intramuscularly, intravenously or orally. In other words, the co-administration of the opioid receptor antagonist compound with an opioid compound is suitably considered a combined pharmaceutical preparation which contains an opioid receptor antagonist and an opioid compound or agent, the preparation being adapted for the administration of the opioid receptor antagonist on a daily or intermittent basis, and the administration of the opioid agent on a daily or intermittent basis. Thus, the opioid receptor antagonists may be administered prior to, concomitant with, or after administration of the opioids.

Co-administrable agents also may be formulated as an admixture as, for example, in a single formulation or single tablet. These formulations may be parenteral or oral, such as the formulations described in, e.g., U.S. Pat. Nos. 6,277,384; 6,261,599; 5,958,452 and PCT Publication No. WO 98125613, each hereby incorporated by reference. In addition, any mode of administration disclosed herein or known in the art to be compatible with the contemplated co-administration is a suitable mode of administration.

In yet another aspect of the invention, the peripheral opioid receptor antagonist may be co-administered with an opioid or opioid receptor agonist, and another therapeutic agent that is not an opioid or opioid receptor agonist. The opioids and peripheral opioid receptor agonists are described above. Suitable therapeutic agents include anti-biotics and anti-inflammatory agents. The formulations may be prepared using standard formulation methods known to those of skill in the art.

Antibiotics include: Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Anipicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Ceiprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofingin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifirthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine: Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate: Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafingin; Sisomicin; Sisomicin Sulfate; Spariloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin: Vancomycin; Vancomycin Hydrochloride; Virginiamycin; or Zorbamycin.

Antiviral agents include: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime.

Antifungal agents include: Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifumgin Hydrochloride; Parconazole Hydrochloride; Partricin; Potassium Iodide; Proclonol; Pyrithione Zinc; Pyrrolnitrin; Rutamycin; Sanguinarium Chloride; Saperconazole: Scopafungin; Selenium Sulfide; Sinefingin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; or Zinoconazole Hydrochloride.

Anti-inflammatory agents include: Alclofenac; Alclometasone Dipropionate; Algestone Acetonide; Alpha Amylase, Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids or Zomepirac Sodium.

### EXAMPLES

### Example I

### Construction of GFP-PA-I Reporter Strains

A plasmid containing the GFP-PA-I fusion construct was constructed using conventional recombinant DNA techniques. The EGFP gene encoding green fluorescent protein was amplified using the pBI-EGFP plasmid (Clontech) as a template. XbaI and PstI restriction sites were introduced using primers TCTAGAACTAGTGGATCCCCGCGGATG (SEQ ID NO: 1) and GCAGACTAGGTCGACAAGCTTGATATC (SEQ ID NO: 2). The PCR product was cloned directly into the pCR 2.1 vector using a TA-cloning kit (Invitrogen), followed by transformation of the pCR2.1/EGFP construct into E. coli DH5a. The EGFP gene was excised from this construct by digestion with XbaI and PstI and the fragment containing the excised gene was cloned into the E. coli-P. aeruginosa shuttle vector pUCP24, which had been digested with the same restriction enzymes. The resulting construct (i.e., pUCP24/EGFP), containing the EGFP gene in the shuttle vector, was typically electroporated at 25 μF and 2500 V into P. aeruginosa electrocompetent cells. Cells containing pUCP24/EGFP were selected by gentamicin (Gm)challenge, typically at 100 μg/ml. A derivative of pUCP24/EGFP was generated that placed the PA-I lectin/adhesin gene in close proximity to the EGFP gene, effectively linking the genes genetically. In addition to incorporating the structural lecA gene, the construct contained the QS lux box and RpoS consensus sequences in the 5′ non-coding region of lecA, along with rRNA sequence. The derivative construct was termed pUCP24/PLL-EGFP. One of skill would understand how to make and use the above-described construct, as well as other suitable constructs for providing lecA, alone or in physical proximity to a marker gene such as EGFP, using any of a variety of techniques.

### Example 2

### Location of PA-I

PA-I lectin/adhesin was localized to a previously undescribed structural appendage on the outer surface of P. aeruginosa, using conventional techniques.

### Example 3

### Correlation of In Vitro and In Vivo Observations

C. elegans is suitable as an in vivo model system for BSC signaling and its role in the production of PA-I. C. elegans is accepted as a highly accurate and predictable model in which to study the host response to P. aeruginosa. C. elegans worms feed on lawns of P. aeruginosa growing on solid agar and, thus, provides an ideal system in which to study microbial pathogenesis, especially in regard to gut-derived sepsis, since the mode of infectivity is via the digestive tract. These nematodes readily feed on bacteria such as E. coli growing on solid agar plates, yet when fed specific strains of P. aeruginosa, mortality rates exceed 50% within 72 hours. Mortality rates with this model have been shown to be dependent on both the agar environment as well as the strain of P. aeruginosa. Certain strains are highly lethal in this model (e.g., PA14), whereas other strains (PAO1) show intermediate kill rates. The ability to feed C. elegans on lawns of the completely sequenced P. aeruginosa strain PAO1, and selected transposon mutants, while enriching agar plates with various host stress-derived BSCs screened for their ability to express PA-I, makes available a rapid screening system for genes that actively participate in in vivo virulence against the intestinal epithelium. With this approach, the virulence phenotype observed in vitro has been transferred to an in vivo model, with the expectation that results obtained with such a model will prove much more reliable in accurately characterizing the virulence phenotype observed in human patients suffering from an epithelial cell barrier dysfunction.

### Example 4

### In Vitro Recapitulation of the In Vivo “Cues” Released During Surgical Stress

In vitro studies demonstrated that pH, osmolality, and norepinephrine did not change PA-I expression, while opioids, interferon-gamma, C4-HSL, and media from hypoxic and hyperthermia intestinal epithelial cells induced PA-I expression. PA-I was functionally expressed in epithelial cell assays in the presence of the PA-1-inducing compounds.

### Example 5

### Toxin Flux Across Epithelia

Exotoxin A was labeled with AlexaFluor 594, and its transepithelial flux was measured at varying levels of decrease of transepithelial resistance (TEER) of MDCK monolayers that was achieved by apical application of MDCK cells to different concentrations of pure PA-I protein. A five-fold increase in exotoxin A flux across MDCK cells was found when transepithelial resistance was decreased below 50% of control. Purified PA-I decreased the TEER of epithelial cells to the same degree as P. aeruginosa. PA-I null mutants of P. aeruginosa had a significantly attenuated effect on the transepithelial resistance of MDCK cells. Techniques used in conducting the experiments are described in Example 23, below, or are conventional in the art.

### Example 6

### Response of Epithelia to Purified PA-I

The degree of cell polarity (i.e. degree of cell confluency and tight junctional apposition) has been shown to dictate the degree of response to purified PA-I protein. Cells that were loosely confluent had a more profound fall in TEER in response to PA-1 compared to “tighter” and more differentiated cell monolayers. In addition, wounded monolayers exposed dense areas of PA-I binding. Cell culturing was performed as described in Example 24, below; relative confluency was assessed using conventional techniques as would be known in the art.

### Example 7

### Soluble Host Factors Induce Expression of PA-I Lectin/Adhesin

GFP-reporter strains permit demonstrations that virulence gene expression in P. aeruginosa is expressed in vivo within the intestinal tract of a stressed (30% hepatectomy) host. EGPF reporter constructs were specifically designed to contain known upstream regulatory regions involved in PA-1 expression (e.g., lux box (QS promoter elements) and RpoS). The EGFP-PA-I reporter strain, termed PLL-EGFP, was then injected into the cecum of sham-operated (control) mice and mice undergoing surgical hepatectomy. Twenty-four hours later, feces and washed cecal mucosa were then assayed for the presence of fluorescent bacteria. Both within the cecal lumen and in response to contact with the intestinal epithelium, PA-I was expressed in vivo (three- to six-fold over control levels) in response to elements of the local intestinal microenvironment (cecum) of mice subjected to catabolic (surgical) stress. These findings were verified in the non-reporter strain, PA27853, using an assay in which bacterial RNA is extracted from fresh feces using an RNA protection system. Reiterative studies were performed in which PA27853 was introduced into the cecum of control and hepatectomized mice and then bacterial RNA recovered from fresh feces 24 hours later for quantitative RT-PCR (QRT-PCR) of both PA-I and exotoxin A (about 600% and 800% respectively). This assay provides a precise molecular “snapshot” of the effect of the in situ cecal environment on P. aeruginosa virulence gene expression. Results demonstrated that the cecal microenvironment of a stressed host induced PA-I and exotoxin A virulence gene expression. Next, in order to determine whether these findings were due to soluble factors released into the intestinal lumen, particulate-free filtrates were prepared from cecal luminal contents from control and hepatectomized mice and added to fresh cultures of the reporter strain PLL-EGFP. Results demonstrated that when PA-I GFP reporter strains were exposed to filtered cecal contents from mice exposed to surgical hepatectomy, a 248%±12 increase in fluorescence was observed compared to 112%±15 for filtered cecal contents from sham-operated mice (P<0.001). These results indicated that a soluble factor is present in the intestinal lumen following surgical stress that activates PA-I expression. Two remaining issues included, first, whether the soluble PA-1-inducing components are generated from within the intestinal tract itself or from the systemic compartment and, second, whether the soluble PA-1-inducing components are specific to hepatectomy-induced stress. To address these issues an animal model of segmental intestinal ischemia was developed in which an isolated loop of intestine (6 cm, proximal ileum) was luminally cannulated and timed aliquots of luminal perfusates were collected following 10 minutes of ischemia followed by 10 minutes of reperfusion. Blood was then obtained at the end of the experiment in order to determine the effect of systemic factors on PA-I expression. The results indicated that 1) intestinal ischemia, similar to hepatectomy, can release soluble factors into the intestinal lumen capable of signaling P. aeruginosa to express PA-I; 2) these factors may originate from the intestinal tract itself, since during ischemia the intestine is isolated from systemic factors; 3) blood components do not induce PA-I expression; and 4) the presence of the normal flora, virtually absent in flushed small bowel segments, appears to play no role in this response. To rule out the possibility that the in vivo expression of PA-I was due to secondary effects of surgical stress on physico-chemical changes in the local microenvironment, P. aeruginosa strain PA-27853 and reporter strains (PLL-EGFP) were exposed to ambient hypoxia (0.3% O₂), pH changes (6-8), and 80% CO₂. None of these conditions induced PA-I expression. In addition, reporter strains exposed to the blood or liver tissue of mice following sham-operation or hepatectomy, did not display enhanced fluorescence. These studies suggest that bacterial signaling components released in response to surgical and ischemic stress are highly concentrated in the intestinal tract and are generated by host-cell derived factors that can be isolated from, and detected within, the intestinal lumen. Based on these results, it is expected that any form of stress (e.g., surgery, injury such as traumatic injury, illness, heat, starvation, hypoxia, and the like) to epithelial cells, such as intestinal epithelial cells, will typically lead to a change in the level of at least one soluble factor involved in bacterial signaling, i.e., at least one soluble BSC.

### Example 8

### Bacterial Signaling Compounds (BSCs) Inducing PA-I Lectin/Adhesin Expression are Found in Epithelial Cells

Using Caco-2 intestinal epithelial cells, the issue of whether components of intestinal epithelial cells themselves played a role in triggering the expression of PA-I was addressed. Strain PA27853 was exposed to media (apical and basolateral) and Caco-2 cell fractions (cytosolic, nuclear, membrane) at various time intervals. PA-I mRNA was measured in PA27853 in response to the various Caco-2 cell media fractions in the presence and absence of GalNac, a sugar that binds specifically to PA-I and prevents P. aeruginosa adherence to Caco-2 cells. Media alone from Caco-2 cells grown in transwells (apical or basolateral) had no effect on PA-I expression. However, Caco-2 cell membrane fractions triggered the accumulation of a very high abundance of PA-I mRNA (>10 fold increase)-an effect that was attenuated in the presence of GalNac. These in vitro findings are in agreement with the above mouse studies showing that PA-I can be activated in response to contact with the intestinal epithelium, yet in the unstressed Caco-2 cell system, luminal contents (apical media) had no effect, similar to the control mice. Experiments in which PA27853 were inoculated onto the apical surface of Caco-2 cells and allowed to densely adhere (extended culture), demonstrated an increase in PA-I mRNA, which was nearly completely abolished in the presence of GalNac. Thus, PA-I expression is influenced by both membrane-bound and soluble factors, and it is contemplated that modulators of the bacterial signaling process include, but are not limited to, effectors (i.e., enhancers, activators, and inhibitors) of a soluble factor, a membrane-bound factor, or both.

### Example 9

### Stressed Caco-2 Cells Release Soluble Factors that Induce PA-I Lectin/Adhesin Expression

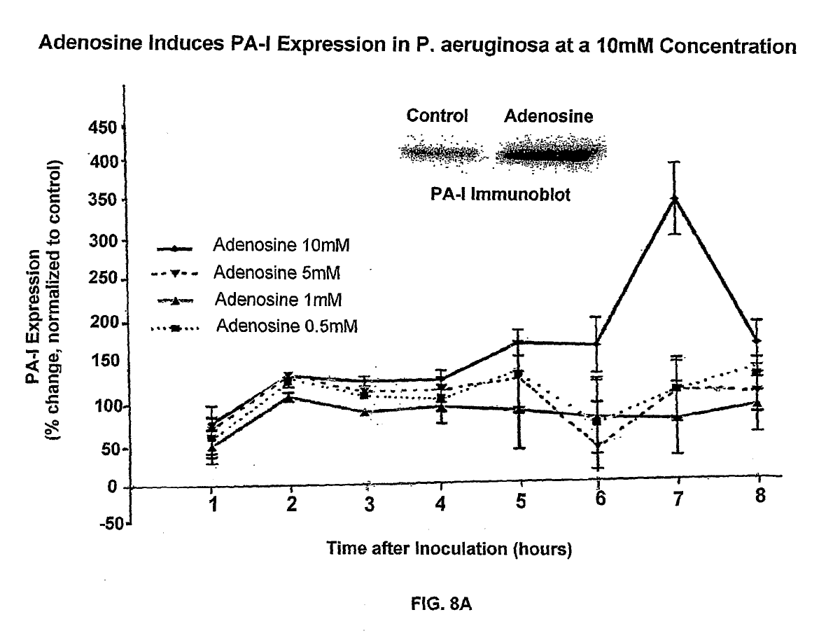

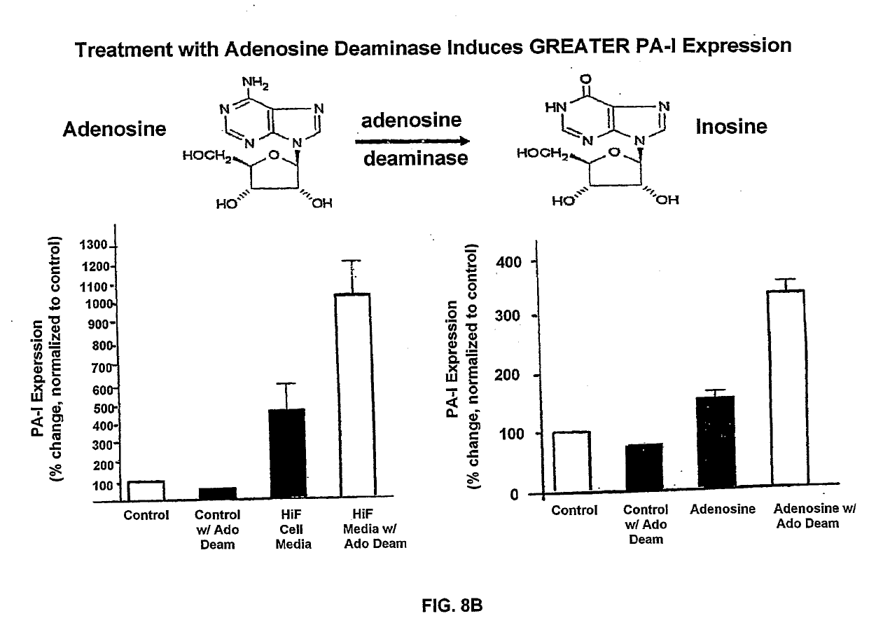

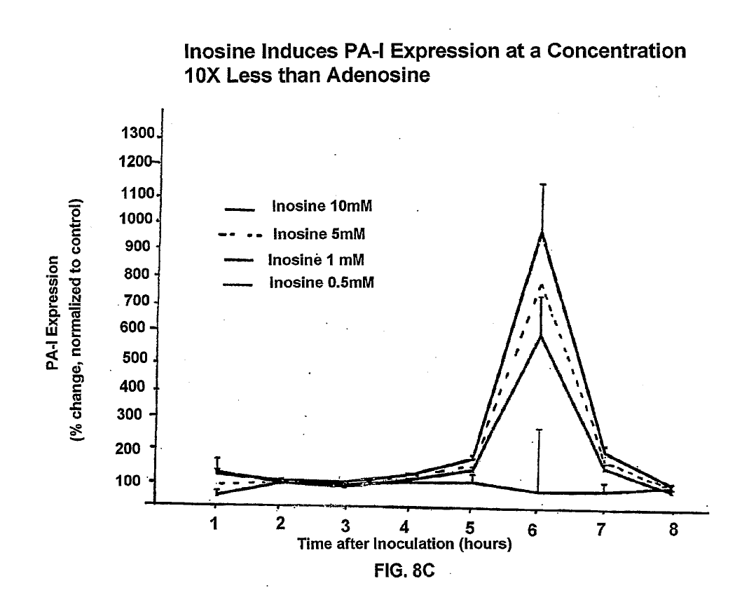

In order to recapitulate the type of stress that the intestinal epithelium is exposed to under conditions of surgical injury, a confluent monolayer of Caco-2 cells was subjected to hypoxic stress (1 hour 0.3% hypoxia+30 minutes normoxic recovery). A PA-I GFP reporter strain, PLL-EGFP, was then exposed to the apical media from stressed and non-stressed cells. The results demonstrated a rapid and significant increase in PA-I promoter activity in these strains based on relative fluorescence units (RFU's) of PLL-EGFP. Results were confirmed by Northern blot analysis. Analysis of the spatial and temporal dynamics of these experiments was carried out using fluorescent microscopy. In hypoxic cells, contact-induced expression of PA-I promoter activity was observed and demonstrated preferential adherence of bacteria to the tri-cellular junctions of Caco-2 cells (FIG. 8B). Reiterative experiments exposing Caco-2 cells to heat shock stress (42° C. 1 h+2 h recovery) demonstrated similar findings to hypoxia. A near ten-fold increase in fluorescence was observed in the PA-I GFP reporter strain exposed to apical media from heat shock stressed Caco-2 cells. Membrane fractions from both hypoxic and heat shock stressed Caco-2 cells induced extremely high PA-I expression (approximately 100 fold) that could not be quantifiably distinguished between groups.

Media from hypoxic and heat shock stressed Caco-2 cells were next fractionated into 5 molecular weight fractions (<3, 3-10, 10-20, 20-30, >30 kDa) using centricones, to determine if a specific MW fraction could be identified that induces PA-I expression. In addition, to determine if the bacterial signaling compound (s) was a protein, fractions were treated with heat inactivation and the protein inhibitor, proteinase K. For the hypoxic media the identified fraction was 10-30 kD and for the heat shock fraction the identified fraction was 30-50 kD. Both fractions were inactivated, consistent with the BSC being proteins. Data from these experiments strongly suggest that there are two distinct bacterial signaling compounds released into the apical media in response to hypoxic and heat shock stress in Caco-2 cells that are proteins (peptides). These findings are significant because 1) the fractionated compounds are soluble and can be mass produced in unlimited supply by growing large sheets of Caco-2 cells, and 2) the compounds are proteins and therefore can be easily characterized by mass spectrometry and identified. Although more highly purified and characterized factors will facilitate technological development, screens for modulators of the activity (e.g., bacterial signaling activity) of such factors are presently available, with variations on a given screening methodology apparent to one of ordinary skill using no more than routine procedures.

Stimulated Immune Cells Release Factors that Induce PA-I Lectin/Adhesin Expression

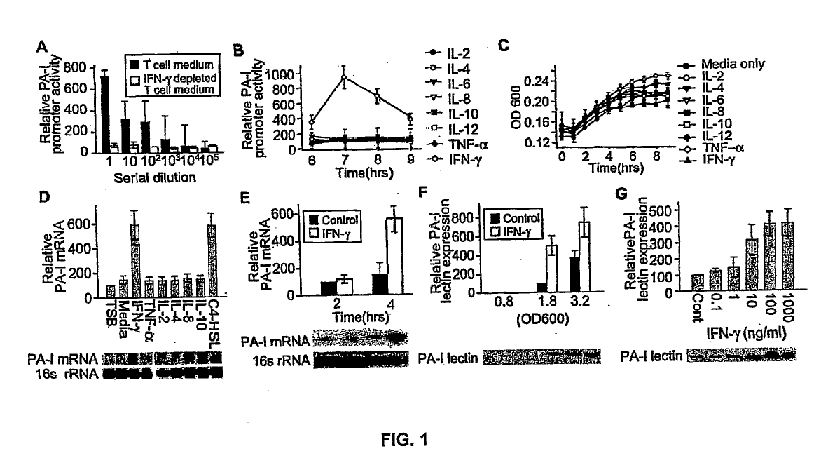

Immune elements released at the mucosal epithelial surface, the primary site of colonization for P. aeruginosa, were considered to be suitable candidates to serve as host stress-derived bacterial signaling compounds. As a physiologically relevant in vitro system to determine whether immune factors can activate P. aeruginosa virulence, supernatants from antigen-stimulated T cells were evaluated for their ability to increase PA-I expression in the P. aeruginosa strain PLL-EGFP/27853, which carries a PA-1-GFP reporter construct. P. aeruginosa cells were incubated with supernatants from stimulated T-cells and PA-I expression was assessed by GFP expression levels (fluorescence). Media from activated T cells, which release a comprehensive array of cytokines (D. J. Schwartzentruber, S. L. Topalian, M. Mancini, S. A. Rosenberg, J Immunol 146, 3674 (May 15, 1991)), induced PA-I expression as assessed by enhancement of fluorescence in the PA-1-GFP fusion reporter strain (L. Wu et al., Gastroenterology 126, 488 (February, 2004)) (FIG. 1A).

To determine whether this effect was due to cytokines, the reporter strain was exposed to various cytokines (human IL-2, IL-4, IL-6, IL-8, IL-10, IL-12, Interferon gamma (IFN-γ) and tumor necrosis factor alpha (TNF-α) with only IFN-γ showing a significant increase in PA-1 expression beginning at early stationary phase of growth (FIG. 1C). None of the cytokines tested had any significant effect on bacterial growth (FIG. 1B). To test whether IFN-γ was required in the media of activated T-cells to enhance PA-I expression, we depleted IFN-γ from the culture media of activated T cells using specific antibody. Immunodepletion of the media of IFN-γ resulted in the complete loss of its PA-I inducing capacity (FIG. 1A), suggesting that IFN-γ is essential for PA-I expression in this system. To further confirm the role of IFN-γ as a host stress-derived bacterial signaling compound, we exposed the completely genomically sequenced strain of P. aeruginosa, PAO1 (C. K. Stover et al., Nature 406, 959 (Aug. 31, 2000)), to human recombinant IFN-γ, TNF-α, and various other cytokines (IL-2, IL-4, IL-8, IL-10) and measured lecA (encoding for PA-I) mRNA by Northern blot. IFN-γ, but not TNF-α or other cytokines, induced lecA mRNA (FIG. 1D). These data indicated that human IFN-γ functions as a host cell-derived bacterial signaling molecule to which P. aeruginosa responds with enhanced virulence.

### Example 10

### Identification of Host Stress-Derived BSCs by Screening Candidate Agents: the Role of Cytokines

As a method to rapidly identify host BSCs, P. aeruginosa strains were exposed to media containing adenosine (released by Caco-2 cells in response to hypoxia) TNFα, IL-2, IL-6 IL-8 (released by epithelia in response to bacterial invasion/ischemia), and IFNγ (released by intraepithelial lymphocytes in response to bacterial invasion/ischemia). In addition, strains were exposed to apical media from Caco-2 cells basolaterally exposed to single and combinations of the various epithelial-derived cytokines. Dr. Jerrold Turner, has demonstrated that basolateral exposure of Caco-2 cells to the combination of IFN γ and TNF a activates cellular signaling proteins that dramatically alter tight junctional proteins and function. Media from Caco-2 cells exposed to various combinations of these cytokines had no effect on PA-I expression. However, IFN-γ alone induced a direct effect on PA-I expression while none of the other compounds alone had any effect. Another issue was whether IFN γ binding to P. aeruginosa could be demonstrated for strain PA27853. Using both ELISA, immunofluorescence microscopy, and flow cytometry, the binding characteristics of IFNγ were determined for both whole bacteria and membrane fractions of P. aeruginosa. Results demonstrated that IFN-γ showed high binding affinity to whole bacterial cells of PA27853. These effects were also observed with strain PAO1. Next, solubilized and separated membrane proteins of P. aeruginosa (P 7853) were solubilized and separated, which showed that IFN-γ avidly binds to a single 30 kDa protein band. It has been difficult to immunoprecipitate a significant quantity of this protein from PA27853, but it has been determined that this protein can also be immunoprecipitated from E. coli. Next, IFN-γ binding specificity, to whole bacterial cells, was determined, using reiterative binding studies in the presence of various gram-negative bacterial strains, including P. aeruginosa. Multiple strains of bacteria displayed IFN-γ binding by ELISA binding assays suggesting that an IFN-γ binding site may be conserved across a wide variety of procaryotic cells. Finally, in order to determine if PA-I was functionally expressed in PA2±853 in the presence IFN-γ, PA27853 was inoculated onto Caco-2 cell monolayers in the presence of IFN-γ and the effect on barrier dysregulating dynamics of PA27853 against this cell line were assessed to determine if IFN-γ shifted the dynamics. IFN-γ enhanced the barrier dysregulating effect of PA27853 against the intestinal epithelium after five hours of incubation by about 20%. Thus, cytokines such as IFN-γ are embraced by the invention as effective modulators of bacterial signaling and, ultimately, of eukaryotic (e.g., epithelial) cell barrier function.

The expression of virulence in P. aeruginosa is highly regulated by the quorum sensing signaling system (QS), a hierarchical system of virulence gene regulation that is dependent on bacterial cell density and hence growth phase (M. Whiteley, K. M. Lee, E. P. Greenberg, Proc Natl Acad Sci U SA 96, 13904 (Nov. 23, 1999)) (S. P. Diggle, K. Winzer, A. Lazdunski, P. Williams, M. Camara, J Bacteriol 184, 2576 (May, 2002)). Therefore in order to determine the effect of growth phase on the response of P. aeruginosa to IFN-γ, bacteria were harvested at various growth phases following exposure to IFN-γ, and PA-I mRNA and protein measured by Northern blot and immunoblot respectively. Both PA-I mRNA and protein were increased in response to IFN-γ at early stationary phase of growth (FIG. 1E, 1F). PA-I protein expression in PAO1 was also dose dependent, with the greatest increase seen with 100 ng/ml (FIG. 1G). Taken together these results suggested the exposure of P. aeruginosa to IFN-γ enhanced PA-I expression but was not able to shift its expression to an earlier phase of growth.

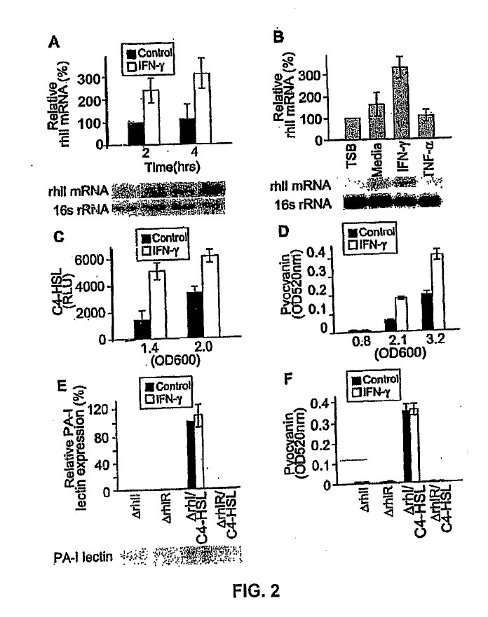

To determine whether IFN-γ induced PA-I via activation of the quorum sensing signaling system, we measured rhlI gene expression in PAO1 in response to IFN-γ by Northern blot. IFN-γ induced rhl1 transcription in PAO1 (FIG. 2A, 2B). RhlI is the gene required for the synthesis of C₄-HSL (C₄-homoserine lactone), a core quorum sensing signaling molecule that plays a key role in the expression of PA-I (M. R. Parsek, E. P. Greenberg, Proc Natl Acad Sci (USA) 97, 8789 (Aug. 1, 2000)). We next determined if exposure of P. aeruginosa to IFN-γ would lead to the synthesis of C₄-HSL. PAO1 was exposed to 100 ng/ml of IFN-γ and C₄-HSL measured in bacterial supernatants. C₄-HSL synthesis was increased in PAO1 exposed to IFN-7 (FIG. 2C). To verify that activation of the QS system by IFN-γ led to the production of other QS-dependent virulence products, we measured pyocyanin production, a redox active compound, in PAO1 at various phases of growth following exposure to IFN-γ and showed that IFN-γ increased pyocyanin production in PAO1 (FIG. 2D). Finally, to determine whether rhlI and rhlR are required for the production of pyocyanin (PCN) and PA-I expression in response to IFN-γ, an rhlI mutant P. aeruginosa strain and, independently, an rhlR⁻ mutant P. aeruginosa strain were exposed to IFN-γ. PCN production and PA-I expression induced by IFN-γ were abolished in these mutant strains (FIG. 2E, 2F). These data suggest that the QS system plays a key role in the response of P. aeruginosa to IFN-γ.

### Example 11

### Interferon-γ Binds to the Surface of P. aeruginosa

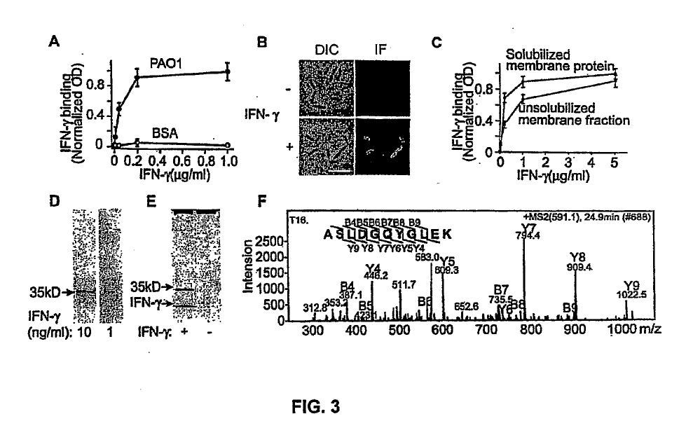

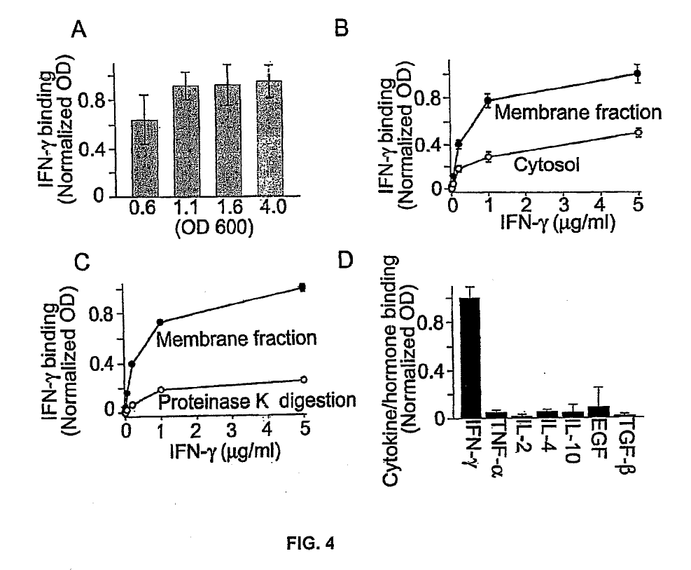

IFN-γ direct binding to a protein on the surface of P. aeruginosa, in the course of virulence activation, was also investigated. ELISA binding assays were performed by first coating microtiter plates with P. aeruginosa (strain PAO1), then adding recombinant human IFN-γ (rH IFN-γ), followed by biotin-labeled anti-IFN-γ antibody. IFN-γ avidly bound to whole fixed cells of P. aeruginosa in a dose-dependent manner (FIG. 3A). The ELISA data were confirmed by the results of immunofluorescent imaging of bacterial cells exposed to IFN-γ followed by biotin-labeled anti-IFN-γ antibody and Alexa 594-labeled streptavidin. The vast majority of bacterial cells (73%±3.2% vs. 8.5%±2.5%) bound IFN-γ (FIG. 3B). The binding capacity of the IFN-γ to the P. aeruginosa was affected by bacterial growth phase (FIG. 4A). In order to localize the binding site of IFN-γ to P. aeruginosa (PAO1), equal protein concentrations of membrane and cytosol fractions of P. aeruginosa were prepared and coated onto ELISA microtiter plates. ELISA binding assays showed that IFN-γ preferentially bound to membrane fractions of P. aeruginosa (FIG. 4B). To determine if the observed membrane binding by IFN-γ was protein dependent, membrane fractions were treated with proteinase K for 3 hours and IFN-γ binding assessed. Binding by IFN-γ to P. aeruginosa membranes after treatment with proteinase K was decreased (FIG. 4C) suggested that IFN-γ binds to protein on the bacterial cell membrane. We next determined if other cytokines similarly would bind to P. aeruginosa cell membranes by performing reiterative binding studies with human TNF-α, IL-2, IL-4, IL-10, EGF, and TGF-β. No binding was observed with any of these cytokines (FIG. 4D). Taken together these data indicate IFN-γ bound to membrane protein on P. aeruginosa.

To isolate the putative protein to which IFN-γ binds on the cell membrane of P. aeruginosa, membrane proteins solubilized with mild detergents were initially shown to retain their binding capacity to IFN-γ by ELISA (FIG. 3C). Prior to isolation of the putative binding protein of IFN-γ, we sought to determine whether IFN-γ bound to single or multiple membrane proteins. Membrane proteins were then separated by non-denaturing gel electrophoresis, transferred to PVDF membranes and hybridized with IFN-γ followed by biotin-labeled anti-IFN-γ antibody. Results demonstrated a single immunoreactive band of about 35 kD. Immunoreactivity was IFN-γ dose-dependent (FIG. 3D). In order to identify the putative binding protein, membrane protein was extracted from 4 L of freshly grown P. aeruginosa and fractionated by molecular weight between 10-100 kD. Solubilized protein was then immunoprecipitated using IFN-γ and anti-IFN-γ antibody. BSA was used as a control. Immunoprecipitation resulted in the appearance of a distinct protein with a molecular weight of about 35 kD. To further confirm that the protein isolated by immunoprecipitation was dependent on the presence of IFN-γ, equally divided solubilized membrane protein fractions were mixed with and without IFN-γ and then immunoprecipitated with anti-IFN-γ antibody. The 35 kD band appeared only in the solubilized membrane protein mixed with IFN-γ (FIG. 3E). The IFN-γ-dependent band was identified by ESI-TRAP-Electrospray LC-MSMS Ion Trap as the P. aeruginosa outer membrane porin OprF (FIG. 3F). These data established that IFN-γ binds to the P. aeruginosa outer membrane protein OprF (A. O. Azghani, S. Idell, M. Bains, R. E. Hancock, Microb Pathog 33, 109 (September, 2002)).

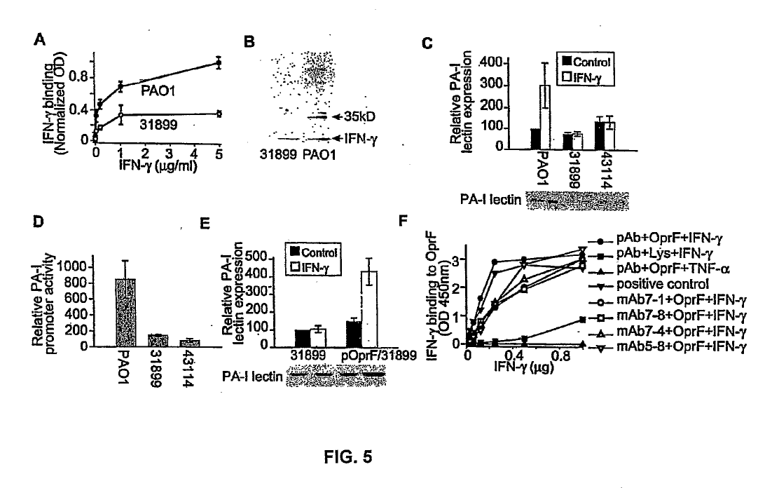

To verify that OprF represented the major binding site for IFN-γ in P. aeruginosa strain PAO1, solubilized membrane proteins from OprF knockout strains of P. aeruginosa strain PAO1 (M. A. Jacobs et al., Proc Natl Acad Sci USA 100, 14339 (Nov. 25, 2003)) were tested for their ability to bind IFN-γ in comparison to the wild-type strain using the established ELISA and immunoprecipitation technique. ELISA binding assays of solubilized membrane proteins demonstrated reduced binding of IFN-γ in OprF⁻ strains (FIG. 5A). Immunoprecipitation of solubilized membrane protein using IFN-γ and specific antibody confirmed the role of OprF by showing complete loss of the approximately 35 kD band in the OprF mutant strain (FIG. 5B). To verify the functional role of OprF on the responsiveness of P. aeruginosa to IFN-γ, we examined the expression of the PA-I protein in wild-type and OprF mutant strains exposed to 100 ng/ml of IFN-γ. Results demonstrate that mutant strains failed to increase the expression of the PA-I protein in response to an effective stimulating dose of IFN-γ as compared to the wild-type strain (FIG. 5C). The results from reporter gene fusion of wild-type and OprF mutant strains also demonstrated that IFN-γ activated PA-I expression through OprF (FIG. 5D). To further verify the role of OprF, OprF was reconstituted in mutant P. aeruginosa strain 31899 using the plasmid pUCP24/OprF. Reconstituted strains demonstrated recovery of their responsiveness to IFN-γ with an increase in PA-I protein expression (FIG. 5E). Finally, we verified the binding between OprF and IFN-γ by showing that purified OprF directly binds human IFN-γ (FIG. 5F) in a dose-dependent manner.

### Example 12

### Identification of Host Stress-Derived BSCs by Screening Candidate Agents: the Role of Endogenous Opioids

Although it was known that the counter-regulatory hormone, norepinephrine, increased the binding of P. aeruginosa to human O erythrocytes, there has been no information relating to the involvement of PA-I in the process. Accordingly, an assay to detect the presence of extracellular PA-I was performed. It was possible that norepinephrine would function as a host BSC for P. aeruginosa and, thus, affect human O erthyrocytes in a manner similar to the way it affected E. coli. Despite extensive analyses, PA-I expression was not affected by this compound. The screening of other catecholamines, all without positive results, led to the expectation that opioids, particularly morphine alkaloids, would activate PA-I. Endogenous morphine has been documented to be released in direct proportion to the magnitude of surgical stress/injury in both animals and humans. Initially, morphine was assessed for its effects. Interestingly, exposure of Pseudomonas strain PA27853 to physiologic concentrations of morphine (13 μM) resulted in a four-fold increase in PA-I expression (in comparison, in the same assay C4-HSL induced about a 16-fold increase in PA-I expression). As morphine is considered to be a non-selective opioid, specific endogenous opioid agonists with high selective affinity to μ, κ and δ receptors were tested for their abilities to induce PA-I lectin/adhesin expression in strains PA27853 and PAO1. Also tested were two pure μ peptide agonists, endomorphine-1 (E1) (Tyr-Pro-Trp-Phe-NH₂; SEQ ID NO:24) and endomorphine-2 (E2) (Tyr-Tyr-Pro-Phe-Phe-NH₂; SEQ ID NO:25), the potent κ opioid non-peptide agonist U-50488, and the potent δ opioid non-peptide agonist BW373U86 for their respective abilities to induce PA-I expression in the reporter strain P. aeruginosa PA27853/PLL-EGFP. Results demonstrated that agonists targeting the κ and δ receptors had the greatest effect on PA-I expression as judged by increased fluorescence of the GFP reporter strain. In order to determine if PA-I was functionally expressed when exposed to the various opioid agonists, the agonists were tested for their abilities to shift the barrier-dysregulating dynamics of PA27853 in MDCK cells. Results show that all three of the opioids that induced PA-I expression (morphine, κ and δ agonists), shifted the virulence of PA7853 as judged by a more profound decrease in the TEER of MDCK cells following apical exposure (about 15%, 20%, and 25% additional TEER decrease, respectively).

In order to determine if morphine could shift the in vivo virulence of P. aeruginosa, mice were implanted with slow release morphine pellets that release a daily dose of morphine that is similar to that used clinically (pellets obtained from the National Institute on Drug Abuse (NIDA). Control mice were implanted with a placebo pellet. Mice drank infant formula spiked with a daily inoculum of 1×10⁸ cfu/ml of PA27853. All the morphine treated mice developed severe sepsis (4/4) and significant mortality while none of the control mice appeared septic and all survived. Finally, agonists were tested for their ability to induce biofilm in PA27853, a quorum sensing dependent phenotype. Biofilm production by P. aeruginosa and other organisms has been established to be a major phenotype indicative of enhanced virulence. The opioid κ and δ agonists significantly increased biofilm production in strains PA27853, about 150% and 180% of PA27853 induction respectively. Taken together, these studies demonstrate that opioid agonists can directly influence the virulence, and potential lethality, of P. aeruginosa. It is expected that opioid agonists and antagonists, whether found endogenously or not, and whether purified from a natural source, chemically synthesized, or produced by a combination thereof, are contemplated by the invention as useful modulators of the bacterial signaling affecting microbial pathogenesis generally, and eukaryotic (e.g., epithelial or endothelial) cell barrier function more specifically.

### Example 13

### Role of κ-Opioids in P. aeruginosa Virulence Expression

Opioid compounds, known to accumulate in tissues such as the lung and intestine following stress, directly activate the virulence of P. aeruginosa as judged by pyocyanin production, biofilm formation, and the expression of the PA-IL protein. Specifically, pyocyanin production was enhanced in the presence of the selective κ-opioid receptor agonist, U-50,488, and the naturally occurring endogenous peptide dynorphin, also a selective κ-opioid receptor agonist. To understand the regulatory pathway(s) involved in opioid-induced virulence gene expression in P. aeruginosa, the effect of U-50,488 on multiple mutant P. aeruginosa strains defective in key elements involved in pyocyanin production was examined. Results demonstrated that the global transcriptional regulator, MvfR, plays a key role in pyocyanin production in response to U-50,488. Intact MvfR was also shown to be required for P. aeruginosa to respond to C4-HSL, a key quorum sensing signaling molecule known to activate hundreds of virulence genes. Taken together, these studies indicate that opioid compounds serve as host-derived signaling molecules that can be perceived by bacteria during host stress for the purposes of activating their virulence phenotype.

Bacterial strains and culture conditions. P. aeruginosa strains PAO1 and 27853, and their derivative strains (Table 1) were routinely grown in tryptic soy broth (TSB) supplemented when necessary with tetracycline (Tc), 60 μg/ml, and/or gentamicin (Gm), 100 μg/ml. Alkaloid opiates morphine, a preferable μ-opioid receptor agonist (A. Shahbazian, et al., Br J Pharmacol 135, 741 (2002)), U-50,488, a specific κ-opioid receptor agonist (J. Szmuszkovicz, Prog Drug Res 53, 1 (1999)), and BW373U86, a specific δ-opioid receptor agonist (S. F. Sezen, V. A. Kenigs and D. R. Kapusta, J Pharmacol Exp Ther 287, 238 (1998)), along with the peptide opioid dynorphin, a specific κ-opioid receptor agonist (Y. Zhang, E. R. Butelman, S. D. Schlussman, A. Ho and M. J. Kreek, Psychopharmacology (Berl) 172, 422 (2004)), and specific κ-opioid-receptor antagonist nor-binaltorphimine (A. Shahbazian, et al., Br J Pharmacol 135, 741 (2002)) were used in the experiments. Morphine was purchased from Abbott Laboratories, U-50,488, BW373U86, dynorphin, nor-binaltorphimine, and methyl anthranilate from Sigma-Aldrich, and C4-HSL from Fluka.

Complementation of MvfR mutant with mvfR gene. Amplified mvfR was directly cloned in pCR2.1 (Invitrogen), digested with XbaI-HinDIII restriction endonucleases and subcloned into pUCP24 under the Plac promoter to create pUCP24/mvfR. The plasmids pUCP24 (blank control) and pUCP24/mvfR were electroporated in strain 13375, defective in MvfR production, to create the P. aeruginosa strain 13375/MvfR (Tables 1, 2).

Complementation of GacA mutant with gacA gene. The gacA gene, a member of a two-component signaling method involved in the elaboration of virulence in many gram-negative bacteria, was amplified and directly cloned into pCR2.1 (Invitrogen). The gene was then excised with XbaI-HinDIII restriction endonucleases and subcloned into pUCP24 under the Plac promoter to create pUCP24/gacA. The plasmids pUCP24 (blank control) and pUCP24/gacA were electroporated in P. aeruginosa strain PA06281, defective in GacA production, to create the P. aeruginosa strain PAO6281/GacA (Tables 1, 2).

Truncation of MvfR. PCR products of truncated mvfR genes amplified from pUCP24/MvfR and their respective primers (Tables 1, 2) were purified using a Geneclean kit (Qbiogene), digested with XbaI-HinDIII restriction endonucleases, and ligated into pUCP24 followed by electroporation into P. aeruginosa strain 13375.

Pyocyanin assay. Bacteria were grown in TSB at 37° C. under shaking conditions at 220 rpm, with opioid compounds added at the early exponential phase of bacterial growth (OD_{600 nm} of about 0.15-0.2). After incubation, pyocyanin was extracted from culture media in 6 chloroform followed by re-extraction in 0.2 M HCl, and measured at OD₅₂₀ nm as described (D. W. Essar, L. Eberly, A. Hadero and I. P. Crawford, J Bacteriol 172, 884 (1990)).

PA-IL assays. Immunoblotting and fluorescence of the GFP-PA-IL reporter strain were used to determine the effect of opioids on PA-IL expression. For immunoblotting, P. aeruginosa PAO1 was grown in TSB media with or without 100 μM U-50,488, and cells were collected at the late exponential phase of growth (OD600 nm=1.8). Equal amounts of protein from each sample were separated by 15% SDS-PAGE, transferred to a PDF membrane, and probed with affinity-purified rabbit polyclonal anti-PA-IL antibodies. The probed membranes were treated with anti-rabbit horseradish peroxidase-conjugated IgG, and developed using SuperSignal West Femto chemiluminescent substrate (Pierce). For PA-IL expression detected by fluorescence, a bacterial culture of the GFP-PA-IL reporter strain 27853/PLL-EGFP (L. Wu, et al., Gastroenterology 126, 488 (2004)) was plated at a final concentration of 108 CFU/ml in 96-well fluorometry plates (Costar) in conventional media, i.e., HDMEM media containing 10% FBS and HEPES buffer with or without 60 μM of U-50,488. Incubation was performed at 37° C., 100 rpm, and fluorescence reading was performed hourly with a 96-well fluorometry Plate Reader (Synergy HT, Biotec Inc.) at excitation/emission of 485/528 nm. Fluorescence intensity was normalized to cell density measured at 600 nm.

Biofilm formation assay. Bacterial cells were plated in quadruplicate in 96-well U-bottom plates (Falcon) at a concentration of 107 CFU/ml in M63S media (13.6 g KH2PO4 1-1, 2.0 g (NH4)2SO4 1-1, 0.5 mg FeSO4x7H2O 1-1), supplemented with 0.5% casamino acids, 1 mM MgSO4x7H20 and 0.2% glucose, and incubated overnight at 37° C. under static conditions. U-50,488 was added at the inoculation point. After inoculation, the wells were rinsed thoroughly with water and the attached material was stained with 0.1% crystal violet, washed with water, and solubilized in ethanol. Solubilized fractions were collected and absorbance measured at 590 nm as described (G. A. O'Toole and R. Kolter, Mol Microbiol 28, 449 (1998)) with a Plate Reader.

κ-Opioid Receptor Agonists U-50,488 and Dynorphin Stimulate Pyocyanin Production in P. aeruginosa.

P. aeruginosa harvested from the intestine of surgically stress mice appeared intensely green compared to P. aeruginosa from the intestines of sham-operated control mice. Thus, P. aeruginosa might be responding to a signal to produce increased amounts of pyocyanin (PCN) in response to environmental cues unique to the intestinal tract of stressed mice. Pyocyanin, a redox active compound that increases intracellular oxidant stress, has been shown to play a key role in the virulence of P. aeruginosa in animal models mediating tissue damage and necrosis during lung infection (G. W. Lau, H. Ran, F. Kong, D. J. Hassett and D. Mavrodi, Infect Immun 72, 4275 (2004)). P. aeruginosa PAO1 was exposed to peptide opioids and alkaloid opiates representing groups of μ-, κ-, and δ-opioid receptor agonists. Results indicated that following overnight exposure, the alkaloid opiate U-50,488, a specific κ-opioid receptor agonist, induced an intensely bright green color in P. aeruginosa PAO1, while no such effect was observed with any of the remaining compounds. To verify that the color change was due to PCN production, pyocyanin was measured at OD520 nm (D. W. Essar, L. Eberly, A. Hadero and I. P. Crawford, J Bacteriol 172, 884 (1990)). Results demonstrated that U-50,488 induced a dose-dependent effect on PCN production that was observed with P. aeruginosa strains PAO1 and 27853. Exposure of P. aeruginosa to dynorphin, a naturally occurring specific κ-opioid receptor peptide agonist, also enhanced PCN production in a dose-dependent manner. Reiterative experiments performed in the presence of the specific κ-opioid receptor antagonist norbinaltorphimine (NOR), demonstrated that NOR attenuates enhanced PCN production in PAO1 following exposure to U-50,488 in a dose-dependent manner and completely inhibits enhanced PCN production at a dose of 200 μM.

The κ-Opioid-Receptor Agonist U-50,488 Shifts Pyocyanin Production at Lower Cell Densities in P. aeruginosa.

We assessed the dynamics of PCN production in response to U-50,488 at varying cells densities, since the expression of QS-dependent genes is known to occur at high bacterial cell densities when QS signaling molecules reach their threshold concentrations. As a positive control, bacteria were exposed to C4-homoserine lactone (C4-HSL), a QS signaling molecule involved in PCN regulation (M. R. Parsek and E. P. Greenberg, Proc Natl Acad Sci USA 97, 8789 (2000)). We found that exposure of PAO1 to U-50,488 had a similar effect to exposure of cells to C4-HSL, resulting in a shift in the production of PCN at lower cell densities. Neither compound had an effect on bacterial growth in TSB media.

The κ-Opioid-Receptor Agonist U-50,488 Exerts its Inducing Effect on Pyocyanin Production Via Elements of the Quorum Sensing System in Pseudomonas aeruginosa.

The pathways of PCN regulation and biosynthesis have been described in detail (D. V. Mavrodi, et al., J Bacteriol 183, 6454 (2001), E. Deziel, et al., Proc Natl Acad Sci USA 101, 1339 (2004), T. R. de Kievit, Y. Kakai, J. K. Register, E. C. Pesci and B. H. Iglewski, FEMS Microbiol Lett 212, 101 (2002), S. L. McKnight, B. H. Iglewski and E. C. Pesci, J Bacteriol 182, 2702 (2000)). In order to define potential pathways by which U-50,488 induces PCN production, mutant strains defective in key genes involved in PCN production were exposed to U-50,488 and the effect on pyocyanin production was measured. First, mutants defective in genes encoding core elements of the QS system (J. P. Pearson, E. C. Pesci and B. H. Iglewski, J Bacteriol 179, 5756 (1997)) (lasR, lasI, rhlI, rhlR) were analyzed and the results demonstrated that exposure to U-50,488 did not restore PCN production (relative to non-mutant strains) in any of these mutants. The roles of the global virulence regulators GacA and MvfR on PCN production were then investigated. Both GacA (C. Reimmann, et al., Mol Microbiol 24, 309 (1997)) and MvfR (E. Deziel, et al., Proc Natl Acad Sci USA 101, 1339 (2004)) have been shown to play a major role in PCN production in P. aeruginosa. Neither ΔGacA nor ΔMvfR produced PCN, as expected, and exposure to U-50,488 could not restore PCN production. C4-HSL was also unable to restore PCN production in the gacA and mvfR mutants. The finding that C4-HSL did not restore PCN production in the GacA mutant is consistent with the finding that the analogous QS molecule, N-hexanoyl-HSL (C6-HSL), did not restore phenazine production in a ΔGacA mutant of P. aureofaciens (S. T. Chancey, D. W. Wood and L. S. Pierson, 3rd, Appl Environ Microbiol 65, 2294 (1999)). Seven additional mvfR mutants from the comprehensive transposon library (M. A. Jacobs, et al., Proc Natl Acad Sci USA 100, 14339 (2003)) (i.e., numbers 8902, 47418, 35448, 51955, 21170, 47853, and 47198) were exposed to C4-HSL in order to confirm this finding. Results demonstrated that none of these mutants produced PCN in the presence of 1 mM C4-HSL.

MvfR is Involved in the Ability of U-50,488 and C4-HSL to Enhance PCN Production in PAO1.

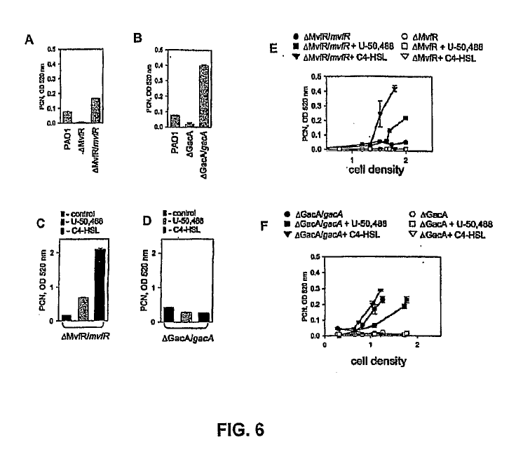

In order to define the possible role of MvfR and GacA in the U-50,488-mediated upregulation of PCN synthesis, we complemented ΔMvfR and ΔGacA with their respective genes on the multicopy plasmid pUCP24 (S. E. West, H. P. Schweizer, C. Dall, A. K. Sample and L. J. Runyen-Janecky, Gene 148, 81 (1994)). Both complemented mutants produced significantly higher amounts of PCN (FIG. 6A,B). The addition of C4-HSL and U-50,488 further increased the already elevated PCN production in ΔMvfR/mvfR (FIG. 6C). In contrast, PCN production in ΔGacA/gacA was decreased, albeit minimally, when exposed overnight to either 1 mM U-50,488 or 100 μM C4-HSL (FIG. 6D). Dynamic tracking of PCN production in the complemented mutant ΔMvfR/mvfR exposed to U-50,488 and C4-HSL demonstrated a shift in PCN production at lower cell densities (FIG. 6E), similar to that observed in the parental strain PAO1. The gacA complemented mutant, ΔGacA/gacA, itself produced PCN at lower cell densities than those observed with the parental strain PAO1. Exposure of ΔGacA/gacA to C4-HSL had no effect on the dynamics of PCN production whereas exposure to U-50,488 delayed PCN production. (FIG. 6F). These results indicate that MvfR is involved in the up-regulation of PCN production by exogenously applied U-50,488 and C4-HSL.

Intact Substrate-Binding and DNA-Binding Domains of MvfR are Required for U-50,488 to Enhance PCN Production in PAO1.

MvfR belongs to a family of prokaryotic LysR transcriptional regulators that possess a helix-turn-helix DNA-binding motif at the N terminus and a substrate binding domain at the C terminus. A NCBI Conserved Domain Search revealed similar domains in MvfR: a LysR DNA-binding domain located at 6-64 aa, and a LysR substrate binding domain located at 156-293 amino acids. Therefore PAO1 mutants were constructed producing N- and C-terminus-truncated MvfR to determine if specific domains could be identified that play a functional role in mediating the κ-opioid receptor agonist effect on PCN production. Results indicated that the mutant lacking amino acids 121-332, defective in the DNA-binding domain, did not produce any PCN, and did not respond to U-50,488 or C4-HSL. Mutants lacking either amino acids 1-299 or 1-293, truncated at their C termini without affecting the substrate binding domain, produced PCN and responded to U-50,488 and C4-HSL with enhanced PCN production. Further deletions, however, including amino acids Arg293, Leu292, and Phe284, did affect the substrate binding domain in mutants 1-292, 1-291, and 1-283. All three mutants failed to produce PCN and did not respond to U-50,488 and C4-HSL. These results confirm a key functional role for MvfR in mediating enhanced PCN production in P. aeruginosa in response to U-50,488 and C4-HSL.

The Effect of U-50,488 on PCN Production is Dependent on MvfR-Regulated Synthesis of Pseudomonas Quinolone Signal (PQS).

MvfR might play a critical role in PCN production via positive transcriptional regulation of the phnAB and PQS ABCDE operons that encode two 12 precursors of PQS, anthranilic acid (AA) and 4-hydroxy-2-heptylquinolone (HHQ) (E. Deziel, et al., Proc Natl Acad Sci USA 101, 1339 (2004)). Therefore the mutants ΔPhnA and ΔPqsA were examined for their ability to produce PCN in the presence of U-50,488. Neither mutant produced PCN. Exposure of each mutant to U-50,488 resulted in a slight increase in PCN production, although the increase was much less than that observed with the wild-type strain PAO1. These data suggested that MvfR-regulated PQS synthesis may be important for the ability of U-50,488 to enhance PCN production. Finally, reiterative experiments were performed with a P. aeruginosa mutant defective in the phzA1 gene, which is part of the operon that contains the core genes for PCN biosynthesis and that is directly preceded by the lux box (D. V. Mavrodi, et al., J Bacteriol 183, 6454 (2001)). ΔPhzA1 produced no PCN even when exposed to U-50,488.

To confirm that PQS plays a role in the pathway by which U-50,488 enhances PCN production, U-50,488 was applied to P. aeruginosa incubated with 2 mM methyl anthranilate (MA), a compound previously shown to inhibit PQS synthesis in P. aeruginosa (S. P. Diggle, et al., Mol Microbiol 50, 29 (2003), M. W. Calfee, J. P. Coleman and E. C. Pesci, Proc Natl Acad Sci USA 98, 11633 (2001)). Results demonstrated that MA inhibited the ability of U-50,488 to enhance PCN production in PAO1. These findings indicate that U-50,488 triggers PCN production in P. aeruginosa via a signal transduction pathway that includes the activation of transcriptional regulator MvfR and the synthesis of the MvfR-regulated molecule, PQS.

U-50,488 Stimulates other QS-Regulated Virulence Determinants in P. aeruginosa Including Biofilm Formation and PA-IL Production.

To determine if other QS-dependent phenotypes could be expressed in response to U-50,488, we measured biofilm production (T. R. De Kievit, R. Gillis, S. Marx, C. Brown and B. H. Iglewski, Appl Environ Microbiol 67, 1865 (2001)) and PA-IL lectin expression (K. Winzer, et al., J Bacteriol 182, 6401 (2000), M. Schuster, M. L. Urbanowski and E. P. Greenberg, Proc Natl Acad Sci USA 101, 15833 (2004)) in P. aeruginosa exposed to this opiate. U-50,488 enhanced biofilm formation in PAO1 in a concentration-dependent manner. PA-IL expression was dynamically tracked in response to U-50,488 using the green fluorescent PA-IL reporter strain P. aeruginosa 27853/PLL-EGFP (L. Wu, et al., Gastroenterology 126, 488 (2004)). Marked fluorescence was observed in this strain following 9 hours of growth in HDMEM media. Results were confirmed in strain PAO1 by immunoblotting using rabbit polyclonal antibody against PA-IL.

The Effect of U-50,488 on PCN Production in P. aeruginosa can be Inhibited by the Anti-Infective High Molecular Weight Polymer PEG 15-20.

A high molecular weight polymer, PEG 15-20, protects mice against lethal sepsis due to P. aeruginosa by interfering with the ability of both host elements (epithelial cell contact) and the QS signaling molecule C4-HSL to enhance P. aeruginosa virulence without affecting bacterial growth (L. Wu, et al., Gastroenterology 126, 488 (2004)). The capacity of PEG 15-20 to interfere with the U-50, 488 effect on P. aeruginosa was assessed by measuring PCN production in the media of P. aeruginosa PAO1 incubated in the presence of 5% PEG 15-20 and 0.5 mM U-50,488 or 0.2 mM C4-HSL. Results demonstrated that PEG 15-20 had a strong inhibitory effect on both U-50, 488- and C4-HSL-mediated up-regulation of PCN production.

TABLE 1
Bacterial strains

| P. aeruginosa strains | Relevant genotype |
|---|---|
| PA27853 | Wild type |
| PAO1 | Wild type |
| PAO-JP-1 | ΔLasI (lasl::Tc^{r}) |
| PAO-R1 | ΔLasR (lasR::Tc^{r}) |
| PDO100 | ΔRhlI (rhlI::Tn501) |
| PAO-MW1 | ΔRhlIΔLasI (rhlI::Tn501 lasI::tetA) |
| PAO44488 | ΔRhlR (rhlR:: ISphoA/hah) |
| PAO6281 | ΔGacA (gacA::Sp^{r}/Sm^{r}) |
| PAO6281/pUCP24/GacA | ΔGacA complemented with gacA |
| | on pUCP24 |
| PAO6281/pUCP24 | ΔGacA transformed with blank |
| | pUCP24 |
| PAO8902 | ΔMvfR (mvfR:: ISlacZ/hah) |
| PAO47418 | ΔMvfR (mvfR:: ISphoA/hah) |
| PAO35448 | ΔMvfR (myfR:: ISphoA/hah) |
| PAO51955 | ΔMvfR (mvfR:: ISphoA/hah) |
| PAO21170 | ΔMvfR (mvfR:: ISlacZ/hah) |
| PAO47853 | ΔMvfR (mvfR:: ISphoA/hah) |
| PAO47198 | ΔMvfR (mvfR:: ISphoA/hah) |
| PAO13375 | ΔMvfR (mvfR:: ISlacZ/hah) |
| PAO13375/pUCP24/MvfR | ΔMvfR complemented with mvfR |
| | on pUCP24 |
| PAO13375/pUCP24 | ΔMvfR transformed with blank |
| | pUCP24 |
| PAO53589 | ΔPqsA (pqsA:: ISphoA/hah) |
| PAO37309 | ΔPhzA (phzA:: ISphoA/hah) |
| PAO47305 | ΔPhzA1 (phzA1:: ISphoA/hah) |
| PAO3375/pUCP24/MvfR | ΔMvfR complemented with pUCP24 |
| 1-299 | harboring mvfR truncated with 33 aa |
| | at C terminus |
| PAO13375/pUCP24/MvfR | ΔMvfR complemented with pUCP24 |
| 1-293 | harboring mvfR truncated with 39 aa |
| | at C terminus |
| PAO13375/pUCP24/MvfR | ΔMvfR complemented with pUCP24 |
| 1-292 | harboring mvfR truncated with 40 aa |
| | at C terminus |
| PAO13375/pUCP24/MvfR | ΔMvfR complemented with pUCP24 |
| 1-291 | harboring mvfR truncated with 41 aa |
| | at C terminus |
| PAO13375/pUCP24/MvfR | ΔMvfR complemented with pUCP24 |
| 1-283 | harboring mvfR truncated with 49 aa |
| | at C terminus |
| PAO13375/pUCP24/MvfR | ΔMvfR complemented with pUCP24 |
| 121-332 | harboring mvfR truncated with 120 aa |
| | at N terminus |
| 27853/PLL-EGFP | Green fluorescent PA-IL reporter |
| | strain |

TABLE 2
Primers designed for complementation and truncation

| | | |
|---|---|---|
| Strain | Template | Primers |

| | | | |
|---|---|---|---|
| 13375/MvfR | PAO1 DNA | forward | 5′-AAGGAATAAGGGATGCCTATTCA-3′ |
| | | | SEQ ID NO: 3 |
| | | reversed | 5′-CTACTCTGGTGCGGCGCGCTGGC-3′ |
| | | | SEQ ID NO: 4 |
| PAO281/GacA | PAO1 DNA | forward | 5′-CGACGAGGTGCAGCGTGATTAAGGT-3′ |
| | | | SEQ ID NO: 5 |
| | | reversed | 5′-CTAGCTGGCGGCATCGACCATGC-3′ |
| | | | SEQ ID NO: 6 |
| 13375/1-299 | pUCP24/mvfR | MvfrXbaI | 5′-GCTCTAGAAAGGAATAAGGGATGCCTAT-3′ |
| | | | SEQ ID NO: 7 |
| | | C33HindIII | 5′-CCCAAGCTTCTAACGCTGGCGGCCGAGTTG 3′ |
| | | | SEQ ID NO: 8 |
| 13375/1-293 | pUCP24/mvfR | MvfrXbaI | 5′-GCTCTAGAAAGGAATAAGGGATGCCTAT-3′ |
| | | | SEQ ID NO: 7 |
| | | C39HindIII | 5′-CCCAAGCTTCTAGCGCAGGCGCTGGCGGGC-3′ |
| | | | SEQ ID NO: 9 |
| 13375/1-292 | pUCP24/mvfR | MvfrXbaI | 5′-GCTCTAGAAAGGAATAAGGGATGCCTAT-3′ |
| | | | SEQ ID NO: 7 |
| | | C40HindIII | 5′-CCCAAGCTTCTACAGGCGCTGGCGGGCGCT-3′ |
| | | | SEQ ID NO: 10 |
| 13375/1-291 | pUCP24/mvfR | MvfrXbaI | 5′-GCTCTAGAAAGGAATAAGGGATGCCTAT-3′ |
| | | | SEQ ID NO: 7 |
| | | C41HindIII | 5′-CCCAAGCTTCTAGCGCTGGCGGGCGCTTTC-3′ |
| | | | SEQ ID NO: 11 |
| 13375/121-232 | pUCP24/mvfR | N120XbaI | 5′-GCTCTAGAAAGGAATAAGGGATGGTCAGCCTGATACGC-3′ |
| | | | SEQ ID NO: 12 |
| | | MvfRHindIII | 5′-CCCAAGCTTCTACTCTGGTGCGGCGCGCTGGC-3′ |
| | | | SEQ ID NO: 13′ |

### Example 13A P. aeruginosa PAO1 Expresses Abundant PA-I and Alters MDCK Monolayer Permeability in a PA-1-Dependent Manner

In order to verify that the sequenced P. aeruginosa strain, PAO1, expressed PA-I, and to verify that strains altered the TEER of MDCK cells in a PA-1-dependent manner, both wild type and PA-I mutant strains deleted of the PA-I gene (leca) were assayed for PA-I protein expression and their abilities to decrease MDCK monolayer TEER. PA-I protein expression is highly abundant and responds to varying doses of C4-HSL, its cognate quorum sensing signaling molecule. In addition, in this strain, the ability of P. aeruginosa to decrease MDCK monolayer integrity (TEER) is highly dependent on the expression of PA-I. Also, it was determined that the PA-I induced permeability defect in MDCK cells was of sufficient magnitude to permit the apical to basolateral flux of exotoxin A across the monolayers, with a PA-1-induced TEER decrease of over 50% resulting in a five-fold increase in exotoxin A flux. Finally PA-I protein has been shown to be abundantly expressed in PAO1 when strains were exposed to the various opioid agonists. For PA-1 protein, the 6 agonist (BW373U86) induced a response equal to C4-HSL. The data establish that PA-I expression affects eukaryotic cell barrier function. Thus, it is expected that modulators of PA-I expression, as well as modulators of PA-I activity, will be useful in affecting the virulence phenotype of microbial pathogens and will be useful in affecting the eukaryotic (e.g., epithelial) cell barrier dysfunction associated with that phenotype.

### Example 14

### Host Cell-Derived Bacterial Signaling Components Enhance the Barrier-Dysregulating Properties of P. aeruginosa Against Epithelial Cells

In order to demonstrate that host stress BSCs could shift the barrier-dysregulating dynamics of P. aeruginosa against the epithelium, media and cell membrane fractions from Caco-2 cells exposed to hypoxia were added to the apical wells of MDCK cells apically inoculated with PA27853. TEER was measured over time. C4-HSL was also added to serve as a positive control for PA-I expression. Both media and cell membranes enhanced the barrier-dysregulating properties of P. aeruginosa (PA27853) against MDCK cells at four hours, at levels comparable to the level resulting from C4-HSL exposure. None of the host cell derived bacterial signaling compounds alone had any effect on MDCK TEER. The results demonstrate that the microbial pathogen (e.g., P. aeruginosa) is necessary to alter the barrier function of host cells.

### Example 15

### PA-I is Expressed In Vivo Within the Digestive Tube of Caenorhabditis elegans

The PA-1-GFP reporter plasmid was introduced into P. aeruginosa strain PA14, a strain highly lethal to C. elegans, by electroporation. Worms were then fed GFP-tagged PA14 and PA27853 and examined for fluorescent bacteria. Worms feeding on lawns of PA14 and PA27853 displayed fluorescent bacteria within the digestive tube, whereas no fluorescence was seen within the surrounding media, indicating that PA-I promoter activity is activated by local factors within the worm digestive tube. Finally the killing dynamics of strain PA-14, a highly lethal strain in this model, was compared to the dynamics associated with the completely sequenced PAO1 strain. The strain of E. coli (OP50) upon which worms normally feed, resulted in 100% survival, whereas, PA-14 displayed fast killing dynamics, as predicted. The PAO1 strain displayed slow killing with only a 50% mortality rate at 80 hours. Thus PAO1 exhibits killing dynamics that will allow assessments of whether host stress-derived BSCs shift the killing curve to that of a more virulent strain. It is expected that BSCs, whether soluble or membrane-bound, will shift the killing dynamics of relatively quiescent, or benign, microbes towards the dynamics exhibited by lethal microbial strains. Stated in the alternative, it is expected that a BSC will shift the phenotype of a microbe towards a virulent phenotype. Modulators of such activities are expected to be useful in preventing and treating disorders associated with the display of a virulence phenotype by any such microbe and in particular by P. aeruginosa. Such modulators are also expected to be used in methods for ameliorating a symptom of such a disorder.

### Example 16

### P. aeruginosa Genes Involved in BSC-Induced PA-I Lectin/Adhesin Gene Expression

The data demonstrate that i) morphine, the potent opioid agonists U-50488 and BW373U86, which target κ and δ receptors, respectively, and IFN-γ, induce a robust response in P. aeruginosa strains PA27853 and PAO1 to express PA-I; ii) PA-I expression is dependent on multiple elements of the virulence gene regulatory circuitry in P. aeruginosa, including the quorum sensing signaling system (QS) and RpoS. The data will show the genes that are required for opioids and IFN-γ to elicit a PA-1 response in P. aeruginosa and will facilitate a determination of whether these host stress-derived BSCs use common genes and membrane receptor proteins to activate PA-I expression.

A. Genes Required for P. aeruginosa PA-I Expression Responsive to Morphine, κ and δ Opioid Agonists, and IFN-γ

At least two techniques are contemplated for use in gene identification: 1) perform transcriptome analysis on P. aeruginosa strain PA01 exposed to morphine, κ and δ opioid receptor agonists, and IFN-γ, and 2) establish a functional role for candidate genes identified in the transcriptome analysis by screening the corresponding transposon mutants for their ability to up-regulate PA-I protein expression in response to opioids and IFN-γ.

### Transcriptome Analysis

Genes in strain PAO1 whose expression is increased in the presence of opioids and/or IFN-γ will constitute the initial focus. Transcriptome analyses is performed using Affymetrix GeneChip genome arrays in strain PAO1 to identify the genes that respond to the host cell elements such as morphine (non-selective opioid receptor agonist), U-50488 (K receptor agonist), BW373U86 (6 opioid receptor agonist), and IFN-γ. Time and dose variables for the following experiments are based on data for PA-I expression (mRNA) in strain PAO1.

Briefly, bacteria are grown in TSB overnight and diluted 1:100 in TSB containing either morphine (20 μM), κ agonist (80 μM), δ agonist (80 μM), or IFN-γ (10 μg/ml). Bacteria are then grown to an OD₆₀₀ of 0.5, 1.0, and 2.0, representing three stages of growth: exponential phase, late exponential phase, and stationary phase, respectively. These three time points will permit the capture of genes that are expressed early in the PA-I signaling pathway as well as during time points of high cell density. For transcriptome analysis, RNA is isolated from bacterial cells (treated and non-treated with morphine, K and 6 opioid receptor agonists, and IFN-γ) at the three designated points in the growth phase. cDNA synthesis, fragmentation, labeling, and hybridization, as well as P. aeruginosa GeneChip genome array processing, are performed as described herein or as known in the art. Each experiment is preferably performed in triplicate.

### Functional Analysis of Candidate Genes

Genes showing at least a 2.5-fold change in expression resulting from exposure to morphine, K and 6 opioid receptor agonists, and/or IFN-γ, are individually tested for their specific role in PA-I protein expression by screening mutant strains from a PAO1 transposon library (University of Washington Genome Center, see below) using dot blot analysis. Briefly, strains are grown in sequential runs using 384-well microtiter plates at 2 separate bacterial cell densities (OD₆₀₀ of 1.0 and 2.0) predetermined to respond to the inducing compound (opioids, IFN-γ). Dose-response curves are generated with varying doses of the PA-I inducing compounds at different bacterial cell densities in wild-type strains and in several mutant strains to determine the optimal conditions for screening. Experiments are performed separately for morphine, U-50488, BW373U86, and IFN-γ. Briefly, either morphine, U-50488, BW373U86, or IFN-γ are added to the wells containing mutant strains at the predetermined dose. All runs are performed with the wild-type strain as a control. The PA-I-inducing compound is added to the well for a predetermined time. Next, the supernatant is removed and the bacterial cell pellet is lysed by the addition of lysis solution directly into the well. The entire 384-well plate is then spun down (4000 g) and the supernatant transferred to an Immobilon P-PDF membrane using a 384 replicator. Membranes are then treated with anti-PA-I primary and secondary antibodies. Dot blot analysis allows for rapid identification of all of the mutant strains that do not up-regulate PA-I in the presence of host stress-derived bacterial signaling compounds, thereby identifying genes that are required for PA-I expression. All assays are preferably performed in triplicate (3 cell densities×5 groups (4 experimental+1 control)×triplicate (3) assays=45 gene arrays).

It is expected that many of the genes that have already been established to play a role in PA-I expression, including genes in the QS and RpoS regulon, will be identified. However, it is expected that new and unanticipated functions for known genes will also be identified. Further, if CyaB or GacS transcripts are increased in response to opioids or IFN-γ, and if Cya B and GacS transposon knockouts do not respond to either opioids or IFN-γ with an increase in PA-I, then the role of these established biosensors as two-component regulators of opioids or IFN-γ signaling to P. aeruginosa will be confirmed. Combining the results of the transcriptome analyses with the functional analyses of the transposon library will allow us to determine whether opioids and IFN-γ activate common membrane biosensors and common downstream genes involved in PA-I expression. It is possible that one or more of the non-peptide opioids diffuses directly into the bacterial cell cytoplasm where it initiates gene activation downstream of the two-component membrane biosensors. If this is the case, then all of the transposon knockout strains encoding membrane proteins are expected to respond with an increase in PA-I and microarray data will demonstrate that levels of transcripts encoding membrane proteins will be unaltered by either opioids or IFN-γ. However, it is possible that membrane biosensors are constitutively expressed and therefore gene expression will not change in response to opioids or IFN-γ. If this is the case, then the entire transposon library will be screened for PA-I expression in response to opioids or IFN-γ, approaches that are feasible given the high-throughput nature of the dot-blot technique. Of note here is that gene expressions can be triggered at different times during culturing and can respond to an exogenous compound(s) to varying degrees depending on the concentration of compound. The genomically sequenced strain PAO1 makes abundant PA-1 and the anti-PA-I lectin/adhesin antibodies are highly specific.

The data demonstrate that opioid receptor agonists and IFN-γ signal P. aeruginosa to express PA-I mRNA and protein. In addition, these PA-I signaling compounds induce P. aeruginosa to express a more virulent phenotype against the epithelium. The genes that control PA-I expression are dependent on two key global regulatory systems that activate hundreds of virulence genes in P. aeruginosa. The activation of these interconnected systems of virulence gene regulation are directly influenced by membrane biosensors that recognize elements of host cells and include, but are not limited to, CyaB and GacS, via a hierarchical cascade involving the transcriptional regulators Vfr and Gac A. Genes that are differentially expressed in response to opioids and IFN-γ will be identified using an unbiased transcriptome analysis approach. This approach was chosen instead of pursuing individual candidate genes involved in known pathways of PA-I expression because all previous studies have been performed only at high cell densities and in the absence of any host cell elements. Accordingly, previously described gene expression patterns may not be applicable in the physiologic models. The goal of this study is to identify and functionally validate the genes that are involved in PA-I expression in response to morphine, κ and δ opioid receptor agonists, and IFN-γ.

B. Identify the Receptors in P. aeruginosa that Bind Morphine and IFN-γ

The data show that a single solubilized membrane protein from P. aeruginosa can be isolated that avidly binds IFN-γ. In addition, morphine also binds to membrane protein fractions. Because antibody is available that specifically recognizes each of IFN-γ and morphine, initial studies are examining the effect of these two BSCs. Using the commercial antibodies, the membrane proteins that bind IFN-γ and/or morphine are identified, and optionally purified. This protein-based approach provides data which complements the experiments described above.

Two approaches available for use in identifying membrane proteins that bind IFN-γ and/or morphine are now described. First, membrane proteins of P. aeruginosa strain PAO1 are solubilized using mild detergents. The binding capacity of solubilized protein fractions for IFN-γ or morphine is then determined using simple ELISA binding assays. Protein fractions are then immunoprecipitated using the respective antibody and proteins are identified, e.g., by Maldi-MS.

Confirmation of the identity of a binding protein(s) is achieved by determining that a transposon knockout of the gene encoding the candidate protein(s) does not respond to IFN-γ or morphine with an increase in PA-I, using the techniques described herein. In order to confirm the function of candidate proteins showing fidelity in these two analyses, candidate proteins are re-expressed in the corresponding transposon knockout to verify that the PA-I response is re-established. Additionally, receptor antagonists may also be developed.

The data indicate that membrane receptors for morphine and IFN-γ can be identified by identifying proteins from solubilized membranes. A potential limitation using this technique is that morphine could diffuse directly into the bacterial cytoplasm and interact with a downstream target and not a membrane protein. This possibility is consistent with results demonstrating that morphine does not change the transcript profiles of any genes encoding membrane proteins, but the data for IFN-γ disclosed herein is inconsistent with this interpretation. In addition, morphine binding to a solubilized bacterial membrane protein was demonstrated using fluorescent imaging and analysis. Also, there is the possibility that transmembrane proteins or proteins that bind host stress-derived BSCs could be secreted into the culture medium and not be present within bacterial membranes. An example of such proteins are the bacterial iron binding proteins (enterochelin), which are released by bacteria into the culture medium and then re-enter the bacterial cells. Under such circumstances, the screening of cytosolic fractions and inner and outer membrane preparations are contemplated, along with iterative experiments probing for binding proteins with specific antibodies. Any discordance between the transposon mutant experiments and the proteins purified from bacterial membranes will be reconciled by analyzing IFN-γ-membrane protein or morphine-membrane protein interactions directly using surface plasmon resonance and mass spectrometry.

### Example 17

### The Impact of Host Signaling on Microbial Virulence States

The data demonstrate that PA-I knockout strains (lecA⁻) do not decrease the TEER of cultured epithelial cells. The lethality of strains of P. aeruginosa exposed to opioid agonists and IFN-γ can be defined in vivo using the well-characterized invertebrate, Caenorhabditis elegans, and the established model of gut-derived sepsis in mice.

A. The defect in Epithelial Barrier Function Induced by P. aeruginosa Exposed to Opioid Agonists and IFN-γ and the Role of PA-I in this Response

One issue is whether opioids or IFN-γ can activate P. aeruginosa to express a lethal phenotype against an epithelium, as judged by an increase in exotoxin A flux across epithelial cell monolayers, through the action of its PA-I lectin/adhesin.

To address that issue, MDCK cells are grown to confluence to maintain a stable TEER in transwells. Cells are apically inoculated with P. aeruginosa strain PAO1 (10⁷ cfu/ml) in the presence and absence of varying doses of morphine (about 20 μM), κ agonist (about 80 μM), δ agonist (about 80 μM), or IFN-γ (about 10 μg/ml). To optimize the effect of opioids and IFN-γ on the barrier-dysregulating effect of P. aeruginosa against epithelial cells, dose and time response curves are generated. TEER is measured using chopstick electrodes hourly for 8 hours. The apical to basolateral flux of exotoxin A using Alexa-594-labeled exotoxin A is determined in iterative experiments performed at each hourly time point in order to correlate the decrease in TEER to exotoxin A flux for each condition. In selected experiments in which a significant permeability defect to exotoxin A is established, the specific role of PA-I is defined by performing iterative experiments in the presence and absence of 0.3% GalNAc (N-acetylgalactoside) and 0.6% mellibiose, two oligosaccharides that specifically bind to PA-178. Irrelevant sugars (heparin/mannose) are used as negative controls. Iterative studies are also performed using the PA-I transposon knockout (lecA⁻) mutant to define the specific role of PA-I in strains exposed to opioids and IFN-γ. It is expected that PA-I will be expressed and localized to the microbial pathogen cell surface, where it will be situated in position to interact with host epithelial cells, thereby influencing, at a minimum, the cell barrier properties of the epithelial cells.

It is expected that opioids and IFN-γ will decrease the TEER of MDCK cells. Exotoxin A flux that is increased in cell monolayers with a low TEER will suggest that the opioids and IFN-γ alone can induce a lethal phenotype in P. aeruginosa. If the GalNAc, mellibiose inhibition studies, or the PA-I lectin/adhesin knockout strains, prevent P. aeruginosa from altering TEER and exotoxin A flux across the cell monolayers, then this will indicate that the observed response is PA-I-mediated. If the PA-I knockout mutant strains alter TEER and exotoxin A flux in response to opioids or IFN-γ, then this will indicate that PA-I alone may not be responsible for the virulence of P. aeruginosa against the intestinal epithelium. Data from these studies are directly compared and correlated to worm and mouse lethality studies (see below) to determine if these in vitro assays accurately predict a lethal phenotype in vivo, as expected.

### Example 18

### The Roles of Opioid Agonists and IFN-γ on Gut-Derived Sepsis due to P. aeruginosa as Revealed Using Caenorhabditis elegans and Surgically Stressed Mice

The data provide strong evidence that opioid agonists and IFN-γ enhance the virulence of P. aeruginosa in vitro through the action of PA-1. Yet the degree to which opioid agonists and IFN-γ influence the in vivo lethality of P. aeruginosa is unknown. Thus, the ability of opioids and IFN-γ to enhance the in vivo lethality of P. aeruginosa is assessed, e.g., in two complementary animal models.

Wild-type N2 Caenorhabditis elegans worms are grown to the L4 larval stage on normal growth medium (NGM) with E. coli OP50 as a nutrient source. Specialized agar plates are prepared onto which the PA-1-inducing compounds (vehicle (negative control)), opioids (morphine, κ and δ agonist), IFN-γ, and C4-HSL (positive control)) will be added and adsorbed into the agar as described for ethanol. The ability to embed bioactive compounds into the C. elegans growth agar is well described. Lawns of P. aeruginosa (wild type PAO1 and PA-I knockout PAO1 (lecA-)) are then grown on solid at agar plates by adding cultures of P. aeruginosa previously grown overnight in liquid media. Worms from the NGM medium are transferred onto the prepared culture dishes and killing dynamics assessed over time at temperature conditions of 25° C. Experiments are performed at different doses and re-dosing schedules to establish the optimum conditions under which a killing effect for each of the PA-1-inducing compounds can be demonstrated.

To test the ability of PA-I inducing compounds to enhance the lethality of P. aeruginosa in the established mouse model of gut-derived sepsis, mice are fasted for 24 hours and are subjected to general anesthesia, a 30% surgical hepatectomy, and cecal instillation of 10⁶ cfu/ml of wild-type PAO1 or PAO1 (lecA-) via direct puncture. Dose-response curves for P. aeruginosa in this model have been established and show that 10⁶ cfu/ml of P. aeruginosa induces a 50% mortality rate at 48 hours. In order to demonstrate that opioid agonists or IFN-γ enhance the lethality of P. aeruginosa in this model, varying doses of each are suspended in 1 ml of 0.9% NaCl and injected retrograde into the ileum in order to provide a constant supply of the PA-1-inducing compound for 24 hours. Normal saline alone is used for controls. This maneuver is known to be efficacious in delivering a continuous supply of an exogenous compound to the cecum in this model. Mice are fed water only for the next 24-48 hours and mortality recorded. Mice that appear moribund are sacrificed and the cecal mucosa, liver, and blood are cultured for P. aeruginosa growth on Pseudomonas isolation agar (PIA) in order to quantify bacterial adherence and dissemination patterns. The mice used in the study include two strains (wild-type+PA-I knockout) and, with 6 groups of 10 mice per group, a total of 120 mice is suitable.

Increased mortality in worms feeding on lawns of P. aeruginosa in the presence of opioids and/or IFN-γ demonstrates the ability of these compounds to induce a lethal phenotype in this organism against the intestinal epithelium. The demonstration of enhanced killing of worms in these experiments also serves to establish the feasibility and applicability of this model. As disclosed herein, in the absence of PA-1-inducing compounds, C. elegans displays a 50% mortality rate at 80 hours. In testing opioids and/or IFN-γ, or in screening for modulators of PA-I lectin/adhesin activity in general, it should be noted that, following 48 hours of growth and reproduction, worms can reproduce and progeny worms can be indistinguishable from the parent worms and overgrow the plates. If killing dynamics in response to PA-1-inducing compounds are such that observations extend past 48 hours, then use of a temperature sensitive mutant, e.g., C. elegans GLP4 (which does not reproduce at 25° C.), is preferred. Complementary experiments in mice will verify results obtained with worms.

The use of mouse studies to confirm results obtained with C. elegans preferably includes verification that luminally delivered PA-1-inducing compounds are efficacious in up-regulating PA-I as a general measure of enhanced virulence. To control for this possibility, experiments are performed to show that the PA-1-inducing compounds injected into the small bowel enhance PA-I expression in the mouse cecum. One approach involves the use of quantitative RT-PCR for PA-I and exotoxin A on freshly isolated RNA from cecal contents 24 hours following cecal instillation of P. aeruginosa. An alternative approach to delivering opioids and IFN-γ directly into the cecum is to engineer non-pathogenic E. coli strains that produce both morphine and IFN-γ. The feasibility of making recombinant morphine and IFN-γ in E. coli is well documented. Mice subjected to a surgical stress (e.g., hepatectomy) are then co-inoculated directly into the cecum with the LD₅₀ dose of P. aeruginosa (approximately 10⁶) and the morphine- and/or IFN-γ-producing E. coli strain. In this manner, P. aeruginosa would be directly exposed to a constant supply of the PA-1-inducing compound such as might naturally occur in vivo. Relevant here is the knowledge in the art that numerous microbial strains (E. coli Pseudomonas, Candida) naturally produce opioids, especially morphine. In addition, the “microbial soup” typical of a critically ill patient consists of highly pathogenic and resistant strains of bacteria that compete for nutrients in a highly adverse environment. Therefore, not only will the use of morphine- and/or IFN-γ-producing E. coli constitute a feasible alternative approach to obtaining in vivo mouse data, it may also recapitulate actual events in vivo. Finally, C. elegans normally feed on E. coli strains that do not induce mortality. The availability of morphine- and/or IFN-γ-producing E. coli strains could also be used in the C. elegans assays. Others have shown the feasibility of this approach is feasible in mice, as shown by delivering IL-10 into the intestinal mucosa of mice using direct intestinal instillation of bacteria that produce recombinant IL-10. The use of the C. elegans assay is expected to result in the rapid identification of therapeutics and prophylactics that modulate expression of a virulence phenotype by microbial pathogens in contact with, or proximity to, a mammal. The virulence phenotype is amenable to assessment using a variety of measures, many of them indirect, e.g., measurement of epithelial cell barrier function.

### Example 19

### Opioids and/or IFN-γ Release into the Intestinal Lumen Resulting from Host Stress

Endogenous morphine concentrations in the blood of humans and animals increase in direct response to the degree of surgical stress. The neural network of the mammalian intestine contains the most abundant concentration of opioid receptors in the body. Morphine has been recently shown to enhance the release of nitric oxide in the mammalian gastrointestinal tract via the μ3 opiate receptor subtype. In addition, it has been shown that the nematode, Ascaris suum, produces and liberates morphine in the gut. Similarly, IFN-γ has been shown to be released by the gut from intestinal intraepithelial lymphocytes in response to a variety of stressors, including bacterial challenge and ischemia/reperfusion injury (I/R).

To demonstrate that C. elegans produces or releases morphine, worms are grown permissively at 20° C. in massive cultures in liquid medium to 1×10⁶ worms using conventional culturing techniques. Stock cultures are treated with antibiotics 24 hours prior to the imposition of stress conditions. Worms are separated from any remaining bacteria by sedimentation and sucrose flotation as known in the art. Worms are then exposed to either heat stress (35° C. for 1 hour) followed by 2 hours of recovery, or hypoxic stress (0.3% O₂ for 45 minutes) followed by 1 hour of normoxic recovery, as described. Control worms are maintained at 20° C. and 21% O₂. Both the growth medium and the supernatant of homogenized C. elegans are preferably assayed for morphine by HPLC/GC/4S using conventional techniques. To determine whether morphine and IFN-γ are produced by, or released into, the mouse intestine following surgical stress, groups of mice (n=10/group) are subjected to a 30% hepatectomy or segmental mesenteric ischemia as described below. Surgical stress involving the hepatectomy model consists of performing a 30% surgical hepatectomy or sham laparotomy for controls and 24 hours later by harvesting the cecal tissue, the cecal luminal contents, and blood for morphine and IFN-γ assays. The ischemia reperfusion model (I/R) involves isolation of a 10 cm segment of distal ileum that is luminally cannulated and subjected to 10 minutes of ischemia (segmental artery clamp) followed by 10 minutes of reperfusion. Luminal perfusion with 2 ml of Ringers solution is performed to collect the luminal contents before and after I/R. Luminal contents, the homogenized intestinal segment, and blood are assayed for morphine by HPLC and GC/MS; IFN-γ is assayed by ELISA using a specific anti-IFN-γ antibody. A suitable number of mice for such assays is 30-50 mice.

Release of significant amounts of morphine and/or IFN-γ into the gut following surgical stress confirms that P. aeruginosa has been exposed to highly active compounds capable of activating or enhancing its virulence phenotype during host stress. In addition, a better understanding of the precise concentration of morphine and/or IFN-γ to which P. aeruginosa are exposed in vivo can be determined by these experiments. Whether morphine is released in high concentration in the lumen versus within the intestinal tissues is amenable to experimental determination. If luminal levels of morphine are elevated in hepatectomy versus controls, mice can be decontaminated with antibiotics (e.g., ciprofloxacin, metronidazole). Following such decontamination, the extent to which the luminal flora contribute to the opioid level can be determined using conventional techniques. It should be noted that, in addition to, e.g., morphine, other opioids and cytokines may be released from microbial pathogens such as P. aeruginosa that actively participate as host stress-derived BSCs. It is also possible that both opioids and IFN-γ are enzymatically degraded in the intestinal lumen. An alternative approach would be to use quantitative immuno-fluorescence of stained tissues to assess morphine and IFN-γ presence in tissues as antibodies specifically recognizing these compounds are readily available. Notwithstanding the preceding observations, these compounds have been measured by others from luminal contents without difficulty.

### Example 20

### Use of Knockout Mice to Confirm the Role of BSCs on PA-I Lectin/Adhesin Activity

IFN-γ is a key immune element that actively participates in both the local and systemic clearance of bacteria during acute infection. Animal models have shown that IFN-γ knockout mice have higher mortality rates following infectious challenge at local tissue sites (lung) compared to IFN-γ-sufficient mice in association with diminished ability to clear bacteria. Virtually all of the studies that have assessed the role of IFN-γ on P. aeruginosa infection in vivo have been performed in non-stressed mice where the infectious challenge has been instilled into the lung, and not in stressed mice, such as surgically stressed mice.

The lethality of intestinal P. aeruginosa is tested in IFN-γ knockout mice and wild-type controls (n=10 each group) in an established model of gut-derived sepsis. Mice fasted for 24 hours undergo 30% surgical hepatectomies followed by instillation of 10⁶ cfu/ml of wild type PAO1 into each cecum via direct puncture. Mice are then allowed water only for the remainder of the experiment and mortality is followed for 48 hours. Mice that appear moribund are sacrificed and the cecal mucosa, liver, and blood is quantitatively cultured on Pseudomonas isolation agar (PIA) to determine the rates of bacterial adherence and dissemination. To determine if PA-I expression in P. aeruginosa is attenuated in IFN-γ, a GFP PA-I reporter strain is injected directly into the cecum of mice subjected to a 30% hepatectomy and bacterial strains are recovered 24 hours later to determine fluorescence. The results of these experiments guide the performance of complementary studies using the segmental mesenteric ischemia model. Briefly, the lumena of 10 cm ileal segments subjected to sham ischemia (no clamp), 10 minutes of ischemia, and 10 minutes of reperfusion is perfused with Ringers solution and the timed aliquots of the perfusates is collected from both IFN-γ knockout mice and their wild-type cohorts. Use of the GFP-PA-1 reporter strains facilitates the determination of the extent to which each perfusate induces PA-I promoter activity. A suitable number of mice for such studies is 50 mice, divided into five groups with ten mice in each group.

The display of attenuated lethality by P. aeruginosa in IFN-γ knockout mice is consistent with IFN-γ playing a role as a host stress-derived bacterial signaling compound, or protein, during stress (e.g., surgical stress). IFN-γ may be only one of many signals necessary to orchestrate a fully lethal virulence repertoire for P. aeruginosa under the circumstances of catabolic stress, however. It is noted that IFN-γ knockout mice subjected to hepatectomy may develop an overcompensated and excessive inflammatory response to intestinal P. aeruginosa, resulting in increased mortality that is based more on immune response than enhanced microbial virulence. Tissue and blood culture results from these studies are used to determine whether mortality is due, in part, to such overcompensation. An alternative approach to distinguish between these possibilities is to perform studies in IFN-γ knockout mice and their matched wild-type cohorts (with and without surgical hepatectomy) to determine if there is a mortality difference when groups of mice are systemically inoculated (e.g., intraperitoneal, intravenous, lung instillation) with P. aeruginosa.

### Example 21

### Screens for Stress-Induced Bacterial Signaling Compounds

The data disclosed herein establishes that i) filtered luminal contents from the cecum of mice subjected to hepatectomy, or from the small bowel lumen of intestinal segments subjected to mesenteric arterial occlusion, induce a strong signal in P. aeruginosa to express PA-I; and ii) media and membrane preparations from hypoxic or heat-shocked Caco-2 cells induce PA-I expression.

A. Stress-Derived BSCs that are Present in the Media of Caco-2 Cells Exposed to Ischemia and Heat Shock Stress and that Induce PA-1 Expression in P. aeruginosa

Intestinal epithelial hypoxia is a common consequence of critical illness following surgical stress and is often an inadvertent consequence of its treatment. In addition, hyperthermia often develops during the acute stress response to injury and infection. Disclosed herein are data demonstrating that hypoxic or hyperthermic stress to cultured intestinal epithelial cells (Caco-2) causes the release of soluble PA-1-inducing compounds into the cell culture medium. This example discloses the isolation and identification of PA-1-inducing compounds that are released by Caco-2 cells exposed to hypoxia and hyperthermic stress.

Two sets of experiments are preferably performed. In the first set of experiments, Caco-2 cells grown to confluence in cell culture plates (150 cm²) are exposed to either normoxia (21% O₂) or hypoxia (0.3% O₂ for 2 hours followed by 1 hour of normoxic recovery). In the second set of experiments Caco-2 cells are exposed to normothermic (37° C.) or hyperthermic (immersed in water bath at 42° C. for 23 minutes followed by 3 hours recovery) conditions. Paired samples from each set of experiments are then processed to identify the specific host stress-derived bacterial signaling compound(s) using GFP-PA-1 reporter strains as a detection system. Media from Caco-2 cells is collected, filtered through a 0.22 μm filter (Millipore) and separated by molecular weight using centricones with a MW cutoff of 3, 10, 30, 50, 100 KDa (<3, 3-10, 10-30, 30-50, 50-100, >100 KDa). All fractions are preferably tested in 96 well plates to determine fractions that activate PA-I expression using PA-I GFP reporter strains. Two preferred approaches are contemplated for use in identifying the proteins that activate PA-I in the stress-conditioned media (hypoxia, hyperthermia). The first approach subjects bioactive fractions (i.e those that induce PA-I), and their molecular weight-matched control fractions (non-PA-1-inducing), to Maldi-Mass Spectrometry (MS) analysis. Spectra from the control media fractions are compared to the fractions of stress-conditioned media to determine the appearance of possible protein molecular ions present only in the samples that induce PA-I. This will allow us to subtract proteins that are present in both non-PA-1-inducing and PA-1-inducing fractions. In order to separate the molecular ion protein peaks that are present only in the PA-1-inducing fractions, bioactive fractions are loaded onto an HPLC equipped with a Vydac C4 column. Eluted samples are collected as fractions and individual fractions are tested for the ability to induce PA-I expression using the GPF-PA-I reporter strain. Proteins are then further separated, preferably by MW, hydrophobicity, and charge using stepwise well-controlled physico-chemical separation techniques in the HPLC system. Samples pre-fractionated in this manner should simplify the observed mass spectra and increase the likelihood of observing any putative protein(s) that induce PA-I expression. For any such proteins, identification using bottom-up proteomics techniques is performed.

An alternative to the use of molecular ion spectra, suitable in studies presenting highly complex spectra, is the classical approach for protein purification using conventional techniques such as ion exchange, hydrophobic, size exclusion, and/or affinity chromatography. Purification of host stress-derived BSCs is preferably assessed using the GFP-PA-I reporter strain.

For protein identification, protein-containing fractions are digested by using trypsin and digested fractions are analyzed with a LC/MSD XCT ion trap mass spectrometer system (Agilent Technologies, Santa Clara, Calif.). Data analysis for the data from the mass spectrometer is carried out using the SpectrumMill software platform (Agilent Technologies, Santa Clara, Calif.). Confirmation of the ability of identified proteins to induce PA-I expression is conveniently achieved in the PA-I:EGFP reporter strain by measuring fluorescence, and in P. aeruginosa strain PAO1 by immunoblot analysis.

Two protein fractions from Caco-2 cells that induce PA-I expression have been identified. Identification of specific active proteins (i.e., epithelial cell-derived PA-I signaling proteins) within the fraction(s) is achieved using any known technique, and preferably using a proteomics facility such as the University of Chicago proteomics facility. Many of these proteins may originate from the cell membranes themselves, since the most potent induction of PA-I expression occurs following contact with an epithelial cell membrane. In addition to protein identification, antibodies specifically recognizing such proteins are contemplated for such uses as cellular (e.g., Caco 2) localization studies. Although there are more classical approaches to protein identification, mass spectrometry is the most cost effective and rapid approach. For non-proteinaceous PA-I inducing compounds, lipid assays are contemplated that involve adjusting fraction pH to 3.5, followed by HPLC using, e.g., a Sep-Pak C₁₈ column. Eluted samples are trapped on a fraction collector, evaporated to dryness, and re-suspended in PBS for PA-I reporter assays. The structure of the active compound is preferably identified with IT/LC/MS/MS. For bacterial signaling compounds that are neither protein nor lipid, relevant fractions are resolved by IT/LC/MS/MS using a C₁₈ column and a quadrupole-time of flight mass spectrometer and NMR. Individual compounds are determined by their mass-fragmentation spectra, isolated, and tested for PA-I inducing activity using GFP reporter strains. Alternative approaches, such as 2D-SDS-PAGE electrophoresis for protein separation and TLC for non-protein separation, are also contemplated. Proteins separated by 2D-SDS-PAGE are typically transferred to a polyvinylidene difluoride transfer protein membrane for automated Edman degradation N-terminal sequence determination using an ABI 477A protein sequencer (Applied Biosystems). Protein identification is further facilitated by sequence comparison to database(s).

In addition to the foregoing screens for modulators, the invention contemplates any assay for a modulator of the expression of a virulence phenotype by a microbe in association with, or proximity to, a mammal such as a human. In particular, the invention comprehends a wide variety of assays for modulators of, e.g., eukaryotic cell barrier function, such as epithelial cell barrier function (e.g., epithelial cells of the intestine, lung, and the like). The invention further comprehends numerous assays for modulators of PA-I lectin/adhesin activity, whether due to a modulation of the specific activity of PA-I or a modulation of the expression of PA-I of constant specific activity, or both. In general, the invention contemplates any assay known in the art as useful for identifying compounds and/or compositions having at least one of the above-described characteristics.

### Example 22

### Miscellaneous Methods

### A. Screens for PA-I Modulators Using a PA-I Reporter Construct

Media from Caco-2 cells exposed to either hypoxia or heat shock stress induced PA-I expression in P. aeruginosa. Candidate PA-I inducer compounds that are released into the extracellular milieu following epithelial stress include ATP, lactate, cAMP, cytokines, and heat shock proteins.

The aforementioned candidate modulators, and other candidate modulators found in properly conditioned media, are identified using screening methods that constitute another aspect of the invention. Screens for such modulators are conveniently conducted in 96-well plates that contain the GFP-PA-I reporter strain PA27853/PLL-EGFP (see Example 24, below). The reporter strain is exposed to varying concentrations of candidate host stress BSCs including, but not limited to, heat shock proteins (HSP 25, 72, 90, 110), extracellular nucleosides and nucleotides (adenosine, ATP, cAMP) and cytokines (IL-1-18). Agents are added to the wells and dynamic assessment of bacterial fluorescence is carried out over 12 hours. Positive results are preferably verified by Western blot analysis of PA-I expression. For proteins that induce a PA-I response, the invention further comprehends assays to identify the receptors on P. aeruginosa to which such proteins bind. In one embodiment of this aspect of the invention, the identified protein inducer of PA-I activity is used as a probe to screen, e.g., a comprehensive library of P. aeruginosa by dot blot analysis. Confirmation of the screen results is available by assaying the protein-binding capacity of a lysate from a corresponding clone from a P. aeruginosa transposon library in which the relevant coding region has been disrupted by insertional inactivation.

Identified modulators are then subjected to additional in vitro and in vivo virulence assays to refine the understanding of the role in virulence expression played by such modulators.

### B. Caco-2 and MDCK Cell Culture, Measurement of TEER and Exotoxin A Flux.

Caco-2 cells and MDCK cells are well-differentiated epithelial cell lines that maintain a stable TEER when grown in confluent monolayer. Apical to basolateral exotoxin A flux across monolayers is assessed with Alexa 594-labeled exotoxin A using standard flux measurements.

### C. Bacterial Strains

P. aeruginosa strain PAO1 was obtained from the University of Washington Genome Center and is preferably used in the procedures disclosed herein, where appropriate.

D. Caenorhabditis elegans Assays.

Use of the nematode to assay for the lethality of P. aeruginosa is accomplished using standard protocols, as described herein.

### E. Antibodies.

Antibodies to PA-I are generated using conventional techniques. Preferably, such antibodies are purified by affinity chromatography. IFN-γ and morphine antibodies are commercially available.

### F. Dot Blot Assays for Membrane Binding.

ImmunoDot Blot assays for the detection of bacterial proteins in large matrix systems are known in the art. The technique has been validated as highly sensitive and accurate.

### G. Transcriptome Analysis of Bacterial Strain PAO1.

RNA is isolated from bacterial cultures exposed to opioids and/or IFN-γ as described herein at optical densities of 0.5, 1.0, 2.0. Between 1×10⁹ and 2×10⁹ cells are then mixed with RNA Protect Bacteria reagent (Qiagen) and treated as recommended by the manufacturer's mechanical disruption and lysis protocol. RNA is purified by using RNeasy mini columns (Qiagen), including the on-column DNase I digestion described by the manufacturer. In addition, the eluted RNA is preferably treated for 1 hour at 37° C. with DNase I (0.1 Upper μg of RNA). DNase I is then removed by using DNA-Free (Ambion) or by RNeasy column purification. RNA integrity is monitored by agarose gel electrophoresis of glyoxalated samples. Further sample preparation and processing of the P. aeruginosa GeneChip genome arrays are then done as described by the manufacturer (Affymetrix). For cDNA synthesis 12 μg of purified RNA is preferably combined with semirandom hexamer primers with an average G+C content of 75%, and Superscript II reverse transcriptase (Life Technologies). Control RNAs from yeast, Arabidopsis, and Bacillus subtilis genes are added to the reaction mixtures to monitor assay performance. Probes for these transcripts are tiled on the GeneChip arrays. RNA is removed from the PCR mixtures by alkaline hydrolysis. The cDNA synthesis products are purified and fragmented by brief incubation with DNase I, and the 3′ termini of the fragmentation products are labeled with biotin-ddUTP. Fragmented and labeled cDNA is hybridized to an array by overnight incubation at 50° C. Washing, staining, and scanning of microarrays is performed with an Affymetrix fluidic station.

### H. Expression Profiling.

The Affymetrix Microarray Software suite (MAS) (version 5.0) is a suitable software choice for determining transcript levels and whether there are differences in transcript levels when different samples are compared. Affymetrix scaling is used to normalize data from different arrays. A scale factor is derived from the mean signal of all of the probe sets on an array and a user-defined target signal. The signal from each individual probe set is multiplied by this scale factor. For any given array, between 18 and 28% of the mRNAs are considered absent by MAS, indicating that the corresponding genes are not expressed at levels above background levels. Furthermore, it is known in the art that the average changes in control transcript intensities are less than twofold for any comparison of array data. This indicates that the efficiency of cDNA synthesis and labeling is similar from sample to sample. For comparative analyses, the log₂ ratio for absolute transcript signals obtained from a given pair of arrays will be calculated by using MAS. A statistical algorithm of the software is also assigned a change call for each transcript pair, which indicates whether the level of a transcript is significantly increased, decreased, or not changed compared to the level for the baseline sample. The baseline samples are those derived from cultures of P. aeruginosa PAO-1 without any added opioids or IFN-γ. Graphical analyses of the signal log ratios from each experiment (any pair of arrays) is performed to display a normal distribution with a mean very close to zero (no change). Among the transcripts with significant increases or decreases compared to the baseline in one or more samples, those that showed at least a 2.5-fold change are subjected to further analysis. For cluster analyses and transcript pattern analyses, GeneSpring software (Silicon Genetics, Redwood City, Calif.) is contemplated as a suitable choice. The fold change values for each gene will be normalized independently by defining the half-maximal value for the gene as 1 and representing all other values as a ratio that includes that value. This scaling procedure will allow direct visual comparison of gene expression patterns within an experiment, as well as between experiments. GeneSpring is also contemplated for use in sorting genes according to the P. aeruginosa genome project.

I. Solubilization of Non-Denatured and Denatured Membrane Proteins Fractions from P. aeruginosa.

P. aeruginosa cells are washed with PBS and re-suspended in PBS containing a protein inhibitor cocktail. For preparation of membrane fractions, P. aeruginosa cells are disrupted by French pressure and centrifuged at 10000 g×30 minutes to eliminate debris. The supernatant is recentrifuged at 50000 g×60 minutes. The pellet is solubilized in 4% CHAPS at 37° C. for 3 hours. After being recentrifuged at 50000 g×60 minutes, the supernatant is spun through a 100K centricone and dialyzed against PBS. The binding capacity of the solubilized protein to γ-IFN is confirmed by ELISA binding assay.

### J. Statistical Analysis and Protein-Protein Interactions.

For statistical analysis, all data are preferably loaded into the SigmaStat platform software and appropriate tests applied. Protein-protein interaction studies are performed using conventional protocols, as would be known in the art.

### K. Maldi-MS Analysis.

Samples (0.5 μL) are mixed with an equal volume of a 5 mg/mL solution of α-cyanohydroxycinnamic acid in 30% acetonitrile in water with 0.1% TFA and are then manually spotted onto a 192 spot target plate (Applied Biosystems, Foster City, Calif.). The plate is inserted into a 4700 MALDI TOF/TOF (Applied Biosystems, Foster City, Calif.) operated in linear mode. Samples are desorbed by a 200 Hz YAG laser. The acquisition program is set to acquire a summed spectrum (200-1000) shots across the range 5000 to 100000 Thompsons.

### L. Digestion of a Protein Containing Fraction by Using Trypsin to Prepare for Protein Identification.

The protein extract sample is diluted in 50 mM ammonium carbonate buffer, pH 8.5, containing 0.1% Rapigest SF acid labile detergent (Waters Corp, Millford, Mass.). The sample is heated to 100° C. for 10 minutes to completely denature the proteins. Ten μL of 10 mM TCEP is added to reduce disulfide bonds and the sample is incubated for 10 minutes at 37° C. The pooled sample is digested with Lys-C (12.5 ng/μL) at a mass ratio of 1:100 for 3 hours at 37° C. and then digested with trypsin (12.5 ng/μL) at a mass ratio of 1:50 (trypsin:protein) for 3 hours at 37° C. Digestion is halted by adding PMSF to final concentration of 1 mM. After digestion, 10 μL of TFA is added to the solution and the sample is incubated for 45 minutes at 37° C. to destroy the acid labile Rapigest detergent.

### M. LC/MSD XCT Ion Trap Mass Spectrometry Analysis.

A digested protein sample is injected (10 μL) onto an HPLC (Agilent Technologies 1100) containing a C18 trapping column (Agilent Technologies, Santa Clara, Calif.) containing Zorbax 300SB-C18 (5×0.3 mm). The column valve is switched to its secondary position 5 minutes after injection and the trapped peptides are then eluted onto a 75 μm id Zorbax Stablebond (300 A pore) column and chromatographed using a binary solvent system consisting of A: 0.1% formic acid and 5% acetonitrile and B: 0.1% formic acid and 100% acetonitrile at a flow rate of 300 nL/minute. A gradient is run from 15% B to 55% B over 60 minutes on a reversed-phase column (75 μm id Zorbax Stablebond (300 A pore), and the eluting peptides are sprayed into a LC/MSD XCT ion trap mass spectrometer system (Agilent Technologies, Santa Clara, Calif.), equipped with an orthogonal nanospray ESI interface. The mass spectrometer is operated in positive ion mode with the trap set to data dependent auto-MS/MS acquisition mode. Source conditions are: Vcap −4500V, drying gas flow 8 L/minute, drying gas temperature 230° C. and CapEx 65V. The instrument is set to complete a mass scan from 400-2200 Thompsons in one second. Peaks eluting from the LC column that have ions above 100,000 arbitrary intensity units trigger the ion trap to isolate the ion and perform an MS/MS experiment scan after the MS full scan. The instrument's dynamic ion exclusion filter is set to allow the instrument to record up to 2 MS/MS spectra for each detected ion to maximize the acquisition of qualitative data from peptides (by preventing high abundance peptides from dominating the subsequent MS/MS experiments) and the excitation energy is set to “smart frag” mode to insure the generation of useful product ion spectra from all peptides detected. Data files that result are then transferred to a file server for subsequent data reduction.

N. The Mass Spectrometer Data Analysis with the SpectrumMill Software Platform.

SpectrumMill is derived from the MS-Tag software package and is contemplated as a suitable software platform. Raw data is extracted from the MS data files using the data extractor module and the data is then subjected to protein library search and de Novo spectral interpretation by the Sherenga module. SpectrumMill is designed to minimize spurious identifications obtained from the MS/MS spectra of peptides by careful filtering and grouping of related MS and MS/MS data during extraction from the raw data file. The library searching and de Novo interpretation identify the detected proteins form the individual peptides. The results for all proteins detected are collected and listed by protein name, detected peptide sequence(s), and search score. The reports are exported to an Excel spreadsheet file for inclusion in a result database. In addition, data extracted from the raw data files from the ion trap are preferably submitted to the Mascot (MatrixScience Inc, London, UK) search program and searched against both the NCBI non-redundant protein database and the SWISSPROT protein database. The identifications from these two systems are correlated to arrive at a final consensus list of identified proteins.

### O. Separation of Lipid Fractions on HPLC System.

Fractions are pH adjusted to 3.5, and run across a Sep-Pak C₁₈ column on a HPLC system (Millipore corp., Milford, Mass.). The columns are washed with ddH₂O, and compounds are eluted with increasingly polar mobile phases (hexane-methyl formate-methanol). Fractions are concentrated under a stream of nitrogen and reconstituted in either 1 ml PBS (for PA-I reporter assay) or 100 ul of methanol (for UV/HPLC). Active fractions from Sep-Pak are preferably further resolved by a C₁₈ reversed-phase HPLC column (150 mm×5 mm, Phenomenex, Torrance, Calif.) with acidified (0.1% acetic acid) MetOH:H₂O (60:40 vol/vol) at 1 ml/minute on a 1050 series HPLC using ChemStation™ software (Hewlett Packard, Palo Alto, Calif.).

### Example 23

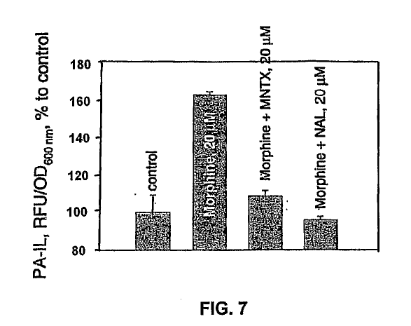

The separate effects of both tertiary and peripheral μ-opioid receptor antagonists on morphine-induced PA-I lectin/adhesin expression in Pseudomonas aeruginosa were investigated. The P. aeruginosa strain used for the study was the PA-I lectin/adhesin reported strain 27853/PLL-EGFP, described above. PA-I lectin/adhesin assays were performed as described herein except where specifically indicated. The reporter strain was incubated in wells of a 96-well plate, and fluorescence and cell density were measured using conventional techniques. Results presented in FIG. 7 represent fluorescence data normalized to cell densities after 20 hours of incubation. Bars represent median of twelve values ±stdv. Apparent from FIG. 7 is the effect of 20 μM morphine on PA-IL expression, as well as the separate inhibitory effects of each of 20 μM methylnaltrexone and 20 μM naloxone on the morphine-induced expression of PA-I lectin/adhesin.

As shown in FIG. 7, these opioid-induced increases in PA-I lectin/adhesin are significantly attenuated by either of the μ-opioid receptor antagonists, naloxone or methylnaltrexone. The effects on opiate-mediated virulence may be mediated through classical mu opioid receptors or in subtypes of opioid receptors or splice variants. Without wishing to be bound by theory, this effect may be mediated by MAPK/ERR phosphorylation similar to or related to VEGF. The data establish that both tertiary μ-opioid receptor antagonists, e.g., naloxone, and peripheral μ-opioid receptor antagonists, e.g., methylnaltrexone, are useful compounds, both prophylactically and therapeutically, in addressing the clinical effects of microbial pathogens on host organisms.

### Example 24

### Hypoxia-Induced PA-Lectin Adhesin Expression

The aim of the study described in this Example was to determine whether intestinal epithelial hypoxia, a common response to surgical stress, could activate PA-I expression. Because splancnic vasoconstriction and intestinal epithelial hypoxia are a common consequence of surgical injury, the aim of the experiments described herein was to determine the specific role of the intestinal epithelium in signaling to P. aeruginosa by examining the effect of epithelial cell hypoxia and reoxygenation on PA-I expression. A fusion construct was generated to express green fluorescent protein downstream of the PA-I gene, serving as a stable reporter strain for PA-I expression in P. aeruginosa, as described herein. Polarized Caco-2 monolayers were exposed to ambient hypoxia (0.1-0.3% O₂) for 1 hour, with or without a recovery period of nornoxia (21% O₂) for 2 hours, and then inoculated with P. aeruginosa containing the PA-I reporter construct. Hypoxic Caco-2 monolayers caused a significant increase in PA-I promoter activity relative to normoxic monolayers (165% at 1 h; P<0.001). Similar activation of PA-I was also induced by cell-free apical, but not basal, media from hypoxic Caco-2 monolayers. PA-I promoter activation was preferentially enhanced in bacterial cells that physically interacted with hypoxic epithelia. As shown below, the virulence circuitry of P. aeruginosa is activated by both soluble and contact-mediated elements of the intestinal epithelium during hypoxia and normoxic recovery.

### Human Epithelial Cells.

Caco-2_{BBe} cells expressing SGLT1 were maintained in DMEM with 25 mM glucose (high-glucose DMEM) with 10% fetal calf serum, 15 mM HEPES, pH 7.4, and 0.25 mg/ml geneticin, as previously described (Turner J R et al., Am J Physiol 273: C1378-1385, 1997). Caco-2 cells were plated on 0.33-cm² collagen-coated, 0.4-μm pore size polycarbonate membrane Transwell supports (Corning-Costar, Acton, Mass.) for 20 days, and media were replaced with identical media without geneticin at least 24 h before bacterial inoculation.

GFP Fusion Constructs of Wild-Type P. aeruginosa.

P. aeruginosa (ATCC-27853, American Type Culture Collection, Manassas, Va.) was transformed with the plasmid pUCP24/PLL-EGFP. This construct harbors a PA27853 chromosomal DNA fragment containing an upstream regulatory region of PA-I followed by the entire PA-I gene fused at the COOH terminal with an enhanced green fluorescent protein (EGFP) gene excised from the pBI-EGFP plasmid (Clontech, Palo Alto, Calif.). Expression of the PA-I lectin was detected by fluorescence microscopy and fluorimetry of this reporter strain as previously described (Wu L. et al., Ann Surg. 238, 754-764, 2003).

### Dynamic Fluorimetry.

Caco-2 cells were grown to confluence on collagen-coated 96-well fluorimetry plates (Becton Dickinson Labware, Bedford, Mass.) and maintained in a 37° C. incubator with 5% CO₂ and 21% O₂. The day before experiments, media were removed and replaced with 150 μl of antibiotic-free media. Three experimental conditions were created using a modification of the methods previously described by Xu et al. J Trauma 46:280-285, 1999). In control conditions, Caco-2 cells were maintained in a 5% CO2-21% O2 incubator for 2 h. Hypoxic conditions were achieved by placing Caco-2 cells in a humidified hypoxia chamber at 37° C. with 5% CO-95% N2 for 2 h. Measured O2 in the chambers varied between 0.1 and 0.3%. To simulate a reperfusion or reoxygenation state (normoxic recovery), after 2 h of Caco-2 cell hypoxia, hypoxic media were completely replaced with fresh, normoxic HDMEM media, and the cells were allowed to recover under normoxia (37° C., 5% CO₂-21% O₂) for 2 h before bacterial inoculation. The fluorescent reporter strain PA27853/PLL-EGFP was next added to the three groups of Caco-2 cells. Bacteria were cultured overnight in Luria-Bertani broth containing 20 μg/ml gentamicin at 37° C. under shaking conditions. After ˜12 h of growth, 50 μl of the bacterial suspension were added to the 96-well plates of Caco-2 cells. Care was taken to ensure that all bacterial samples were cultured for identical periods of time and that wells contained equal cell densities. Fluorescence was tracked immediately following bacterial inoculation and then hourly thereafter up to 3 h using a 96-well microplate fluorimeter (Synergy HT, Biotek, Winooski, Vt.). Plates were maintained in standard incubators at 37° C. with 5% CO₂-21% O₂ between all measurements. Fluorescence values were calculated as follows: % control=100×(RFUxₜ₌ₙ−RFUxₜ₌₀)/(RFUcₜ₌ₙ−RFUcₜ₌₀), where RFUx refers to the hypoxic or normoxic recovery groups and RFUc refers to the control at time n.

Exposure of Bacteria to Filtered Media from Caco-2 Cells and Potential PA-1-Inducing Candidate Molecules.

In this set of experiments, reiterative conditions of control, hypoxia, and normoxic recovery (i.e., reperfusion/reoxygenation) were created in 96-well plates containing confluent Caco-2 cells. Media from all wells were then collected and passed through a 0.22-μm filter and stored on ice. Ninety-six-well fluorimetry plates without Caco-2 cells (Costar 3631, Corning, Corning, N.Y.) were then prepared by adding a 20-μl bacterial suspension containing overnight growing cultures of PA27853/PLL-EGFP. Media from the three experimental groups were then added to the wells, and fluorescence was assessed over 5 h, with plates maintained at 37° C. with continuous orbital shaking (100 rpm) between measurements. To screen for potential PA-1-inducing compounds that might be present in the media of hypoxic Caco-2 cell media, purified adenosine, D-lactate, and L-lactate (Sigma-Aldrich, St. Louis, Mo.) were added to wells containing HDMEM across a range of physiologically relevant dosages, which were then tested as described above. Fluorescent microscopy.

To visually correlate results from the above experiments to the spatiotemporal effects of PA27853/PLLEGFP on hypoxic Caco-2 cells, cells were grown to confluence on Bioptechs dishes (Bioptechs, Butler, Pa.) and exposed to 2 h of hypoxia followed by inoculation with PA27853/PLL-EGFP. Experiments were performed on a 37° C. microscopy stage and visualized using an inverted fluorescence microscope (Axiovert 100, Carl Zeiss, Thornwood, N.Y.). Z-stacks were collected every 30 min for 3 h. Images were analyzed for bacterial distribution using ImageJ graphics analysis software (Version 1.31, National Institutes of Health, Bethesda, Md.).

Caco-2 Cell Barrier Function During Hypoxia and Normoxic Recovery in the Presence of P. aeruginosa or Purified PA-I.

Caco-2 monolayer transepithelial electrical resistance (TER), a measure of barrier function, was assessed using agar bridges and Ag—AgCl-calomel electrodes and a voltage clamp (University of Iowa Bioengineering, Iowa City, Iowa). TER was calculated using Ohm's law. Fluid resistance was subtracted from all values. Two microliters of overnight cultures of PA27853 normalized to cell density or 50 μg of purified PA-I (Sigma-Aldrich) were added to the apical chamber of the Caco-2 cell transwells following exposure to hypoxia and normoxic recovery as detailed above. Caco-2 cell TER was assessed every hour, and cells were maintained at 37° C. with 5% CO₂-21% O₂ throughout the experiment. To determine the effect of PA27853 on the barrier function of Caco-2 cells under conditions of sustained hypoxia, reiterative experiments were performed under continuous hypoxia (37° C., 5% CO₂-95% N₂), in which TER measurements were made every hour for 7 h within the hypoxic chamber using an EVOM resistance measurement apparatus (World Precision Instruments, Sarasota, Fla.).

### Northern Blot Analysis.

In selected experiments, PA-1 expression was confirmed using Northern blot analyses.

### Statistical Analysis.

Data were analyzed, and statistical significance was determined using Prism 4.0 (GraphPad Software, San Diego, Calif.). Statistical significance was defined as P<0.05 by Student's t-test or two-way ANOVA, as appropriate.

### Results

PA27853/PLL-EGFP P. aeruginosa Respond to the Environment of Caco-2 cell Hypoxia aid Normoxic Recovery with Enhanced Fluorescence.

To determine whether the green fluorescent protein (GFP) reporter strain PA27853/PLL-EGFP would display increased PA-I promoter activity when added to Caco-2 cells exposed to hypoxia (2 h at <0.3% O₂) and normoxic recovery (hypoxia followed by 2 h of recovery in normoxic conditions), reporter strains were added to the media of Caco-2 cells exposed to the two conditions. GFP reporter strains demonstrated significantly more PA-I promoter activity, as measured by fluorescence, within 1 h of incubation with Caco-2 cells exposed to either hypoxia or normoxic recovery. The media pH in all experimental conditions was measured at all time points and demonstrated no significant difference among control, hypoxia, and normoxic recovery groups because all media were buffered (data not shown). However, to show that the pH of media did not influence fluorescence in PA27853/PLL-EGFP, strains were incubated in media at pH 6.5, 7.4, and 7.7. The percent change in fluorescence was not different among groups (6.5=106±10, 7.4=100±12, 7.7=112±12; P=not significant). Similarly, to rule out an effect of hypercarbia or hypoxia alone on PA-I promoter activity in our reporter strains, strains were subjected to hypoxia (0.1% for 2 h) and hypercarbia (80% CO₂ for 2 h). No difference in fluorescence was observed between groups (data not shown). Taken together, these findings demonstrate that media from Caco-2 cells exposed to hypoxia with or without normoxic recovery activate PA-I promoter activity.

### Fluorescence Imaging of GFP Reporter Strains in the Caco-2 Cell Environment.

To determine whether epithelial cell contact contributes to the expression of GFP in our PA-I reporter strain, Caco-2 cells were imaged by fluorescent microscopy following exposure to hypoxia and apical inoculation with PA27853/PLL-EGFP. Fluorescence imaging demonstrated that PA27853/PLL-EGFP exposed to hypoxic Caco-2 monolayers appeared markedly more fluorescent than bacteria exposed to normoxic monolayers at the 120-min time point. Multiple images of the bacterial/Caco-2 cell coculture demonstrated that more bacteria were located near or within the plane of the cell monolayers exposed to hypoxia than in nonhypoxic cells. Quantitative analysis of multiple microscopy images revealed an average of 658±78 bacteria/high-powered field at the level of the surface of hypoxic epithelia, whereas no bacteria were seen in plane-matched controls (P<0.001).

PA27853/PLL-EGFP Reporter Strains Respond to a Paracrine Factor Present in Media from Caco-2 Cells Exposed to Hypoxia and Normoxic Recovery.

To determine whether soluble compounds released into the media in response to Caco-2 cell hypoxia are capable of activating PA-I expression independent of bacterial contact with the epithelium, we tested the ability of media from hypoxic Caco-2 cell cultures to enhance fluorescence in our reporter strain. PA27853/PLL-EGFP bacteria exposed to filtered media from Caco-2 cells exposed to hypoxia and normoxic recovery developed a significant enhancement of fluorescence that appeared greatest at the 5-h time point (FIG. 40; control: 3.7%±SD 3.9; hypoxia: 12.6%±SD 5.8; normoxic recovery: 13.1%±SD 3.9; P<0.001 by 2-way repeated measures ANOVA). Results were confirmed by Northern blot analysis. To determine whether this paracrine factor was isolated to the apical or basolateral compartments, we performed reiterative experiments in which isolated media from the basolateral and apical compartments of hypoxic monolayers, as well as mixtures of apical and basolateral media, were added to wells containing the GFP-PA-I reporter strain PA27853/PLL-EFGP. Only those bacteria exposed to hypoxic media from the apical chamber or hypoxic mixed media showed a statistically significant increase over controls (>125% change, normalized to initial value; P<0.05).

Adenosine Alone Induces PA-I Expression in P. aeruginosa.

To determine whether candidate compounds specifically released by hypoxic Caco-2 cells could induce the expression of PA-I, we tested the effect of D-lactate, L-lactate, and adenosine in our GFP-PA-I reporter strains. _{D-} and _{L-}lactate had no effect on PA-I promoter activity (data not shown); however, PLL/PA27853 responded with enhanced fluorescence to 10 mM adenosine, raising the possibility that adenosine released by hypoxic Caco-2 cells could be the putative mediator of the increased PA-I response observed in the above studies. However, the time required for upregulation of PA-I expression was longer than that observed in response to hypoxic cell media, suggesting that other factors may be involved in the signaling pathway.

### Caco-2 Cells Exposed to Hypoxia and Normoxic Recovery Resist the Barrier-Dysregulating Effect of Purified PA-1.

To determine whether conditions of hypoxia and normoxic recovery enhance or attenuate the barrier-dysregulating properties of PA27853 against Caco-2 cells, TER was measured in Caco-2 cells apically inoculated with either PA27853 or purified PA-I following exposure to hypoxia and normoxic recovery. Despite the ability of media from hypoxic and reoxygenated Caco-2 cells to increase the expression of PA-I in P. aeruginosa, the TER of Caco-2 cells exposed to these conditions were unchanged in response to a P. aeruginosa inoculated with purified PA-I exhibited an attenuated drop in TER compared with normoxic cells.

### Caco-2 Cells Exposed to Sustained Hypoxia Completely Resist the Barrier Dysregulating Effect of PA27853.

To determine whether Caco-2 cells exposed to sustained hypoxia could resist the barrier-dysregulating effect of PA27853, the TER of Caco-2 cells apically inoculated with PA27853 in an environment of sustained hypoxia was measured. Caco-2 cells maintained TER equal to hypoxic Caco-2 cells without bacteria and completely resisted the predicted decrease in TER at the 7-h time point. That Caco-2 cells partially resist the barrier-dysregulating effect of strains of PA27853 despite increased PA-1 expression could be explained by previous observations suggesting that epithelial cells normally respond to hypoxia with an enhancement of local mucosal defense proteins and barrier function.

### Soluble Factors Present in the Media of Hypoxic Caco-2 Cells Induce Increased Barrier Resistance in Normoxic Cells.

To determine whether the normoxic Caco-2 cells could be induced to increase their resistance to barrier dysregulation by P. aeruginosa through signals present in hypoxic cell media, we exchanged the apical and basolateral media of normoxic Caco-2 cells with filtered media from the apical and basolateral compartments of hypoxic Caco-2 cells and tested the barrier function of these cells when apically inoculated with P. aeruginosa. Normoxic Caco-2 cells exposed to media from hypoxicepithelia displayed a prolonged resistance to barrier dysregulation induced by P. aeruginosa, suggesting that normoxic epithelia may be activated to enhance their barrier function in the presence of soluble mediators produced during hypoxia.

Although P. aeruginosa is not considered to be an intestinal pathogen in the classic sense, it induces one of the most rapid and profound decreases in intestinal epithelial TER of any bacteria reported to date. We have previously reported, in both Caco-2 and T-84 cells, that P. aeruginosa (PA27853) can induce an 80% decrease in TER within 4 h following its apical inoculation. If defined by this criterion alone, P. aeruginosa is among the most pathogenic organisms to the intestinal epithelium yet described. The observation that as many as 5% of the normal population harbor this pathogen within their intestinal tracts, coupled with our animal studies demonstrating that control mice do not develop any symptoms of infection following the direct introduction of large quantities of P. aeruginosa into the cecum, suggest that this organism behaves like a classic opportunist, switching virulence genes on and off in response to selected environmental cues. Although it is well established that environmental cues such as pH, redox state, and nutrient composition can activate virulence gene expression in bacteria through a variety of membrane-bound biosensor kinases, there are no previous reports suggesting that bacterial signaling compounds are released by host cells following physiological or ischemic stress. From the standpoint of the evolutionary fitness of the microbe, however, it is logical that a pathogen might recognize the biochemistry of host cell stress, because possessing a system that recognizes host susceptibility would allow for a more accurate assessment of the costs versus benefits of host invasion. Yet, whereas it is well established that intestinal pathogens can communicate directly with the cells to which they adhere, that such a molecular dialogue might be bidirectional is poorly described.

To demonstrate that bacteria sense and respond directly to host cells, we used the PA-I lectin/adhesin of P. aeruginosa as a reporter gene. The PA-I lectin is under tight regulatory control of two key systems of virulence gene regulation in P. aeruginosa: the quorum-sensing signaling system and the alternative sigma factor RpoS. The quorum-sensing signaling system and RpoS are interconnected systems of virulence gene regulation in P. aeruginosa that control the expression of hundreds of virulence genes in this pathogen. Because PA-I expression is dependent on the function of both quorum sensing and RpoS, it serves as a relevant biological readout for generalized virulence gene activation in P. aeruginosa. The finding that soluble elements of intestinal epithelial cells and, in particular, adenosine can activate PA-I expression, suggests that specific host cell-derived compounds may be released that signal colonizing pathogens such as P. aeruginosa to a weak and susceptible host. That adenosine alone can activate PA-I expression is an important finding given that adenosine is released and can accumulate in the extracellular milieu of hypoxic tissues at high concentrations. During active intestinal inflammation, 5′-AMP derived from migrating polymorphonuclear leukocytes is converted to adenosine by the apical surface epithelium of the intestine. Strohmeier et al. (14) have demonstrated that under normal conditions, the human intestinal epithelial cell line T-84 can convert substantial amounts of 5′-AMP that accumulate to as much as 5 mM adenosine in the apical media within 30 min. Although in the present study, activation of PA-I promoter activity in P. aeruginosa required what appeared to be an unphysiological dose of adenosine, the precise concentration of adenosine to which P. aeruginosa might be exposed within the intestinal tract during prolonged hypoxia and reoxygenation is unknown. In addition, adenosine exposure required 6 h before PA-I promoter activity was observed, whereas with hypoxic media PA-I promoter activity was observed at 4 h. As a matter of speculation, an opportunistic organism like P. aeruginosa may require an inordinately potent and prolonged host-derived signal for it to invest the resources and energy required to mount a toxic offensive against the intestinal epithelium. Under such circumstances, P. aeruginosa might “sense” that the host on which its survival depends is subjected to an extreme degree of inflammation and vulnerability and hence represents a liability to its survival.

Given that PA-I expression was increased in response to Caco-2 cell hypoxia and normoxic recovery, we expected to see a more profound decrease in TER when P. aeruginosa was apically inoculated onto Caco-2 cells exposed to these conditions. That enhanced PA-I expression in P. aeruginosa did not decrease Caco-2 cell TER during hypoxia could be explained by the enhancing effect of hypoxia itself on Caco-2 cell barrier function. This possibility is supported by the finding that hypoxic media transferred to normoxic Caco-2 cells enhanced their resistance to P. aeruginosa. This notion is further supported by the finding that hypoxic Caco-2 cells resist the barrier-dysregulating property of purified PA-I, again suggesting that hypoxia enhanced epithelial barrier function to the barrier-dysregulating effects of the PA-1 protein of P. aeruginosa. These findings are also in agreement with the known enhancing effect of hypoxia on intestinal epithelial barrier function. Furuta and colleagues have demonstrated that exposure of Caco-2 cells to hypoxia increases the expression of both mucin and trefoil peptides, and they have also observed TER to be preserved or even increased in Caco-2 cells during hypoxia. This response makes physiological sense given that under such circumstances, the intestinal epithelial surface will be vulnerable to a potentially hostile flora. However, during reperfusion, which here we have termed normoxic recovery, Caco-2 cells eventually succumb to the potent barrier-dysregulating effect of P. aeruginosa. This is consistent with both clinical and animal studies where the greatest alteration in intestinal permeability and systemic proinflammatory activation occurs during the reperfusion phase following ischemic injury to the intestine.

In summary, herein we demonstrate that P. aeruginosa is capable of sensing and responding to local elements of host cell stress. Host-derived bacterial signaling compounds appear to be released by intestinal epithelial cells in response to hypoxia and normoxic recovery, which are often present during critical illness and its treatment. Further elucidation of the precise host compounds or signals that are sensed by colonizing nosocomial pathogens, such as P. aeruginosa, could lead to a better understanding of how infection continues to complicate the course of the most critically ill patients.

### Example 25

This study was designed to determine whether the intestinal tract of a stressed host is a unique environment in which the virulence of P. aeruginosa is enhanced in vivo. In order to further investigate this question, the inventors created a reporter strain of P. aeruginosa with GFP inserted downstream of the PA-I gene and the quorum sensing and RpoS promoters as described herein. To further understand how surgical stress and intestinal hypoxia might play a role in activating the virulence of P. aeruginosa, the inventors investigated whether HIF-1-α may play a central role in this response. It is well known that hypoxia results in the accumulation of HIF-1-α in intestinal epithelial cells. Given the increasingly important role of HIF-1-α activation in intestinal epithelial homeostasis, the investigators sought to determine if HIF-1-α activation mediates the release of soluble compounds that activate P. aeruginosa virulence as judged by expression of the PA-I lectin/adhesin.

To accomplish this an established Caco 2 cell line that has been stably transfected with HIF-1-a and its parental cell line were used. Briefly, both cell lines were grown to confluence. The media was collected and filtered through 0.22 u filters to remove any potential cellular components. Media was then added to microtiter wells containing a fixed bacterial cell population of the GFP/PA-I reporter strain described above. Fluorescence was dynamically tracked over time and was calculated according to the following formula:

$$\% of control = \frac{\frac{{RFU}_{HIF t = n}}{{OD}_{HIF t = n}} - \frac{{RFU}_{HIF t = 0}}{{OD}_{HIF t = 0}}}{\frac{{RFU}_{Control t = n}}{{OD}_{Control t = n}} - \frac{{RFU}_{Control t = 0}}{{OD}_{Control t = 0}}}$$

The results demonstrated that there is a time-dependent induction of PA-I expression observed in GFP/PA-I reporter strains exposed to HIF-1 α media compared to control (>400% and >600% change in PA-I expression in comparison to control at 7 and 8 hours, respectively, following inoculation). This finding were confirmed by Western blot analysis in reiterative experiments.

In order to identify the potential compounds that activate PA-I, the media from three groups of Caco-2 cells were examined, namely, control cells, Caco2 cells exposed to hypoxia, and Caco2 cells with forced expression of HIF-1 α. Media fractions were separated into 4 molecular weight fractions which were added to the microtiter plates containing the PA-I/GFP reporter strains and evaluated by dynamic fluorimetry.

Results from these experiments demonstrated that media fractions with MW of <3 kDa induced PA-I expression significantly (>800% and >700% increase in HIF-1-α and hypoxic media, respectively, at 0.7 hours following incubation).

Further studies were performed to show that HIF and hypoxic conditions have similar effects. Because of the MW of the potential inducing compound, the inventors examined the known genes that are expressed in response to HIF-1-α activation. Within this MW range we identified potential candidate compounds related to nucleotide metabolism. In particular, we were interested in adenosine since it has been shown to be released in high concentrations following intestinal epithelial hypoxia and HIF-1-α activation. Adenosine accumulates in the media of intestinal epithelial cells exposed to hypoxia and/or HIF-1a activation, through a mechanism that involves upregulation of 5′-nucleosidase (CD73) activity.

Therefore media fractions were examined by HPLC/MS/MS for adenosine by comparing 3 kDa centricon filtered media from control Caco-2 cells, hypoxic cells (0.1-0.3% O₂ for 2 hrs, and HIF-1-α overexpressing cells. Adenosine was greatly elevated in HIF-1-a activated and hypoxic cell media (>8000% increase).

When the effect of effect of adenosine on PA-I expression in the above-described reporter strain, it was seen that PA-I expression was increased in the presence adenosine that was both dose- and time-dependent (FIG. 8A). Results were confirmed by Western blot (inset in FIG. 8A). For completeness the effect of ATP, ADP, and AMP at similar concentrations was tested and revealed no evident inducing effect.

In order to determine if adenosine was the putative component within the media of HIF-1-α-activated Caco-2 cells that induces the expression of PA-I, adenosine deaminase was added to deplete the media of adenosine. Surprisingly, these experiments resulted in an even greater increase in PA-I expression, raising the possibility the metabolite of adenosine, namely inosine, plays a role in PA-I expression (FIG. 8). Adenosine deaminase is predicted to be present in P. aeruginosa based on its DNA sequence. In a related study inosine induced PA-I expression at a concentration 10-fold less than adenosine (FIG. 8C).

Reiterative experiments to directly compare the change in PA-I expression over time between inosine and adenosine demonstrate that not only is the effect of inosine greater, but it occurs at an earlier time point. Further studies showed that inosine induces PA-I expression at an earlier time point and at lower cell densities (OD) compared to adenosine.

In conclusion, the present example demonstrates that hypoxia or Forced expression of HIF-1-α in Caco-2 cells results in the extracellular release of soluble compounds that activate the virulence circuitry of P. aeruginosa. Further, the data presented herein show that adenosine and inosine may play an important role in this response.

### Example 26

This Example provides data establishing that a mu opioid receptor antagonist in the form of MNTX inhibits opiate-, thrombin- and LPS-induced endothelial cell barrier disruption by mu opioid receptor (mOP-R)-dependent, and -independent, mechanisms. The mOP-R-independent mechanisms of MNTX-induced endothelial cell barrier regulation include activation of receptor-like protein tyrosine phosphatase mu (RPTPμ) and inhibition of thrombin- and LPS-induced, Src-dependent, S1P₃ receptor transactivation (tyrosine phosphorylation). The results indicate that MNTX is useful as a cell barrier protective agent, such as an endothelial cell barrier protective agent. Although the data disclosed in this Example relate to pulmonary microvascular endothelial cells, the behavior of these cells exemplifies the behavior of any endothelial (or epithelial) cell towards opioid receptor agonists and antagonists. The data were generated using the following materials and methods.

### Materials and Methods

Cell Culture and Reagents—Human pulmonary microvascular endothelial cell were obtained from Cambrex (Walkersville, Md.) and cultured as previously described (2) in EBM-2 complete medium (Cambrex) at 37° C. in a humidified atmosphere of 5% CO₂, 95% air, with passages 6-10 used for experimentation. Unless otherwise specified, reagents were obtained from Sigma (St. Louis, Mo.). Morphine sulfate was purchased from Baxter (Deerfield, Ill.). Reagents for SDS-PAGE electrophoresis were purchased from Bio-Rad (Richmond, Calif.), Immobilon-P transfer membranes were from Millipore (Millipore Corp., Bedford, Mass.), and gold microelectrodes were from Applied Biophysics (Troy, N.Y.). Rabbit anti-mu opioid receptor antibody was purchased from Abcam (Cambridge, Mass.). Rabbit anti-SIPI receptor antibody was purchased from Affinity Bioreagents (Golden, Colo.). Mouse anti-S1P3 receptor antibody was purchased from Exalpha Biologicals (Watertown, Mass.). Mouse anti-RPTPμ antibody was purchased from Cell Signaling Technologies (Danvers, Mass.). Mouse anti-phospho-tyrosine antibody, mouse anti-pp 60src antibody and recombinant active Src were purchased from Upstate Biotechnologies (Lake Placid, N.Y.). PP2 was purchased from Calbiochem (San Diego, Calif.). Mouse anti-β-actin antibody, rabbit anti-phospho-tyrosine (418) Src antibody, naloxone, DAMGO, thrombin, LPS and ionomycin were purchased from Sigma (St. Louis, Mo.). Secondary horseradish peroxidase (HRP)-labeled antibodies were purchased from Amersham Biosciences (Piscataway, N.J.).

Immunoprecipitation and Immunoblotting—Cellular materials from treated or untreated HPMVEC were incubated with IP buffer (50 mM HEPES (pH 7.5), 150 mM NaCl, 20 mM MgCl₂, 1% Nonidet P-40 (NP-40), 0.4 mM Na₃VO₄, 40 mM NaF, 50 μM okadaic acid, 0.2 mM phenylmethylsulfonyl fluoride, 1:250 dilution of Calbiochem protease inhibitor mixture 3). The samples were then immunoprecipitated with anti-S1P₃ receptor IgG followed by SDS-PAGE in 4-15% polyacrylamide gels, transferred onto Immobilon™ membranes, and developed with specific primary and secondary antibodies. Visualization of immunoreactive bands was achieved using enhanced chemiluminescence (Amersham Biosciences).

Construction and Transfection of siRNA against mu opioid receptor, S1P₁, S1P₃, RPTPμ—The siRNA sequence(s) targeting human mOP-R, S1P₁, S1P₃, RPTPμ were generated using m-RNA sequences from Gen-Bank™ (gi:56549104, gi:87196352, gi:38788192, and gi:18860903, respectively). For each mRNA (or scramble), two targets were identified. Specifically, mOP-R target sequence 1 (5′-AACGCCAGCAATTGCACTGAT-3′; SEQ ID NO:14), mOP-R target sequence 2 (5′-AATGTCAGATGCTCAGCTCGG-3′; SEQ ID NO:15), S1P₁ target sequence 1 (5′-AAGCTACACAAAAAGCCTGGA-3′; SEQ ID NO:16), S1P₁ target sequence 2 (5′-AAAAAGCCTGGATCACTCATC-3′; SEQ ID NO:17), S1P₃ target sequence 1 (5′-AACAGGGACTCAGGGACCAGA-3′; SEQ ID NO:18), S1P₃ target sequence 2 (5′-AAATGAATGTTCCTGGGGCGC-3′; SEQ ID NO:19), RPTPμ target sequence 1 (5′-AATCTGAAGGTGATGACTTCA-3′; SEQ ID NO:20), RPTPμ target sequence 2 (5′-AACACCTTGACTAAACCGACT-3′; SEQ ID NO:21), scrambled sequence 1 (5′-AAGAGAAATCGAAACCGAAAA-3′; SEQ ID NO:22) and scramble sequence 2 (5′-AAGAACCCAATTAAGCGCAAG-3′; SEQ ID NO:23) were utilized. Sense and antisense oligonucleotides were provided by the Johns Hopkins University DNA Analysis Facility or were purchased from Integrated DNA Technologies (Coralville, Iowa). For construction of the siRNA, a transcription-based kit from Ambion was used (Silencer™ siRNA construction kit). Human lung endothelial cells were then transfected with siRNA using siPORTamine™ as the transfection reagent (Ambion, Tex.) according to the protocol provided by Ambion. Cells (about 40% confluent) were serum-starved for 1 hour followed by incubated with 3 μM (1.5 μM of each siRNA) of target siRNA (or scramble siRNA or no siRNA) for 6 hours in serum-free medium. The serum-containing medium was then added (1% serum final concentration) for 42 hours before biochemical experiments and/or functional assays were conducted.

Determination of tyrosine phosphorylation of the S1P₃ Receptor—Solubilized proteins in IP buffer were immunoprecipitated with mouse anti-S1P₃ receptor antibody followed by SDS-PAGE in 4-15% polyacrylamide gels and transfer onto Immobilon™ membranes (Millipore Corp., Bedford, Mass.). After blocking nonspecific sites with 5% bovine serum albumin, the blots were incubated with either mouse anti-S1P₃ antibody or mouse anti-phospho-tyrosine antibody followed by incubation with horseradish peroxidase (HRP)-labeled goat anti-rabbit or goat anti-mouse IgG. Visualization of immunoreactive bands was achieved using enhanced chemiluminescence (Amersham Biosciences).

Tyrosine Phosphatase Activity Assay—Treated or untreated HPAEC lysates and/or immunoprecipitated RPTPμ were analyzed for tyrosine phosphatase activity using the fluorometric Rediplate™ 96 EnzChek Tyrosine Phosphatase Assay Kit (Invitrogen (Molecular Probes), Eugene, Oreg.). Briefly, cellular materials were incubated in reaction buffer at 30° C. and then added to a 96-well plate coated with 6,8-difluoro-4-methylumbelliferyl phosphate (DiFMUP). Tyrosine phosphatase activity cleaves DiFMUP into DiFMU with excitation/emission maxima of 358/452 nm n.

In vitro SiP₃ Receptor Phosphorylation/Dephosphorylation—The S1P₃ receptor phosphorylation/dephosphorylation reaction was carried out in 50 μl of the reaction mixture containing 40 mM Tris-HCl (pH 7.5), 2 mM EDTA, 1 mM dithiothreitol, 7 mM MgCl₂, 0.1% CHAPS, 100 μM ATP, purified enzymes (i.e. 100 ng of recombinant active Src and/or immunoprecipitated RPTPμ obtained from MNTX-treated (1 hour) endothelial cells) and immunoprecipitated S1P₃ receptor obtained from human pulmonary endothelial cells that were serum-starved for one hour. After incubation for 30 minutes at 30° C., the reaction mixtures were boiled in SDS sample buffer and subjected to SDS-PAGE. Immunoblots were performed using mouse anti-phospho-tyrosine, mouse anti-pp 60src, mouse anti-RPTPμ or mouse anti-S1P₃ antibody followed by incubation with horseradish peroxidase (HRP)-labeled goat anti-mouse IgG. Visualization of immunoreactive bands was achieved using enhanced chemiluminescence (Amersham Biosciences).

Measurement of Endothelial Cell Electrical Resistance—Cell barrier properties were measured using a highly sensitive biophysical assay with an electrical cell-substrate impedance sensing system (Applied Biophysics Inc., Troy, N.Y.), as described previously in Garcia et al., Am. J. Physiol. 273:LI 72-L184 (1997); J. Appl. Physiol. 89:2333-2343 (2000); J. Clin. Invest. 108:689-701 (2000). The cells were cultured to confluence in polycarbonate wells containing evaporated small gold microelectrodes (10⁻⁴ cm²) and culture medium was used as electrolyte. The total electrical resistance was measured dynamically across the monolayer and was determined by the combined resistance between the basal surface of the cell and the electrode, reflective of focal adhesion, and the resistance between the cells. As cells adhered and spread out on the microelectrode, TER increased (maximal at confluence), whereas cell retraction, rounding, or loss of adhesion was reflected by a decrease in TER. The small gold electrode and the larger counter electrodes (1 cm²) were connected to a phase-sensitive Ion-in amplifier with a built-in differential preamplifier (Applied Biophysics). A I-V, 4000-Hz alternating current signal was supplied through a MQ resistor to approximate a constant-current source. Voltage and phase data were stored and computer processed using conventional techniques. Experiments were conducted only on cells that achieved >1000Q (10 microelectrodes per well) of steady-state resistance. Resistance was expressed by the in-phase voltage (proportional to the resistance), which was normalized to the initial voltage and expressed as a fraction of the normalized resistance value, as previously described (Garcia et al., (1997)). These measurements provided a sensitive biophysical assay that indicates the state of cell shape and focal adhesion reflective of changes in para-cellular permeability. TER values from each microelectrode were pooled at discrete time points and plotted versus time as the mean ±S.E.

Animal Preparation and Treatment—Male C57BL/6J mice (8-10 weeks, Jackson Laboratories, Bar Harbor, Me.) were anesthetized with intraperitoneal ketamine (150 mg/kg) and acetylpromazine (15 mg/kg) before exposure of the right internal jugular vein via neck incision. LPS (2.5 mg/kg) or water (control) were instilled intravenously through the internal jugular vein. Four hours later, mice received methylnaltrexone (MNTX, 10 mg/kg) or water control through the internal jugular vein. The animals were allowed to recover for 24 hours after LPS before bronchoalveolar lavage protein analysis and/or lung immunohistochemistry.

Mouse Lung Immunohistochemistry—To characterize the expression of proteins in mouse lung vascular endothelial cells, lungs from control (untreated) mice were formalin-fixed, 5 micron paraffin sections were obtained, hydrated and epitope retrieval was performed (DakoCytomation Target Retrieval Solution, pH=6.0, DakoCytomation, Carpinteria, Calif.). The sections were then histologically evaluated by either anti-mu opioid receptor, anti-RPTPμ or anti-S1P3 receptor antibody and secondary HRP-labeled polymer with DAB staining (Dako EnVision™+System, HRP (DAB) (DakoCytomation, Carpinteria, Calif.)), followed by hematoxylin QS counterstaining (Vector Laboratories, Burlingame, Calif.). Negative controls for immunohistochemical analysis were done by the same method as above but without primary antibody. Immunostained sections were photographed (100×) using a Leica Axioscope (Bannockburn, Ill.).

Determination of Bronchioalveolar Lavage Protein—Bronchioalveolar lavage (BAL) was performed by an intratracheal injection of 1 cc of Hank's balanced salt solution followed by gentle aspiration. The recovered fluid was processed for protein concentration (BCA Protein Assay Kit; Pierce Chemical Co., Rockford, Ill.).

Statistical Analysis—Student's t test was used to compare the means of data from two or more different experimental groups. Results are expressed as means ±S.E.

### Results

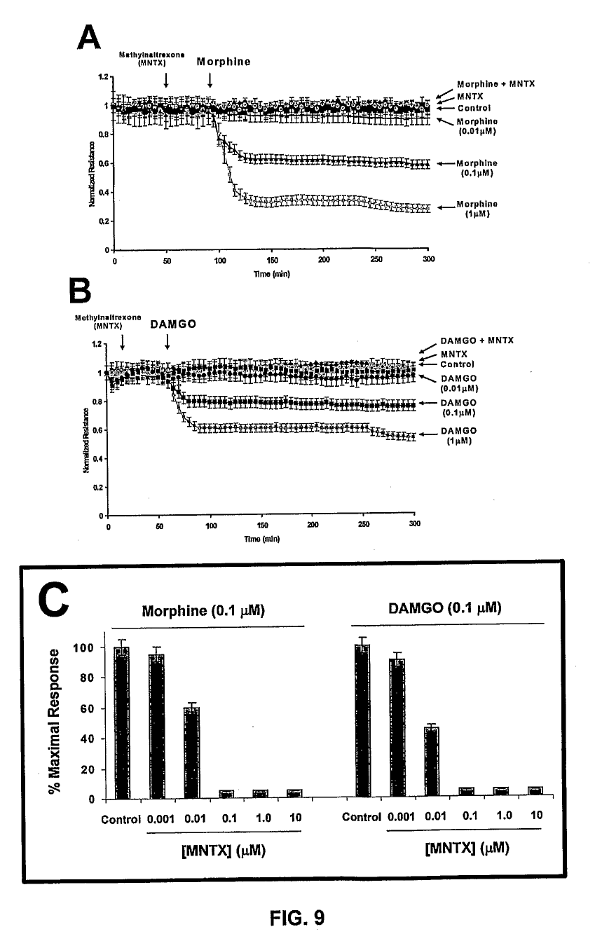

The role of methylnaltrexone (MNTX) in agonist-induced endothelial cell barrier disruption. Endothelial cell barrier disruption is a causative factor in a variety of pathologies, including atherosclerosis and acute lung injury. The effects of methylnaltrexone (MNTX), a charged peripheral mu opioid receptor (mOP-R) antagonist, on pulmonary microvascular endothelial cell integrity was examined using transendothelial resistance (TER). FIG. 9-A,B indicate that ligands for the mOP-R (i.e., morphine sulfate (MS) and DAMGO) induced endothelial cell barrier disruption in a dose-dependent manner. These barrier disruptive effects were blocked by pre-treatment with a physiologically relevant dose of MNTX (0.1 μM)). Decreasing the dose of MNTX below 0.1 μM attenuated its barrier protective effects while increasing the dose of MNTX beyond 0.1 μM did not significantly alter its actions (FIG. 9-C).

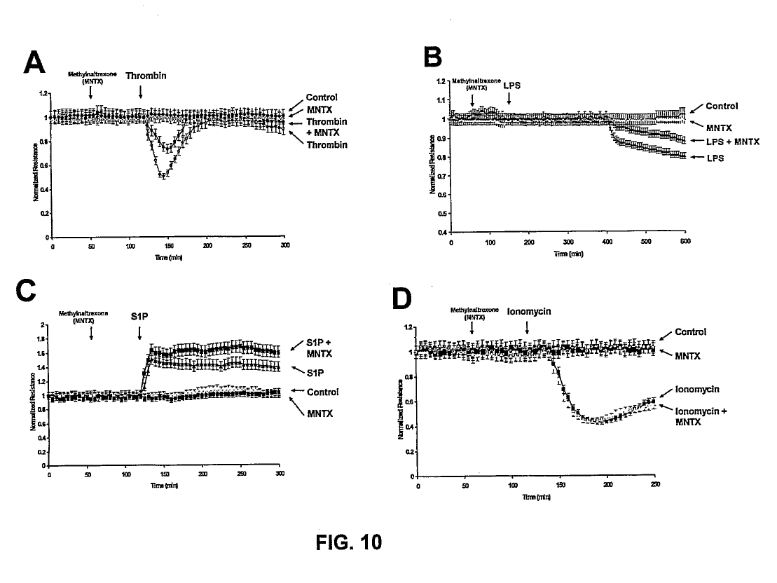

Next, the effects of MNTX on non-mOP-R-dependent agonist-induced endothelial cell barrier regulation were investigated. Thrombin induced a rapid transient decrease in endothelial cell barrier function (FIG. 10-A). Lipopolysaccharide (LPS) induced a delayed (about 4-hour) endothelial cell barrier-disruptive response (FIG. 10-B). MNTX (0.1 μM) attenuated endothelial cell barrier disruption from thrombin (FIG. 10-A) and LPS (FIG. 48-B) but not from the Ca²⁺ ionophore, ionomycin (FIG. 10-D). These results indicated selectivity in MNTX-mediated endothelial cell barrier protection. The protective effects of MNTX were not limited to barrier-disrupting agents, as MNTX increased the sustained endothelial cell barrier-enhancing effect of sphingosine-1-phosphate (S1P) (FIG. 10-C).

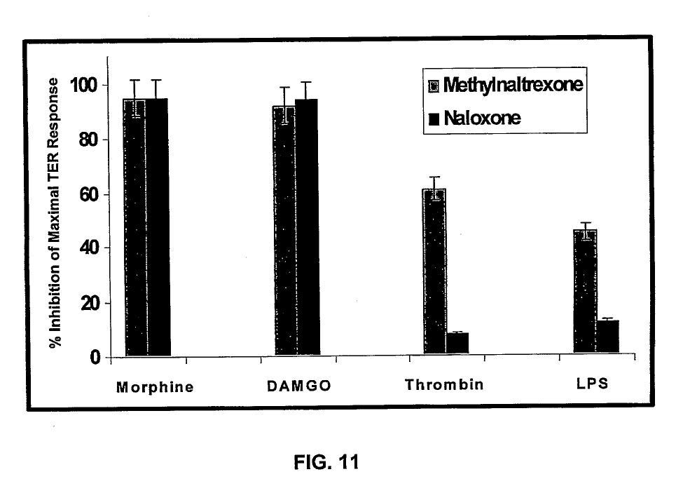

Methylnaltrexone is a charged molecule that cannot cross the blood-brain barrier (BBB). This property allows MNTX to selectively block peripheral mOP-R activity. The effects of another mOP-R antagonist, naloxone, which is uncharged and promotes both peripheral and CNS mOP-R inhibition, on agonist-induced endothelial cell barrier regulation were examined. Both MNTX and naloxone (0.1 μM) blocked MS- and DAMGO-induced endothelial cell barrier disruption. However, naloxone did not display the same endothelial cell barrier-protective effects as MNTX with thrombin and LPS challenge (FIG. 11).

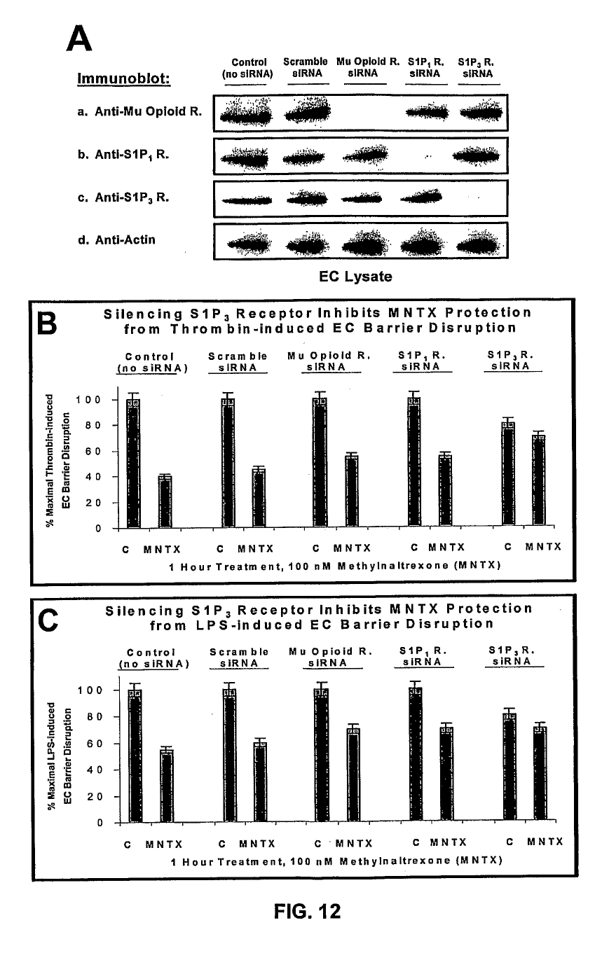

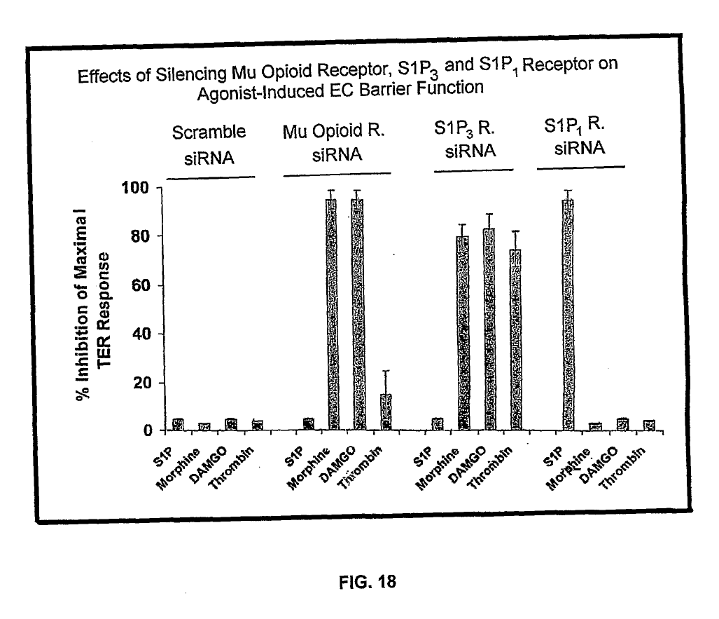

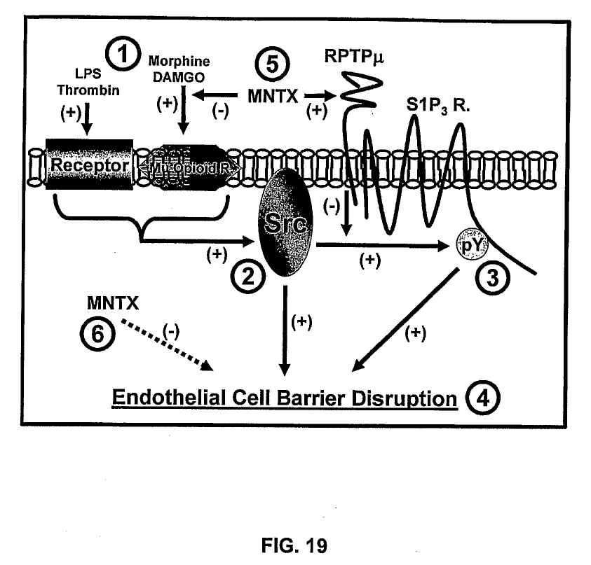

The role of S1P₃ receptor transactivation in agonist-induced endothelial cell barrier dysfunction. Considering the actions of MNTX on opiate and S1P-induced endothelial cell barrier regulation, the effects of silencing (siRNA) mOP-R or S1P receptor subtypes on MNTX-regulated endothelial cell integrity were investigated (FIGS. 12 and 18). Silencing mOP-R expression had little effect on MNTX protection from thrombin- and LPS-induced endothelial cell barrier disruption indicating potential mOP-R-independent effects of MNTX. Endothelial cells express both S1P₁ and S1P₃ receptors with S1P₁ receptor activating Rac1-mediated signaling, while S1P₃ receptor activates RhoA-mediated signaling. The silencing of S1P₁ receptor had previously been shown to completely eliminate the barrier-protective effects of SIP (1 μM). At higher concentrations (10 to 30 μM), S1P-induced barrier disruption is likely due to S1P₃ receptor activation. In contrast to S1P₁ receptor, silencing S1P₃ receptor inhibited thrombin- and LPS-induced, and MNTX protection from, endothelial cell barrier disruption (FIG. 12-B,C; FIG. 18).

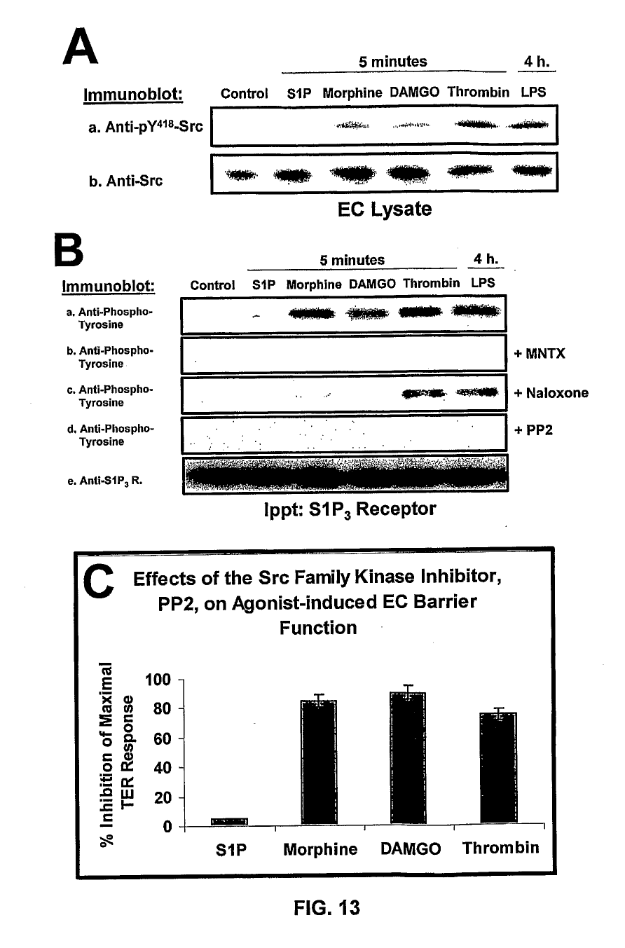

It is known that S1P₁ receptor transactivation is important in agonist-induced endothelial cell barrier enhancement. Considering the results in FIG. 12, it was expected that S1P₃ receptor transactivation would be an important regulatory mechanism in endothelial cell barrier disruption. FIG. 13 provides data indicating that barrier disrupting, but not barrier enhancing (i.e. S1P at 1 μM), agents promoted Src activation and Src family kinase-mediated S1P₃ receptor transactivation (tyrosine phosphorylation). Further, inhibition of Src family kinases by PP2 blocked agonist-induced barrier disruption but did not affect S1P-mediated endothelial cell barrier enhancement. Finally, pre-treatment with MNTX completely blocked agonist-induced S1P₃ receptor transactivation. In contrast, naloxone pre-treatment blocked the effects of morphine and DAMGO, but not thrombin or LPS, on S1P₃ receptor transactivation.

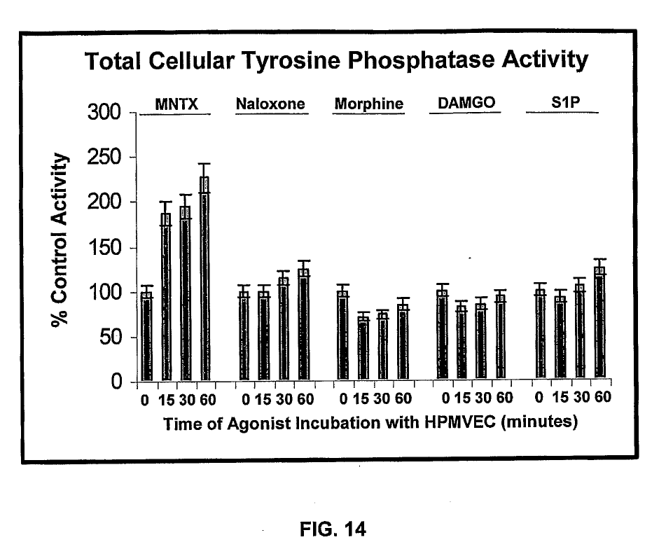

The role of receptor protein tyrosine phosphatase mu (RPTPμ) in MNTX-mediated protection from agonist-induced endothelial cell barrier disruption. The results in FIG. 13 indicated that MNTX blocked agonist-induced S1P₃ receptor transactivation (tyrosine phosphorylation). One mechanism of attenuating S1P₃ receptor tyrosine phosphorylation is through regulation of tyrosine phosphatase activity. The results indicated that MNTX (but not naloxone, morphine, DAMGO or SIP) increased total endothelial cell tyrosine phosphatase activity (FIG. 14).

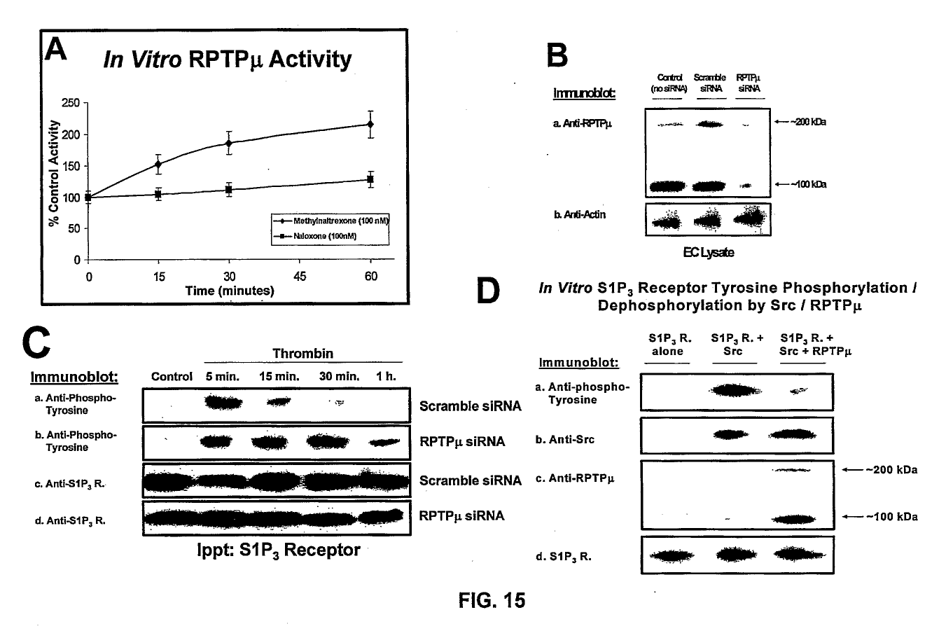

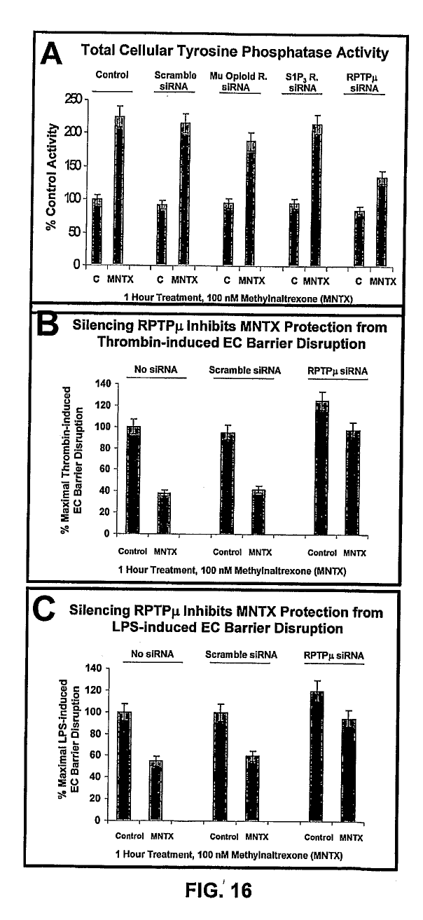

An important tyrosine phosphatase implicated in regulating human pulmonary endothelial cell-cell contacts is the receptor tyrosine phosphatase mu (RPTP). MNTX, but not naloxone, treatment of human pulmonary microvascular endothelial cells (HPMVEC) enhanced RPTPμ tyrosine phosphatase activity (FIG. 15-A). Further, silencing RPTPμ prolonged thrombin-induced S1P₃ receptor tyrosine phosphorylation (FIG. 15-B). In vitro analysis of isolated S1P₃ receptor indicated that MNTX-stimulated RPTPμ blocked Src tyrosine phosphorylation of the S1P3 receptor (FIG. 15-C). In addition, silencing RPTPμ (but not mOP-R or S1P₃ receptor) protein expression significantly attenuated the MNTX-mediated increase of total endothelial cell tyrosine phosphatase activity (FIG. 16-A). Finally, silencing RPTPμ inhibited the protective effects of MNTX of, and enhanced the thrombin- and LPS-induced effects on, endothelial cell barrier disruption (FIG. 16-B,C).

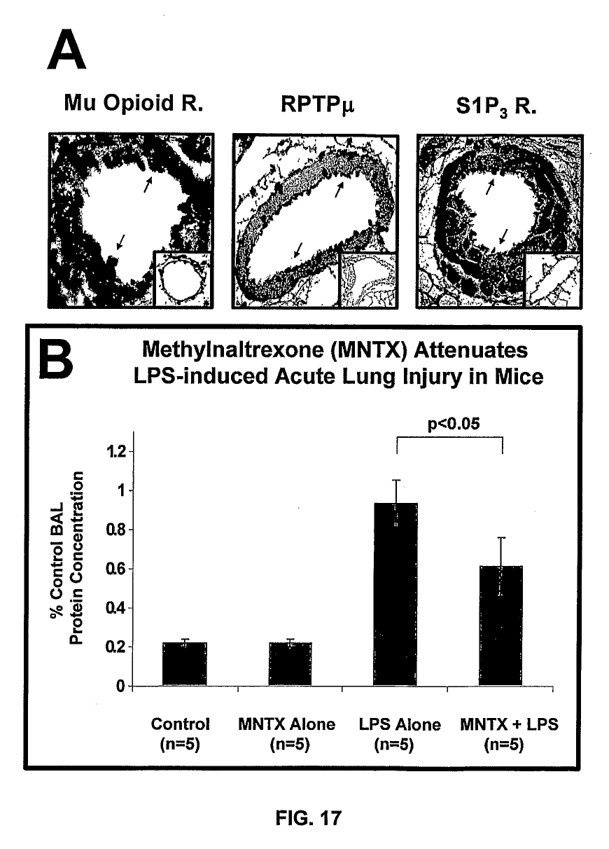

The role of MNTX in LPS-induced pulmonary vascular hyper-permeability in vivo. Similar to the results from human pulmonary microvascular endothelial cells, immunohistochemistry revealed that endothelial cells in mouse lung vasculature expressed mO P-R, PPTPμ and S1P₃ receptor (FIG. 17-A). Next, the effect of MNTX on LPS-induced endothelial cell barrier dysfunction in vivo was examined. Intravenous injection of LPS-induced endothelial cell-mediated vascular leakiness in mouse lung was measured by the protein concentration in bronchioalveolar lavage (BAL) fluid (FIG. 17-B). MNTX (10 mg/kg) alone did not affect pulmonary vascular permeability. However, intravenous injection of MNTX four hours after LPS delivery attenuated mouse pulmonary hyper-permeability (FIG. 17-B).

In this Example, data is presented that shows that methylnaltrexone (MNTX), a selective peripheral mu opioid receptor (mOP-R) antagonist, provided protection from agonist-induced endothelial cell barrier disruption through mOP-R-dependent, and -independent, mechanisms. The results indicate that S1P₃ receptor transactivation is an important regulator of agonist-induced endothelial cell barrier disruption. MNTX stimulated mOP-R-independent receptor tyrosine phosphate mu (RPTPμ) activity, which is important in inhibiting agonist-induced S1P₃ receptor transactivation (Src-mediated tyrosine phosphorylation). MNTX exhibited clinical utility for the treatment of diseases that involve cell barrier disruption, such as diseases associated with endothelial cell barrier dysfunction like atherosclerosis and acute lung injury.

The mu opioid receptor antagonist, naloxone, is fairly lipid-soluble and crosses the blood-brain barrier easily). Despite numerous attempts at regulating doses, mOP-R antagonists have proven unsuitable for patients receiving opiates for pain management because of analgesia reversal and breakthrough pain. MNTX is a quaternary derivative of the pure narcotic antagonist naltrexone. The addition of the methyl group to naltrexone at the amine in the ring forms the compound N-methylnaltrexone with greater polarity and lower lipid solubility. MNTX does not cross the blood-brain barrier and thus could play a therapeutic role in reversing the peripheral effects of opiates in palliative care, especially for patients taking high doses of opiates for analgesia. MNTX is expected to have a clinical role in the perioperative period, in the ICU (e.g., patients with burns), or with advanced medical illness. Because this population is most at risk for defects in cell barrier function, particularly pulmonary dysfunction, these work disclosed herein focused on MNTX rather than the tertiary opiate antagonists.

In previous studies of opiates and MNTX, the plasma concentrations of drugs appeared to be well within the range of the effects disclosed in the in vitro study. Peak plasma concentrations of intravenous or intramuscular morphine in normal therapeutic doses are 80 ng/ml. In one comprehensive review, analgesia in cancer patients was associated with steady-state concentrations of morphine in plasma ranging from 6 to 364 ng/ml. A meta-analysis of dose-adjusted peak plasma concentrations of morphine revealed a Cₘₐₓ of 1-10 nM/L per mg of morphine, although there were some differences between single- and multiple-dosing and populations. Taken as a whole, the plasma concentration of morphine and MNTX in patients after parenteral or oral administration is consistent with the levels that regulated endothelial cell barrier function in the in vitro model. Similarly, the concentrations of MNTX in the in vitro study were similar to those achieved in clinical trials of the drug. In methadone maintenance patients who received mean doses of 0.1 mg/kg MNTX intravenously, the mean plasma levels of MNTX were 162 ng/ml. After repeated IV doses of MNTX in volunteers, levels of MNTX in plasma were maintained well above the range in which we observed an effect on endothelial cell barrier function.

MNTX, but not naloxone, provided protection from both thrombin- and LPS-induced endothelial cell barrier disruption. Thrombin induced rapid, transient endothelial cell barrier disruption through activation of PAR (Protease-Activated Receptors), with consequent Ca²⁺, RhoA and Ras/MAP kinase signaling. In contrast, LPS induced a delayed endothelial cell barrier-disruptive response by activating a receptor complex of TLR4, CD14 and MD2, with consequent NF-KB activation and cytokine production. Considering the contrasting mechanisms of these agonists, MNTX is expected to provide cell barrier protection (including endothelial cell barrier protection) from a wide range of disrupting agents.

It is known that S1P₁ receptor transactivation (AKT-mediated threonine phosphorylation) is a key component in agonist-induced endothelial cell barrier enhancement. In this Example, these findings have been extended to show that transactivation (Src-mediated tyrosine phosphorylation) of the S1P₃ receptor played an important role in agonist-induced endothelial cell barrier disruption. S1P3 receptor signaling activated the small G-protein, RhoA, which is involved in actin cytoskeletal reorganization.

In agreement with these results, researchers have reported that inhibition of Src protected from endothelial cell barrier disruption. Src regulates endothelial cell contraction and vascular permeability. Inhibition of Src stabilized a VEGF receptor 2/cadherin complex and reduced edema after myocardial infarction.

RPTPμ was established herein as playing an important role in regulating endothelial cell barrier integrity. RPTPμ is highly expressed in the lung vasculature, where it is localized to endothelial cell-cell junctions. Consistent with the results disclosed herein, researchers have shown that silencing RPTPμ expression in HPMVEC inhibited barrier function. RPTPμ can associate with various cell surface receptors, including VE-cadherin, N-cadherin, c-Met and the VEGF receptor. These findings were extended to show that RPTPμ regulated S1P₃ receptor transactivation. RPTPμ further interacted with signaling molecules including IQGAP1, cdc42, RACK1, α-catenin, β-catenin and PKC6.

The in vivo model of pulmonary vascular permeability demonstrated that MNTX alone does not affect basal vascular integrity. However, MNTX attenuated LPS-induced vascular barrier disruption. These results are in agreement with the protective effects of MNTX on LPS-induced HPMVEC barrier disruption in vitro. Therefore, MTNX is expected to be a useful therapeutic treatment (including preventative and ameliorative treatments) for diseases involving cell barrier disruption or dysfunction, such as endothelial cell barrier dysfunction.

Having thus described at least one embodiment of each of several aspects of the invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method of preventing or treating a disorder characterized by a cell barrier dysfunction comprising administering to a subject free of an opioid-induced side effect an effective amount of a μ-opioid receptor antagonist.

2. The method according to claim 1 wherein said opioid-induced side effect is selected from the group consisting of opioid-induced constipation, irritable bowel syndrome, post-operative ileus, opioid-induced nausea, opioid-induced vomiting, pruritus, urinary retention, delayed gastrointestinal tract emptying, reduced gastrointestinal tract motility and opioid-induced suppression of the immune system.

3. The method according to claim 1 wherein the cell is an endothelial cell.

4. The method according to claim 1 wherein the cell is an epithelial cell.

5. The method according to claim 1 wherein the p-opioid receptor antagonist is a peripheral μ-opioid receptor antagonist.

6. The method according to claim 1 wherein the p-opioid receptor antagonist is selected from the group consisting of N-methylnaltrexone, alvimopan, ADL 08-0011, a piperidine-N-alkylcarboxylate, a quaternary morphinan, an opium alkaloid derivative and a quaternary benzomorphan compound.

7. The method according to claim 6 wherein the quaternary morphinan compound is selected from the group consisting of a quaternary salt of N-methylnaltrexone, N-methylnaloxone, N-methylnalorphine, N-diallynormorphine, N-allyllevallorphan and N-methylnalmefene.

8. The method according to claim 6 wherein the quaternary benzomorphan compound is selected from the group consisting of 2′-hydroxy-5,9-dimethyl-2,2-diallyl-6,7-benzomorphanium-bromide; 2′-hydroxy-5,9-dimethyl-2-n-propyl-6,7-benzomorphan; T-hydroxy-5,9-dimethyl-2-allyl-6,7-benzomorphan; 2′-hydroxy-5,9-dimethyl-2-n-propyl-2-allyl-6,7-enzomorphanium bromide; 2′-hydroxy-5,9-dimethyl-2-n-propyl-2-propargyl-6,7-benzomorphanium bromide; and 2′-acetoxy-5,9-dimethyl-2-n-propyl-2-allyl-6,7-benzomorphanium bromide.

9. The method according to claim 1 wherein said subject is a human patient.

10. The method according to claim 1 further comprising administration of a high molecular weight polyethylene glycol-like compound having an average molecular weight of at least 15 kilodaltons.

11. The method according to claim 1 wherein the p-opioid receptor antagonist is administered by a route selected from the group consisting of delivery by parenteral, oral, subcutaneous, transcutaneous, subcutaneous implantation, intramuscular, intravenous, intrathecal, intraocular, intravitreous, opthalmologic, intraspinal, topical, rectal, transdermal, sublingual, intramuscular, intracavity, aural, and nasal inhalation.

12. A method of reducing the risk of developing a disorder characterized by a cell barrier dysfunction comprising administering to a subject at risk of developing said disorder a prophylactically effective amount of a p-opioid receptor antagonist.

13. A method of preventing or reducing a symptom associated with a cell barrier disorder, comprising administering to a subject in need thereof a μ-opioid receptor antagonist, wherein the compound is administered in an amount effective to reduce at least one symptom of said disorder.

14. A method of preventing tumor cell metastasis comprising peri-operatively administering an effective amount of a p-opioid receptor antagonist to a patient having a tumor amenable to surgical intervention.

15. A method of inhibiting the expression of a bacterial PA-I lectin/adhesin comprising administering an effective amount of a μ-opioid receptor antagonist to a subject at risk of developing or suffering from bacterial pathogenesis.

16. A method of modulating the activity of a bacterial MvfR protein comprising administering an effective amount of a p-opioid receptor antagonist to a subject at risk of developing or suffering from bacterial pathogenesis.

17. A method of decreasing the permeability of, or preventing the increase in permeability of, an epithelium to a bacterial toxin comprising administering to a subject an amount of a μ-opioid receptor antagonist effective in reducing, or inhibiting an increase in, transepithelial cell electrical resistance.

18. The method according to claim 1, wherein the disorder is selected from the group consisting of gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.

19. A method of mitigating a cell barrier dysfunction free of p-opioid receptor-dependent effects, comprising administering to a subject free of an opioid-induced side effect an effective amount of a peripheral p-opioid receptor antagonist.

20. The method according to claim 19 wherein the peripheral μ-opioid receptor antagonist is N-methymaltrexone.

21. The method according to claim 19 wherein the cell barrier dysfunction is induced by an inducing agent selected from the group consisting of thrombin and bacterial lipopolysaccharide.

22. The method according to claim 19 wherein protein phosphatase is activated in said cell.

23. The method according to claim 22 wherein an S1P3 receptor phosphorylation is reduced.

24. The method according to claim 22 wherein a receptor protein tyrosine phosphatase μ is activated.

25. A method of mitigating a cell barrier dysfunction induced by transactivation of a S1P3 receptor, comprising administering to a subject free of an opioid-induced side effect an effective amount of a peripheral p-opioid receptor antagonist.

26. The method according to claim 25 wherein the peripheral p-opioid receptor antagonist is N-methylnaltrexone.

27. A method of using a p-opioid receptor antagonist in the preparation of a medicament for treating, ameliorating, or preventing a disorder selected from the group consisting of inflammation, atherosclerosis, acute lung injury, gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.

28. A method for preventing an infection or for lowering the risk of an infection comprising, administering to a patient in need of such treatment an effective amount of an opioid receptor antagonist.

29. The method according to claim 28, wherein the patient has or is expected to have a traumatic injury, an internal injury, a surgery, an acute lung injury, a burn, or high levels of stress.

30. The method according to claim 28, wherein the infection is from a bacterial opportunistic infectious agent.

31. The method according to claim 30, wherein the infectious agent is Clostridium dificile or Pseudomonas aeruginosa.

32. A method for preventing or treating inflammation comprising, administering to a patient in need of such treatment an effective amount of an opioid receptor antagonist.

33. The method according to claim 32, wherein inflammation patient has or is expected to have a traumatic injury, an internal injury, a surgery, an acute lung injury, a burn, or an infection.

34. A method for preventing or treating sepsis comprising, administering to a patient in need of such treatment an effective amount of an opioid receptor antagonist.

35. The method according to claim 34, wherein the administration is peri-operatively.

36. The method according to claim 12 wherein the disorder is selected from the group consisting of gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.

37. The method according to claim 13 wherein the disorder is selected from the group consisting of gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.

38. The method according to claim 15 wherein the disorder is selected from the group consisting of gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.

39. The method according to claim 16 wherein the disorder is selected from the group consisting of gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.

40. The method according to claim 17 wherein the disorder is selected from the group consisting of gut-derived sepsis, a burn injury, neonatal necrotizing enterocolitis, severe neutropenia, toxic colitis, inflammatory bowel disease, enteropathy, transplant rejection, pouchitis, pig-bel, Pseudomonas-mediated opthalmologic infection, Pseudomonas-mediated otologic infection and Pseudomonas-mediated cutaneous infection.
